(19) United States
(12) Patent Application Publication
(10) Pub. No.: US 2009/0214547 A1
(43) Pub. Date: Aug. 27, 2009

(54) BINDING MEMBER TOWARDS PNEUMOLYSIN

(76) Inventors: Anders Per Sorensen, Hillerod (DK); Thomas Lars Benfield, Copenhagen (DK); Jens Dilling Lundgren, Charlottenlund (DK); Thomas D. Kempe, Sunnyvale, CA (US)

Correspondence Address: BAKER BOTTS L.L.P., 30 ROCKEFELLER PLAZA, 44th Floor, NEW YORK, NY, 10112-4498, US

(21) Appl. No.: 11/660,877

(22) PCT Filed: Aug. 22, 2005
(86) PCT No.: PCT/DK2005/000536

§ 371 Date: Jul. 29, 2008

(30) Foreign Application Priority Data
Aug. 23, 2004 (DK) PA 2004 01271
Jul. 5, 2005 (DK) PA 2005 00989

(51) Int. Cl. A61K 39/395; C07K 16/12; C07H 21/00; C12N 15/63; C12N 5/10; G01N 33/569; C07K 14/195; A61K 39/02; A61P 31/04

(52) U.S. Cl. 424/139.1; 530/387.9; 530/387.3; 536/23.53; 435/320.1; 435/331; 435/7.32; 530/350; 424/190.1

(57) ABSTRACT

The present invention relates to an anti-haemolytic binding member comprising at least one binding domain capable of specifically binding Pneumolysin, in particular to a binding member having at least two biding domains, to the use of said binding members in diagnostic methods as well as for treatment. In a preferred embodiment the binding member is an antibody, such as a human antibody, or a fragment thereof, and it may also be a bispecific antibody.

The present invention relates to a binding member comprising at least one binding domain capable of specifically binding Pneumolysin, in particular to a binding member having at least two binding domains, to the use of said binding members in diagnostic methods as well as for treatment. Further described are Pneumolysin peptides and vaccine compositions comprising Pneumolysin peptides.

### BACKGROUND

Streptococcus pneumoniae is one of the leading causes of life-threatening bacterial infection. In developing countries it has been estimated that several million children under 5 years of age will die of S. pneumoniae each year (anonymous, 1985). In the industrialized world, the incidence of S. pneumoniae pneumonia is 5-10 per 100.000 persons and the case-fatality rate is 5-7%. S. pneumoniae meningitis occurs in 1-2 per 100.000 persons with a case-fatality of 30-40% (Lee et al., 1997). S. pneumoniae is one of the most frequent causes of bacteremia. S. pneumoniae is the most frequent organism isolated from children with otitis media. App. 75% of all children less than 6 years old will suffer from otitis media.

S. pneumoniae is a gram-positive bacterium that grows in pairs or short chains. The surface is composed of three layers: capsule, cell wall and plasma membrane. The capsule is the thickest layer and completely conceals the inner structures of growing S. pneumoniae. Polymers of repeating units of oligosaccharides (polysaccharides) dominate the capsule. Different serotypes contain ribitol, arabitinol or phosphorylcholine as part of their capsule, resulting in chemical structures that are serotype specific. The cell wall consists of peptidoglycan but also teichoic acid and lipoteichoic acid. The plasma membrane is a double phospholipid membrane that encompasses the cell and anchors various molecules to its surface (Alonso De Velasco, 1995).

At present 90 different types of S. pneumoniae are recognized based on the diversity of the S. pneumoniae capsule (Sorensen, 1995). The capsule is pivotal in the pathogenesis of S. pneumoniae infections. Antibodies raised against one capsular type offers protection from infection with this type but not against infection with other capsular types. The current 23-valent polysaccharide vaccine offers protection from more than 60-85% of the most frequent serotypes.

Pneumolysin is a major virulence factor of some gram-positive bacteria and is a member of a family of cholesterol-binding toxins (de los Toyos et al., 1996). It is a soluble protein that disrupts cholesterol-containing membranes of cells by forming ring-shaped oligomers (porins) (Bonev et al., 2001). Further, Pneumolysin activates the complement system in a non-specific manner through interaction with Fc and complement proteins. The toxicity of Pneumolysin can be attenuated by site-directed mutagenesis (Trp-433 to Phe substitution) of the Pneumolysin gene, resulting in the expression of pneumolysoid (PdB) (Alexander et al., 1994).

Pneumolysin appears conserved among tested S. pneumoniae strains (Paton et al., 1983). The deduced amino acid sequence based on the Pneumolysin gene from different strains of S. pneumoniae is >99% identical (Mitchell et al., 1990).

IgA to Pneumolysin is detectable in saliva from children (242 of 261) and adults (17 of 17) (Simell et al., 2001). Anti-Pneumolysin IgG was detectable by EIA in most children less than two years (803 of 1108) and all adults (325/325) (Rapola et al., 2000). Seroconversion was correlated to carrier status, i.e. children who had been infected with S. pneumoniae cultured from nasopharyngeal or middle ear specimens were more likely to be anti-Pneumolysin IgG positive. In a different study using an ELISA method, IgG was detected in 7 of 40 healthy adults, 17 of 32 patients with chronic obstructive pulmonary disease, and 13 of 31 patients with pneumococcal pneumonia (Musher et al., 2001). Interestingly, significantly fewer patients with pneumonia and bacteremia had detectable IgG compared to patients with pneumonia but without bacteremia (4/16 vs. 9/15). This suggests that anti-Pneumolysin antibodies may prevent pneumonia from progressing to bacteremia.

### SUMMARY

The present invention relates to an anti-haemolytic binding member comprising at least one binding domain capable of specifically binding Pneumolysin, wherein the binding member is suitable for use in a pharmaceutical composition for preventing and treating diseases and disorders related to Streptococcus, in particular Streptococcus pneumoniae.

Accordingly, in one embodiment the invention relates to an isolated binding member comprising at least one binding domain capable of specifically binding Pneumolysin, said binding domain having a dissociation constant K_{d} for Pneumolysin which is less than 1×10⁻⁶. Preferably the binding member comprising the binding domain has the dissociation constant K_{d} defined above.

Due to the high binding strength the binding member is suitable for use in a pharmaceutical composition. Further more binding members with anti-haemolytic activity are particular useful.

In another aspect the invention relates to an isolated binding member comprising at least a first binding domain and a second binding domain, said first binding domain being capable of specifically binding Pneumolysin.

The binding member according to the invention is preferably an antibody or a fragment of an antibody. The antibody may be produced by any suitable method known to the person skilled in the art, however it is preferred that at least a part of the binding member is produced through a recombinant method. Accordingly, the present invention relates in one aspect to an isolated nucleic acid molecule encoding at least a part of the binding member as defined above, as well as to a vector comprising the nucleic acid molecule defined above, and a host cell comprising the nucleic acid molecule defined above.

The invention further relates to a cell line engineered to express at least a part of the binding member as defined above, and more preferably engineered to express the whole binding member as defined above.

In a further aspect the invention relates to a method of detecting or diagnosing a disease or disorder associated with Pneumococcus in an individual comprising
providing a biological sample from said individual,
adding at least one binding member as defined above to said biological sample
detecting binding members bound to said biological sample, thereby detecting or diagnosing the disease or disorder.

Also, in the method the invention further relates to a kit comprising at least one binding member as defined above, wherein said binding member is labelled, for use in a diagnostic method.

In yet another aspect the invention relates to a pharmaceutical composition comprising at least one binding member as defined above.

Furthermore, the invention relates to the use of a binding member as defined above for the production of a pharmaceutical composition for the treatment or prophylaxis of disorders or diseases associated with Streptococcus pneumoniae, such as pneumonia, meningitis and/or sepsis.

In yet a further aspect the invention relates to a method for treating or preventing an individual suffering from disorders or diseases associated with Streptococcus pneumoniae, such as pneumonia, meningitis and/or sepsis by administering an effective amount of a binding member as defined above.

Further aspects relates to a Pnemolysin peptide recognized by an anti-haemolytic binding member and a vaccine composition comprising such peptide.

### DRAWINGS

FIG. 1. Schematic drawing of a Fab fragment.

FIG. 2. Pneumolysin amino acid sequence having SEQ ID NO 11.

FIG. 3. Anti-Pneumolysin light chain and heavy chain variable segment.

FIG. 4. Survival diagram for mice inoculated with Pneumococcus and antibody.

FIG. 5. Antihaemolytic activity of Pneumolysin antibodies

FIG. 6 Peptides for epitope mapping.

FIG. 7 Graphic illustration of determination of Pneumolysin antibody epitopes.

FIG. 8 Isolation of 26-5F12 clones

FIG. 9 Isolation of 26-23 C2 clones

FIG. 10 Isolation of 22 1C11 clones

FIG. 11 CDR sequences of 26-5F12, 26-23C2 and 22-1C11.

### SEQUENCE LISTING

SEQ ID NO 1: Amino acid 425-436 of Pneumolysin
SEQ ID NO 2: Amino acid 423-438 of Pneumolysin
SEQ ID NO 3: Variable light chain 26-5F12.1
SEQ ID NO 4: Variable heavy chain 26-5F12.1
SEQ ID NO 5: CDR 1 light chain 26-5F12.1
SEQ ID NO 6: CDR 2 light chain 26-5F12.1
SEQ ID NO 7: CDR 3 light chain 26-5F12.1
SEQ ID NO 8: CDR 1 heavy chain 26-5F12.1
SEQ ID NO 9: CDR 2 heavy chain 26-5F12.1
SEQ ID NO 10: CDR 3 heavy chain 26-5F12.1 and 26-23C2.2
SEQ ID NO 11: Pneumolysin sequence
SEQ ID NO 12: Variable light chain 26-23C2.2
SEQ ID NO 13: Variable heavy chain 26-23C2.2
SEQ ID NO 14: CDR 1 light chain 26-23C2.2
SEQ ID NO 15: CDR 2 light chain 26-23C2.2
SEQ ID NO 16: CDR 3 light chain 26-23C2.2
SEQ ID NO 17: CDR 1 heavy chain 26-23C2.2
SEQ ID NO 18: CDR 2 heavy chain 26-23C2.2
SEQ ID NO 19: Variable light chain 22-1C11
SEQ ID NO 20: Variable heavy chain 22-1C11
SEQ ID NO 21: CDR I light chain 22-1C11
SEQ ID NO 22: CDR 2 light chain 22-1C11
SEQ ID NO 23: CDR 3 light chain 22-1C11
SEQ ID NO 24: CDR 1 heavy chain 22-1C11
SEQ ID NO 25: CDR 2 heavy chain 22-1C11
SEQ ID NO 26: CDR 3 heavy chain 22-1C11

### DETAILED DESCRIPTION OF THE INVENTION

### Definitions

Affinity: the strength of binding between receptors and their ligands, for example between an antibody and its antigen.

Avidity: The functional combining strength of an antibody with its antigen which is related to both the affinity of the reaction between the epitopes and paratopes, and the valencies of the antibody and antigen

Amino Acid Residue: An amino acid formed upon chemical digestion (hydrolysis) of a polypeptide at its peptide linkages. The amino acid residues described herein are preferably in the “L” isomeric form. However, residues in the “D” isomeric form can be substituted for any L-amino acid residue, as long as the desired functional property is retained by the polypeptide. NH₂ refers to the free amino group present at the amino terminus of a polypeptide. COOH refers to the free carboxy group present at the carboxy terminus of a polypeptide. In keeping with standard polypeptide, abbreviations for amino acid residues are shown in the following Table of Correspondence:

TABLE OF CORRESPONDENCE

| SYMBOL | | |
|---|---|---|
| 1-Letter | 3-Letter | Amino acid |
| Y | Tyr | tyrosine |
| G | Gly | glycine |
| F | Phe | phenylalanine |
| M | Met | methionine |
| A | Ala | alanine |
| S | Ser | serine |
| I | Ile | isoleucine |
| L | Leu | leucine |
| T | Thr | threonine |
| V | Val | valine |
| P | Pro | proline |
| K | Lys | lysine |
| H | His | histidine |
| Q | Gln | glutamine |
| E | Glu | glutamic acid |
| Z | Glx | Glu and/or Gln |
| W | Trp | tryptophan |
| R | Arg | arginine |
| D | Asp | aspartic acid |
| N | Asn | asparagine |
| B | Asx | Asn and/or Asp |
| C | Cys | cysteine |
| X | Xaa | unknown or other |

It should be noted that all amino acid residue sequences represented herein by formulae have a left-to-right. orientation in the conventional direction of amino terminus to carboxy terminus. In addition, the phrase “amino acid residue” is broadly defined to include the amino acids listed in the Table of Correspondence as well as modified and unusual amino acids. Furthermore, it should be noted that a dash at the beginning or end of an amino acid residue sequence indicates a peptide bond to a further sequence of one or more amino acid residues or a covalent bond to an amino-terminal group such as NH₂ or acetyl or to a carboxy-terminal group such as COOH.

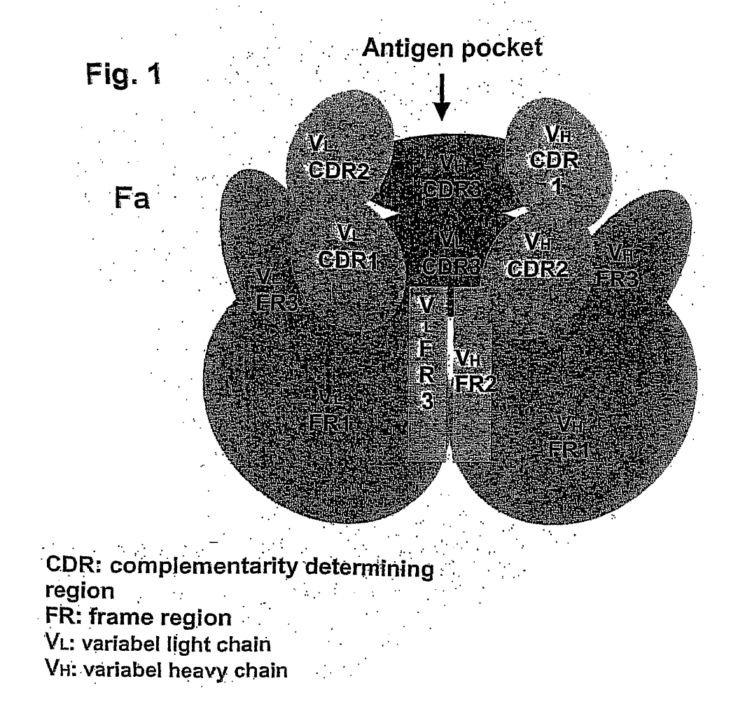

Antibody: The term antibody in its various grammatical forms is used herein to refer to immunoglobulin molecules and immunologically active portions of immunoglobulin molecules of the compositions of this invention, i.e., molecules that contain an antibody combining site or paratope. Exemplary antibody molecules are intact immunoglobulin molecules, substantially intact immunoglobulin molecules and portions of an immunoglobulin molecule, including those portions known in the art as Fab, Fab′, F(ab′)₂ and Fv. A schematic drawing of Fab is shown in FIG. 1. The term “antibody” as used herein is also intended to include human, single chain and humanized antibodies, as well as binding fragments of such antibodies or modified versions of such antibodies, such as multispecific, bispecific and chimeric molecules having at least one antigen binding determinant derived from an antibody molecule.

Antibody Classes Depending on the amino acid sequences of the constant domain of their heavy chains, immunoglobulins can be assigned to different classes. There are at least five (5) major classes of immunoglobulins: IgA, IgD, IgE, IgG and IgM, and several of these may be further divided into subclasses (isotypes), e.g. IgG-1, IgG-2, IgG-3 and IgG-4; IgA-1 and IgA-2. The heavy chains constant domains that correspond to the different classes of immunoglobulins are called alpha (α), delta (δ), epsilon (ε), gamma (γ) and mu (μ), respectively. The light chains of antibodies can be assigned to one of two clearly distinct types, called kappa (K) and lambda (λ), based on the amino sequences of their constant domain. The subunit structures and three-dimensional configurations of different classes of immunoglobulins are well known.

Antibody Combining Site: An antibody combining site is that structural portion of an antibody molecule comprised of a heavy and light chain variable and hypervariable regions that specifically binds (immunoreacts with) an antigen. The term immunoreact in its various forms means specific binding between an antigenic determinant-containing molecule and a molecule containing an antibody combining site such as a whole antibody molecule or a portion thereof. Alternatively, an antibody combining site is known as an antigen binding site.

Anti-haemolytic: Capability to inhibit haemolysis. Here by inhibition of the haemolytic activity of Pneumolysin on erythrocytes.

Base Pair (bp): A partnership of adenine (A) with thymine (T), or of cytosine (C) with guanine (G) in a double stranded DNA molecule. In RNA, uracil (U) is substituted for thymine.

Binding member: a polypeptide that can bind to an epitope on a Streptococcus pneumoniae protein, in particular capable of binding specifically to Pneumolysin.

Binding domain: An antigen binding site which specifically binds an antigen. A binding member may be multispecific and contain two or more binding domains which specifically bind two immunologically distinct antigens.

Chimeric antibody: An antibody in which the variable regions are from one species of animal and the constant regions are from another species of animal. For example, a chimeric antibody can be an antibody having variable regions which derive from a mouse monoclonal antibody and constant regions which are human.

Complementary Bases Nucleotides that normally pair up when DNA or RNA adopts a double stranded configuration.

Complementarity determining region or CDR: Regions in the V-domains of an anti-body that together form the antibody recognizing and binding domain.

Complementary Nucleotide Sequence: A sequence of nucleotides in a singlestranded molecule of DNA or RNA that is sufficiently complementary to that on another single strand to specifically hybridize to it with consequent hydrogen bonding.

Conserved: A nucleotide sequence is conserved with respect to a preselected (reference) sequence if it non-randomly hybridizes to an exact complement of the preselected sequence.

Conservative Substitution: The term conservative substitution as used herein denotes the replacement of an amino acid residue by another, biologically similar residue. Examples of conservative substitutions include the substitution of one hydrophobic residue such as isoleucine, valine, leucine or methionine for another, or the substitution of one polar residue for another, such as the substitution of arginine for lysine, glutamic for aspartic acids, or glutamine for asparagine, and the like. The term conservative substitution also includes the use of a substituted amino acid in place of an unsubstituted parent amino acid provided that molecules having the substituted polypeptide also have the same function.

Constant Region or constant domain or C-domain: Constant regions are those structural portions of an antibody molecule comprising amino acid residue sequences within a given isotype which may contain conservative substitutions therein. Exemplary heavy chain immunoglobulin constant regions are those portions of an immunoglobulin molecule known in the art as CH1, CH2, CH3, CH4 and CH5. An exemplary light chain immunoglobulin constant region is that portion of an immunoglobulin molecule known in the art as C_{L}.

Diabodies: This term refers to a small antibody fragments with two antigen-binding sites, which fragments comprise a heavy chain variable domain (VH) connected to a light chain variable domain (VL) in the same polypeptide chain (VH-VL). By using a linker that is too short to allow pairing between the two domains on the same chain, the domains are forced to pair with the complementary domains of another chain and create two antigen-binding sites. Diabodies are described more fully in, for example, EP 404,097; WO 93/11161; and Hollinger et al., Proc. Natl. Acad. Sci. USA 90: 6444-6448 (1993).

Dissociation constant, Kd: A measure to describe the strength of binding (or affinity or avidity) between receptors and their ligands, for example an antibody and its antigen. The smaller Kd, the stronger binding.

Downstream: Further along a DNA sequence in the direction of sequence transcription or read out, that is travelling in a 3′- to 5′-direction along the non-coding strand of the DNA or 5′- to 3′-direction along the RNA transcript.

Duplex DNA: A double-stranded nucleic acid molecule comprising two strands of substantially complementary polynucleotides held together by one or more hydrogen bonds between each of the complementary bases present in a base pair of the duplex. Because the nucleotides that form a base pair can be either a ribonucleotide base or a deoxyribonucleotide base, the phrase “duplex DNA” refers to either a DNA-DNA duplex comprising two DNA strands (ds DNA), or an RNA-DNA duplex comprising one DNA and one RNA strand.

Fusion Polypeptide: A polypeptide comprised of at least two polypeptides and a linking sequence to operatively link the two polypeptides into one continuous polypeptide. The two polypeptides linked in a fusion polypeptide are typically derived from two independent sources, and therefore a fusion polypeptide comprises two linked polypeptides not normally found linked in nature.

Fv: dual chain antibody fragment containing both a VH and a V,

Gene: A nucleic acid whose nucleotide sequence codes for an RNA or polypeptide. A gene can be either RNA or DNA.

Human antibody framework: A molecule having an antigen binding site and essentially all remaining immunoglobulin-derived parts of the molecule derived from a human immunoglobulin.

Humanised antibody framework: A molecule having an antigen binding site derived from an immunoglobulin from a non-human species, whereas some or all of the remaining immunoglobulin-derived parts of the molecule is derived from a human immunoglobulin. The antigen binding site may comprise: either a complete variable domain from the non-human immunoglobulin fused onto one or more human constant domains; or one or more of the complementarity determining regions (CDRS) grafted onto appropriate human framework regions in the variable domain. In a humanized antibody, the CDRs can be from a mouse monoclonal antibody and the other regions of the antibody are human.

Hybridization: The pairing of substantially complementary nucleotide sequences (strands of nucleic acid) to form a duplex or heteroduplex by the establishment of hydrogen bonds between complementary base pairs. It is a specific, i.e. nonrandom, interaction between two complementary polynucleotides that can be competitively inhibited.

Immunoglobulin: The serum antibodies, including IgG, IgM, IgA, IgE and IgD.

Immunoglobulin isotypes: The names given to the Ig which have different H chains, the names are IgG (IgG_{1,2,3,4}), IgM, IgA (IgA_{1,2}), sigA, IgE, IgD.

Immunologically distinct: The phrase immunologically distinct refers to the ability to distinguish between two polypeptides on the ability of an antibody to specifically bind one of the polypeptides and not specifically bind the other polypeptide.

Individual: A living animal or human in need of susceptible to a condition, in particular an infectious disease” as defined below. The subject is an organism possessing leukocytes capable of responding to antigenic stimulation and growth factor stimulation. In preferred embodiments, the subject is a mammal, including humans and non-human mammals such as dogs, cats, pigs, cows, sheep, goats, horses, rats, and mice. In the most preferred embodiment, the subject is a human.

Infectious disease: a disorder caused by one or more species of Streptococcus, in particular Streptococcus pneumoniae.

Isolated: is used to describe the various binding members, polypeptides and nucleotides disclosed herein, that has been identified and separated and/or recovered from a component of its natural environment. Contaminant components of its natural environment are materials that would typically interfere with diagnostic or therapeutic uses for the polypeptide, and may include enzymes, hormones, and other proteinaceous or non-proteinaceous solutes. In preferred embodiments, the polypeptide will be purified.

Label and indicating means: refer in their various grammatical forms to single atoms and molecules that are either directly or indirectly involved in the production of a detectable signal to indicate the presence of a complex

Monoclonal Antibody: The phrase monoclonal antibody in its various grammatical forms refers to a population of antibody molecules that contains only one species of antibody combining site capable of immunoreacting with a particular antigen. A monoclonal antibody thus typically displays a single binding affinity for any antigen with which it immunoreacts. A monoclonal antibody may contain an antibody molecule having a plurality of antibody combining sites, each immunospecific for a different antigen, e.g., a bispecific monoclonal antibody.

Multimeric: A polypeptide molecule comprising more than one polypeptide. A multimer may be dimeric and contain two polypeptides and a multimer may be trimeric and contain three polypeptides. Multimers may be homomeric and contain two or more identical polypeptides or a multimer may be heteromeric and contain two or more non-identical polypeptides.

Nucleic Acid: A polymer of nucleotides, either single or double stranded.

Nucleotide: A monomeric unit of DNA or RNA consisting of a sugar moiety (pentose), a phosphate, and a nitrogenous heterocyclic base. The base is linked to the sugar moiety via the glycosidic carbon (1′ carbon of the pentose) and that combination of base and sugar is a nucleoside. When the nucleoside contains a phosphate group bonded to the 3′ or 5′ position of the pentose it is referred to as a nucleotide. A sequence of operatively linked nucleotides is typically referred to herein as a “base sequence” or “nucleotide sequence”, and their grammatical equivalents, and is represented herein by a formula whose left to right orientation is in the conventional direction of 5′-terminus to 3′-terminus.

Nucleotide Analog: A purine or pyrimidine nucleotide that differs structurally from A, T, G, C, or U, but is sufficiently similar to substitute for the normal nucleotide in a nucleic acid molecule.

Pneumococcus: is used synonymously with Streptococcus pneumoniae.

Polyclonal antibody: Polyclonal antibodies are a mixture of antibody molecules recognising a specific given antigen, hence polyclonal antibodies may recognise different epitopes within said antigen.

Polynucleotide: A polymer of single or double stranded nucleotides. As used herein “polynucleotide” and its grammatical equivalents will include the full range of nucleic acids. A polynucleotide will typically refer to a nucleic acid molecule comprised of a linear strand of two or more deoxyribonucleotides and/or ribonucleotides. The exact size will depend on many factors, which in turn depends on the ultimate conditions of use, as is well known in the art. The polynucleotides of the present invention include primers, probes, RNA/DNA segments, oligonucleotides or “oligos” (relatively short polynucleotides), genes, vectors, plasmids, and the like.

Polypeptide: The phrase polypeptide refers to a molecule comprising amino acid residues which do not contain linkages other than amide linkages between adjacent amino acid residues.

Receptor: A receptor is a molecule, such as a protein, glycoprotein and the like, that can specifically (non-randomly) bind to another molecule.

Recombinant DNA (rDNA) molecule: A DNA molecule produced by operatively linking two DNA segments. Thus, a recombinant DNA molecule is a hybrid DNA molecule comprising at least two nucleotide sequences not normally found together in nature. rDNA's not having a common biological origin, i.e., evolutionarily different, are said to be “heterologous”.

Specificity: The term specificity refers to the number of potential antigen binding sites which immunoreact with (specifically bind to) a given antigen in a polypeptide. The polypeptide may be a single polypeptide or may be two or more polypeptides joined by disulfide bonding. A polypeptide may be monospecific and contain one or more antigen binding sites which specifically bind an antigen or a polypeptide may be bispecific and contain two or more antigen binding sites which specifically bind two immunologically distinct antigens. Thus, a polypeptide may contain a plurality of antigen binding sites which specifically bind the same or different antigens.

Serotype: Identification of bacteria within species of Streptococcus that consist of many strains differing from one another in a variety of characteristics. Commonly used characteristics defining serotypes are particular antigenic molecules.

Single Chain Antibody or scFv: The phrase single chain antibody refers to a single polypeptide comprising one or more antigen binding sites. Furthermore, although the H and L chains of an Fv fragment are encoded by separate genes, they may be linked either directly or via a peptide, for example a synthetic linker can be made that enables them to be made as a single protein chain (known as single chain antibody, sAb; Bird et al. 1988 Science 242:423-426; and Huston et al. 1988 PNAS 85:5879-5883) by recombinant methods. Such single chain antibodies are also encompassed within the term “antibody”, and may be utilized as binding determinants in the design and engineering of a multispecific binding molecule.

Upstream: In the direction opposite to the direction of DNA transcription, and therefore going from 5′ to 3′ on the non-coding strand, or 3′ to 5′ on the mRNA.

Valency: The term valency refers to the number of potential antigen binding sites, i.e. binding domains, in a polypeptide. A polypeptide may be monovalent and contain one antigen binding site or a polypeptide may be bivalent and contain two antigen binding sites. Additionally, a polypeptide may be tetravalent and contain four antigen binding sites. Each antigen binding site specifically binds one antigen. When a polypeptide comprises more than one antigen binding site, each antigen binding site may specifically bind the same or different antigens. Thus, a polypeptide may contain a plurality of antigen binding sites and therefore be multivalent and a polypeptide may specifically bind the same or different antigens.

V-domain: Variable domain are those structural portions of an antibody molecule comprising amino acid residue sequences forming the antigen binding sites. An exemplary light chain immunoglobulin variable region is that portion of an immunoglobulin molecule known in the art as V_{L}.

V_{L}: Variable domain of the light chain.

V_{H}: Variable domain of the heavy chain.

Vector: A rDNA molecule capable of autonomous replication in a cell and to which a DNA segment, e.g., gene or polynucleotide, can be operatively linked so as to bring about replication of the attached segment. Vectors capable of directing the expression of genes encoding for one or more polypeptides are referred to herein as “expression vectors”. Particularly important vectors allow cloning of cDNA (complementary DNA) from mRNAs produced using reverse transcriptase.

### DESCRIPTION

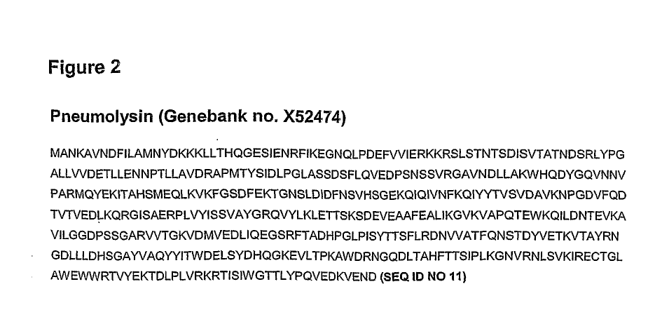

As described above, the present invention relates to binding members, in particular antibodies or fragments thereof capable of specifically recognising and binding to a Streptococcus pneumoniae protein, more specifically to Pneumolysin. The binding members according to the invention are particularly useful in the treatment of diseases caused by Streptococcus pneumoniae, as well as for being employed in diagnostic methods and kits for detecting the bacteria. Pneumolysin is preferably a polypeptide having the amino acid sequence shown in FIG. 2 (SEQ ID NO 11).

Thus, the binding member according to the invention should preferably be immunologically active, for example as an antibody, such as being capable of binding to an antigen and presenting the antigen to immunoactive cells, thereby facilitating phagocytosis of said antigen.

In particular the binding member is an antibody, such as any suitable antibody known in the art, in particular antibodies as defined herein, such as antibodies or immunologically active fragments of antibodies, or single chain antibodies. Antibody molecules are typically Y-shaped molecules whose basic unit consist of four polypeptides, two identical heavy chains and two identical light chains, which are covalently linked together by disulfide bonds. Each of these chains is folded in discrete domains. The C-terminal regions of both heavy and light chains are conserved in sequence and are called the constant regions, also known as C-domains. The N-terminal regions, also known as V-domains, are variable in sequence and are responsible for the antibody specificity. The antibody specifically recognizes and binds to an antigen mainly through six short complementarity-determining regions located in their V-domains (see FIG. 1).

The antibodies according to the invention are especially useful, since they have a strong affinity towards Pneumolysin.

Accordingly, the binding members according to the invention have a binding domain having a dissociation constant K_{d} for Pneumolysin which is less than 1×10⁻⁶ M. More preferably the dissociation constant K_{d} for Pneumolysin is less than 1×10⁻⁷M, more preferably less than 1×10⁻⁸M, more preferably less than 5×10⁻⁸M, more preferably less than 1×10⁻⁹M, more preferably less than 5×10⁻⁹M, more preferably less than 1×10⁻¹⁰M.

The affinity of the binding member towards the Pneumolysin is preferably measured as described in Example 4.

The binding member is preferably an isolated binding member as defined above, and more preferably an isolated, pure binding member.

### Anti-Haemolytic Activity

It is further contemplated that binding members having anti haemolytic activity are particular suitable in the treatment of diseases caused by Streptococcus pneumoniae. With out being bound by the theory it is believed that binding of an anti-haemolytic binding member to Pneumolysin prevents the attachment of Pneumolysin to the membrane of the target cell. In vitro functional assay is prefereably performed as described in example 2 and 3.

It is preferred that the binding member according to the invention is capable of inhibiting haemolysis at least 50% at a concentration of 4000 ng/ml in an assay as described in example 3. More preferably the binding member inhibts haemolysis by at least 60% such as 80, such as 85, most preferably such as 90% at a concentration of 4000 ng/ml in an assay as described in example 3.

Most preferred the binding member according to the invention is capable of inhibiting haemolysis at least 50% at a concentration of 160 ng/ml in an assay as described in example 3. More preferably the binding member inhibits haemolysis by at least 60% such as 80, such as 85, most preferably 90% at a concentration of 160 ng/ml in an assay as described in example 3.

### Complementarity-Determining Regions

Without being bound by theory it is believed that the high binding strength and/or anti-haemolytic activity is caused by incorporating into the binding domain an amino acid sequence having one or more of the following motifs of the sequences shown below.

In an embodiment the binding domain comprises at least one of the amino acid sequence sets selected from the group of:
the amino acid sequence sets SEQ ID NO 5 or a homologue thereof, SEQ ID NO 6 or a homologue thereof, and SEQ ID NO 7 or a homologue thereof, or
the amino acid sequence sets SEQ ID NO 14 or a homologue thereof, SEQ ID NO 15 or a homologue thereof, and SEQ ID NO 16 or a homologue thereof, or
   preferably, the binding domain comprises at least one of the amino acid sequence sets selected from the group of:
the amino acid sequence sets SEQ ID NO 8 or a homologue thereof, SEQ ID NO 9 or a homologue thereof, and SEQ ID NO 10 or a homologue thereof.
the amino acid sequence sets SEQ ID NO 17 or a homologue thereof, SEQ ID NO 18 or a homologue thereof, and SEQ ID NO 10.

In the amino acid sequence sets above, the amino acid sequences are preferably arranged in the binding domain as CDR1, CDR2 and CDR3, i.e. spaced apart by other amino acid sequences.

More specifically the binding domain preferably comprises a CDR1 region comprising a sequence selected from SEQ ID NO 5 and SEQ ID NO 8 or a homologue thereof, and/or the binding domain preferably comprises a CDR2 region comprising a sequence selected from SEQ ID NO 6 and SEQ ID NO 9 or a homologue thereof, and/or the binding domain preferably comprises a CDR3 region comprising a sequence selected from SEQ ID NO 7 and SEQ ID NO 10 or a homologue thereof.

Alternatively the binding domain preferably comprises a CDR1 region comprising a sequence selected from SEQ ID NO 14 and SEQ ID NO 17 or a homologue thereof, and/or the binding domain preferably comprises a CDR2 region comprising a sequence selected from SEQ ID NO 15 and SEQ ID NO 18 or a homologue thereof, and/or the binding domain preferably comprises a CDR3 region comprising a sequence selected from SEQ ID NO 16 and SEQ ID NO 10 or a homologue thereof.

The findings of the applicant described herein suggest that the sequence of the variable heavy chain may be important for haemolytic activity. Thus preferred embodiments include binding domains comprising one or more of the sequences sequence selected from the group of; SEQ ID NO 8, SEQ ID NO 9, SEQ ID NO 17, SEQ ID NO 18 and SEQ ID NO 10 or a homologue thereof. Especially preferred, is a binding domain comprising SEQ ID NO 9 or SEQ ID NO 18 or a homologue thereof. Mostly preferred is a binding domain comprising SEQ ID NO 10 or a homologue thereof.

Thus it is particularly preferably, that the variable part of the binding domain comprises a sequence selected from SEQ ID NO 3 and SEQ ID NO 4 or a homologue thereof, wherein a homologue is as defined elsewhere herein.

Alternatively, the variable part of the binding domain comprises a sequence selected from SEQ ID NO 12 and SEQ ID NO 13 or a homologue thereof, wherein a homologue is as defined elsewhere herein.

In preferred specific embodiment the variable light chain of the binding domain comprises a sequence selected from SEQ ID NO 3 and SEQ ID NO 12 or/and most preferably the variable heavy chain of the binding domain comprises a sequence selected from SEQ ID NO 4 and SEQ ID NO 13.

The homology of any one of the homologues described above preferably confers the binding domain comprising one or more homologues with dissociation constant K_{d} for Pneumolysin as defined above.

### Identity and Homology

The term “identity” shall be construed to mean the percentage of amino acid residues in the candidate sequence that are identical with the residue of a corresponding sequence to which it is compared, after aligning the sequences and introducing gaps, if necessary to achieve the maximum percent identity for the entire sequence, and not considering any conservative substitutions as part of the sequence identity. Neither N- or C-terminal extensions nor insertions shall be construed as reducing identity or homology. Methods and computer programs for the alignment are well known in the art. Sequence identity may be measured using sequence analysis software (e.g., Sequence Analysis Software Package, Genetics Computer Group, University of Wisconsin Biotechnology Center, 1710 University Ave., Madison, Wis. 53705). This software matches similar sequences by assigning degrees of homology to various substitutions, deletions, and other modifications.

A homologue of one or more of the sequences specified herein may vary in one or more amino acids as compared to the sequences defined, but is capable of performing the same function, i.e. a homologue may be envisaged as a functional equivalent of a predetermined sequence.

As described above a homologue of any of the predetermined sequences herein may be defined as:
i) homologues comprising an amino acid sequence capable of recognising an antigen also being recognised by the predetermined amino acid sequence, and/or
ii) homologues comprising an amino acid sequence capable of binding selectively to an antigen, wherein said antigen is also bound selectively by a predetermined sequence, and/or
iii) homologues having a substantially similar or higher binding affinity to Pneumolysin as a binding domain comprising a predetermined sequence, such as SEQ ID NO 3, 4 12 and 13.

Examples of homologues comprises one or more conservative amino acid substitutions including one or more conservative amino acid substitutions within the same group of predetermined amino acids, or a plurality of conservative amino acid substitutions, wherein each conservative substitution is generated by substitution within a different group of predetermined amino acids.

Homologues may thus comprise conservative substitutions independently of one another, wherein at least one glycine (Gly) of said homologue is substituted with an amino acid selected from the group of amino acids consisting of Ala, Val, Leu, and Ile, and independently thereof, homologues, wherein at least one of said alanines (Ala) of said homologue thereof is substituted with an amino acid selected from the group of amino acids consisting of Gly, Val, Leu, and Ile, and independently thereof, homologues, wherein at least one valine (Val) of said homologue thereof is substituted with an amino acid selected from the group of amino acids consisting of Gly, Ala, Leu, and Ile, and independently thereof, homologues thereof, wherein at least one of said leucines (Leu) of said homologue thereof is substituted with an amino acid selected from the group of amino acids consisting of Gly, Ala, Val, and Ile, and independently thereof, homologues thereof, wherein at least one isoleucine (Ile) of said homologues thereof is substituted with an amino acid selected from the group of amino acids consisting of Gly, Ala, Val and Leu, and independently thereof, homologues thereof wherein at least one of said aspartic acids (Asp) of said homologue thereof is substituted with an amino acid selected from the group of amino acids consisting of Glu, Asn, and Gln, and independently thereof, homologues thereof, wherein at least one of said phenylalanines (Phe) of said homologues thereof is substituted with an amino acid selected from the group of amino acids consisting of Tyr, Trp, His, Pro, and preferably selected from the group of amino acids consisting of Tyr and Trp, and independently thereof, homologues thereof, wherein at least one of said tyrosines (Tyr) of said homologues thereof is substituted with an amino acid selected from the group of amino acids consisting of Phe, Trp, His, Pro, preferably an amino acid selected from the group of amino acids consisting of Phe and Trp, and independently thereof, homologues thereof, wherein at least one of said arginines (Arg) of said fragment is substituted with an amino acid selected from the group of amino acids consisting of Lys and His, and independently thereof, homologues thereof, wherein at least one lysine (Lys) of said homologues thereof is substituted with an amino acid selected from the group of amino acids consisting of Arg and His, and independently thereof, homologues thereof, wherein at least one of said aspargines (Asn) of said homologues thereof is substituted with an amino acid selected from the group of amino acids consisting of Asp, Glu, and Gln, and independently thereof, homologues thereof, wherein at least one glutamine (Gln) of said homologues thereof is substituted with an amino acid selected from the group of amino acids consisting of Asp, Glu, and Asn, and independently thereof, homologues thereof, wherein at least one proline (Pro) of said homologues thereof is substituted with an amino acid selected from the group of amino acids consisting of Phe, Tyr, Trp, and His, and independently thereof, homologues thereof, wherein at least one of said cysteines (Cys) of said homologues thereof is substituted with an amino acid selected from the group of amino acids consisting of Asp, Glu, Lys, Arg, His, Asn, Gln, Ser, Thr, and Tyr.

Conservative substitutions may be introduced in any position of a preferred predetermined sequence. It may however also be desirable to introduce non-conservative substitutions, particularly, but not limited to, a non-conservative substitution in any one or more positions.

A non-conservative substitution leading to the formation of a functionally equivalent homologue of the sequences herein would for example i) differ substantially in polarity, for example a residue with a non-polar side chain (Ala, Leu, Pro, Trp, Val, Ile, Leu, Phe or Met) substituted for a residue with a polar side chain such as Gly, Ser, Thr, Cys, Tyr, Asn, or Gln or a charged amino acid such as Asp, Glu, Arg, or Lys, or substituting a charged or a polar residue for a non-polar one; and/or ii) differ substantially in its effect on polypeptide backbone orientation such as substitution of or for Pro or Gly by another residue; and/or iii) differ substantially in electric charge, for example substitution of a negatively charged residue such as Glu or Asp for a positively charged residue such as Lys, His or Arg (and vice versa); and/or iv) differ substantially in steric bulk, for example substitution of a bulky residue such as His, Trp, Phe or Tyr for one having a minor side chain, e.g. Ala, Gly or Ser (and vice versa).

Substitution of amino acids may in one embodiment be made based upon their hydrophobicity and hydrophilicity values and the relative similarity of the amino acid side-chain substituents, including charge, size, and the like. Exemplary amino acid substitutions which take various of the foregoing characteristics into consideration are well known to those of skill in the art and include: arginine and lysine; glutamate and aspartate; serine and threonine; glutamine and asparagine; and valine, leucine and isoleucine.

In an embodiment the binding domain comprises a homologue having an amino acid sequence at least 60% identical to a sequence selected from SEQ ID NO 5, 6, 7, 8, 9, 10, 14, 15, 16, 17 and 18. In a preferred embodiment the binding domain comprises a homologue having an amino acid sequence at least 60% identical to a sequence selected from SEQ ID NO 3, 4 12 and 13.

More preferably the homologue is at least 65%, such as at least 70% identical, such as at least 75% identical, such as at least 80% identical, such as at least 85% identical, such as at least 90% identical, such as at least 95% identical, such as at least 98% identical to a sequence selected from selected from SEQ ID NO 5, 6, 7, 8, 9, 10, 14, 15, 16, 17 and 18 or preferably SEQ ID NO 3, 4 12 and 13.

In a more preferred embodiment the percentages mentioned above relates to the identity of the sequence of a homologue as compared to a sequence selected from SEQ ID NO 3, 4 12 and 13.

### Epitopes

The anti-haemolytic binding member according to the present invention preferably recognize and bind to an epitope also recognized by an antibody having a variable part comprising a sequence selected from the group of SEQ ID NO 3, 4, 12 or 13.

In an embodiment the binding domain of the anti-haemolytic binding member, recognise an epitope in the N-terminal part of Pneumolysin. Preferably within the N-terminal part corresponding to amino acid 1-436 of Pneumolysin as identified by SEQ ID NO 11. It is further preferred that the epitope recognized by the binding domain is within amino acid 50-436, or preferably amino acid 100-436 of Pneumolysin as identified by SEQ ID NO 11. In specific embodiment the epitope recognized by the binding member is with in amino acid 200-435 or 300-435 of Pneumolysin as identified by SEQ ID NO 11.

The binding domain of the binding member of the invention preferably recognise an epitope comprised by the amino acid sequence identified by SEQ ID NO: 27. In a preferred embodiment the binding domain recognises an epitope comprised by SEQ ID NOs 28, 29, 30 and 31 more preferably an epitope comprised by SEQ ID 29 and 30.

It is further preferred that the epitope recognized by the binding domain is within amino acid 400-438, or preferably amino acid 420-436 of Pneumolysin as identified by SEQ ID NO 11. In specific embodiment the epitope recognized by the binding member is with in amino acid 422-436 or 425-436 of Pneumolysin as identified by SEQ ID NO 11.

### Serotypes

As described above, 90 different serotypes of Streptococcus pneumoniae have been identified. It is preferred that the binding member according to this invention is capable of binding Pneumolysin from two or more different Pneumococcus serotypes, such as from three or more different Pneumococcus serotypes, such as from four or more different Pneumococcus serotypes, such as from five or more different Pneumococcus serotypes. Most preferably the binding member according to the invention is capable of recognising and binding Pneumococcus from essentially all serotypes.

### Monoclonal/Polyclonal Antibodies

In one embodiment of the invention, the binding member is an antibody, wherein the antibody may be a polyclonal or a monoclonal antibody derived from a mammal or mixtures of monoclonal antibodies. In a preferred embodiment the binding member is a monoclonal antibody or a fragment thereof. The antibody may be any kind of antibody; however it is preferably an IgG antibody. More preferably the antibody is an IgG1 antibody or a fragment thereof.

Monoclonal antibodies (Mab's) are antibodies, wherein every antibody molecule is similar and thus recognises the same epitope. Monoclonal antibodies are in general produced by a hybridoma cell line. Methods of making monoclonal antibodies and antibody-synthesizing hybridoma cells are well known to those skilled in the art. Antibody-producing hybridomas may for example be prepared by fusion of an antibody-producing B lymphocyte with an immortalized cell line.

A monoclonal antibody can be produced by the following steps. In all procedures, an animal is immunized with an antigen such as a protein (or peptide thereof) as described above for preparation of a polyclonal antibody. The immunization is typically accomplished by administering the immunogen to an immunologically competent mammal in an immunologically effective amount, i.e., an amount sufficient to produce an immune response. Preferably, the mammal is a rodent such as a rabbit, rat or mouse. The mammal is then maintained on a booster schedule for a time period sufficient for the mammal to generate high affinity antibody molecules as described. A suspension of antibody-producing cells is removed from each immunized mammal secreting the desired antibody. After a sufficient time to generate high affinity antibodies, the animal (e.g., mouse) is sacrificed and antibody-producing lymphocytes are obtained from one or more of the lymph nodes, spleens and peripheral blood. Spleen cells are preferred, and can be mechanically separated into individual cells in a physiological medium using methods well known to one of skill in the art. The antibody-producing cells are immortalized by fusion to cells of a mouse myeloma line. Mouse lymphocytes give a high percentage of stable fusions with mouse homologous myelomas, however rat, rabbit and frog somatic cells can also be used. Spleen cells of the desired antibody-producing animals are immortalized by fusing with myeloma cells, generally in the presence of a fusing agent such as polyethylene glycol. Any of a number of myeloma cell lines suitable as a fusion partner are used with to standard techniques, for example, the P3-NS1/1-Ag4-1, P3-x63-Ag8.653 or Sp2/O-Ag14 myeloma lines, available from the American Type Culture Collection (ATCC), Rockville, Md.

Monoclonal antibodies can also be generated by other methods well known to those skilled in the art of recombinant DNA technology. An alternative method, referred to as the “combinatorial antibody display” method, has been developed to identify and isolate antibody fragments having a particular antigen specificity, and can be utilized to produce monoclonal antibodies.

Polyclonal antibodies is a mixture of antibody molecules recognising a specific given antigen, hence polyclonal antibodies may recognise different epitopes within said antigen. In general polyclonal antibodies are purified from serum of a mammal, which previously has been immunized with the antigen. Polyclonal antibodies may for example be prepared by any of the methods described in Antibodies: A Laboratory Manual, By Ed Harlow and David Lane, Cold Spring Harbor Laboratory Press, 1988. Polyclonal antibodies may be derived from any suitable mammalian species, for example from mice, rats, rabbits, donkeys, goats, and sheep.

### Specificity

The binding member may be monospecific towards Pneumolysin, wherein specificity towards Pneumolysin means that the binding member immunoreacts with Pneumolysin. In another embodiment, the binding member is bispecific or multispecific having at least one portion being specific towards Pneumolysin.

### Monovalent Antibodies

The monospecific binding member may be monovalent, i.e. having only one binding domain.

For a monovalent antibody, the immunoglobulin constant domain amino acid residue sequences comprise the structural portions of an antibody molecule known in the art as CH1, CH2, CH3 and CH4. Preferred are those binding members which are known in the art as C_{L}. Preferred C_{L} polypeptides are selected from the group consisting of Cₖₐₚₚₐ and C_{lambda}.

Furthermore, insofar as the constant domain can be either a heavy or light chain constant domain (C_{H} or C_{L}, respectively), a variety of monovalent binding member compositions are contemplated by the present invention. For example, light chain constant domains are capable of disulfide bridging to either another light chain constant domain, or to a heavy chain constant domain. In contrast, a heavy chain constant domain can form two independent disulfide bridges, allowing for the possibility of bridging to both another heavy chain and to a light chain, or to form polymers of heavy chains.

Thus, in another embodiment, the invention contemplates a composition comprising a monovalent polypeptide wherein the constant chain domain C has a cysteine residue capable of forming at least one disulfide bridge, and where the composition comprises at least two monovalent polypeptides covalently linked by said disulfide bridge.

In preferred embodiments, the constant chain domain C can be either C_{L} or C_{H}. Where C is C_{L}, the C_{L} polypeptide is preferably selected from the group consisting of Cₖₐₚₚₐ and C_{lambda}.

In another embodiment, the invention contemplates a binding member composition comprising a monovalent polypeptide as above except where C is C_{L} having a cysteine residue capable of forming a disulfide bridge, such that the composition contains two monovalent polypeptides covalently linked by said disulfide bridge.

### Multivalent

In another embodiment of the invention the binding member is a multivalent binding member having at least two binding domains. The binding domains may have specificity for the same ligand or for different ligands.

### Multispecificity, Including Bispecificity

In a preferred embodiment the present invention relates to multispecific binding members, which have affinity for and are capable of binding at least two different entities. Multispecific binding members can include bispecific binding members.

In one embodiment the multispecific molecule is a bispecific antibody (BsAb), which carries at least two different binding domains, at least one of which is of antibody origin.

A bispecific molecule of the invention can also be a single chain bispecific molecule, such as a single chain bispecific antibody, a single chain bispecific molecule comprising one single chain antibody and a binding domain, or a single chain bispecific molecule comprising two binding domains. Multispecific molecules can also be single chain molecules or may comprise at least two single chain molecules.

The multispecific, including bispecific, antibodies may be produced by any suitable manner known to the person skilled in the art.

The traditional approach to generate bispecific whole antibodies was to fuse two hybridoma cell lines each producing an antibody having the desired specificity. Because of the random association of immunoglobulin heavy and light chains, these hybrid hybridomas produce a mixture of up to 10 different heavy and light chain combinations, only one of which is the bispecific antibody. Therefore, these bispecific antibodies have to be purified with cumbersome procedures, which considerably decrease the yield of the desired product.

Alternative approaches include in vitro linking of two antigen specificities by chemical cross-linking of cysteine residues either in the hinge or via a genetically introduced C-terminal Cys as described above. An improvement of such in vitro assembly was achieved by using recombinant fusions of Fab's with peptides that promote formation of heterodimers. However, the yield of bispecific product in these methods is far less than 100%.

A more efficient approach to produce bivalent or bispecific antibody fragments, not involving in vitro chemical assembly steps, was described by Holliger et al. (1993). This approach takes advantage of the observation that scFv's secreted from bacteria are often present as both monomers and dimers. This observation suggested that the V_{H} and V_{L} of different chains could pair, thus forming dimers and larger complexes. The dimeric antibody fragments, also named “diabodies” by Hollinger et al., are in fact small bivalent antibody fragments that assembled in vivo. By linking the V_{H} and V_{L} of two different antibodies 1 and 2, to form “cross-over” chains V_{H} 1V_{L} 2 and V_{H} 2-V_{L} 1, the dimerisation process was shown to reassemble both antigen-binding sites. The affinity of the two binding sites was shown to be equal to the starting scFv's, or even to be 10-fold increased when the polypeptide linker covalently linking V_{H} and V_{L} was removed, thus generating two proteins each consisting of a V_{H} directly and covalently linked to a V_{L} not pairing with the V_{H}. This strategy of producing bispecific antibody fragments was also described in several patent applications. Patent application WO 94/09131 (SCOTGEN LTD; priority date Oct. 15, 1992) relates to a bispecific binding protein in which the binding domains are derived from both a V_{H} and a V_{L} region either present at two chains or linked in an scFv, whereas other fused antibody domains, e.g. C-terminal constant domains, are used to stabilise the dimeric constructs. Patent application WO 94/13804 (CAMBRIDGE ANTIBODY TECHNOLOGY/MEDICAL RESEARCH COUNCIL; first priority date Dec. 4, 1992) relates to a polypeptide containing a V_{H} and a V_{L} which are incapable of associating with each other, whereby the V-domains can be connected with or without a linker.

Mallender and Voss, 1994 (also described in patent application WO 94/13806; DOW CHEMICAL CO; priority date Dec. 11, 1992) reported the in vivo production of a single-chain bispecific antibody fragment in E. coli. The bispecificity of the bivalent protein was based on two previously produced monovalent scFv molecules possessing distinct specificities, being linked together at the genetic level by a flexible polypeptide linker. Traditionally, whenever single-chain antibody fragments are referred to, a single molecule consisting of one heavy chain linked to one (corresponding) light chain in the presence or absence of a polypeptide linker is implicated. When making bivalent or bispecific antibody fragments through the “diabody” approach (Holliger et al., (1993) and patent application WO 94/09131) or by the “double scFv” approach (Mallender and Voss, 1994 and patent application WO 94/13806), again the V_{H} is linked to a (the corresponding) VL.

The multispecific molecules described above can be made by a number of methods. For example, all specificities can be encoded in the same vector and expressed and assembled in the same host cell. This method is particularly useful where the multispecific molecule is a mAb X mAb, mAb X Fab, Fab X F(ab′)₂ or ligand X Fab fusion protein. Various other methods for preparing bi- or multivalent antibodies are described for example described in U.S. Pat. Nos. 5,260,203; 5,455,030; 4,881,175; 5,132,405; 5,091,513; 5,476,786; 5,013,653; 5,258,498; and 5,482,858. By using a bispecific or multispecific binding member according to the invention the invention offers several advantages as compared to monospecific/monovalent binding members.

A bispecific/multispecific binding member has a first binding domain capable of specifically recognising and binding a Streptococcus protein, in particular Pneumolysin, whereas the other binding domain(s) may be used for other purposes:

In one embodiment at least one other binding domain is used for binding to a Streptococcus protein, such as binding to another epitope on the same Streptococcus protein as compared to the first binding domain. Thereby specificity for the Streptococcus species may be increased as well as increase of avidity of the binding member.

In another embodiment the at least one other binding domain may be used for specifically binding a mammalian cell, such as a human cell. It is preferred that the at least other binding domain is capable of binding an immunoactive cell, such as a leucocyte, a macrophage, a lymphocyte, a basophilic cell, and/or an eosinophilic cell, in order to increase the effect of the binding member in a therapeutic method. This may be accomplished by establishing that the at least one other binding domain is capable of specifically binding a mammalian protein, such as a human protein, such as a protein selected from any of the cluster differentiation proteins (CD), in particular CD64 and/or CD89. A method for producing bispecific antibodies having CD64 specificity is described in U.S. Pat. No. 6,071,517 to Medarex, Inc.

An “effector cell” as used herein refers to an immune cell which is a leukocyte or a lymphocyte. Specific effector cells express specific Fc receptors and carry out specific immune functions. For example, monocytes, macrophages, neutrophils, eosinophils, and lymphocytes which express CD89 receptor are involved in specific killing of target cells and presenting antigens to other components of the immune system, or binding to cells that present antigens.

### Humanised Antibody Framework

It is not always desirable to use non-human antibodies for human therapy, since the non-human “foreign” epitopes may elicit immune response in the individual to be treated. To eliminate or minimize the problems associated with non-human antibodies, it is desirable to engineer chimeric antibody derivatives, i.e., “humanized” anti-body molecules that combine the non-human Fab variable region binding determinants with a human constant region (Fc). Such antibodies are characterized by equivalent antigen specificity and affinity of the monoclonal and polyclonal antibodies described above, and are less immunogenic when administered to humans, and therefore more likely to be tolerated by the individual to be treated.

Accordingly, in one embodiment the binding member has a binding domain carried on a humanised antibody framework, also called a humanised antibody.

Humanised antibodies are in general chimeric antibodies comprising regions derived from a human antibody and regions derived from a non-human antibody, such as a rodent antibody. Humanisation (also called Reshaping or CDR-grafting) is a well-established technique for reducing the immunogenicity of monoclonal antibodies (mAbs) from xenogeneic sources (commonly rodent), increasing the homology to a human immunoglobulin, and for improving their activation of the human immune system. Thus, humanized antibodies are typically human antibodies in which some CDR residues and possibly some framework residues are substituted by residues from analogous sites in rodent antibodies.

It is further important that humanized antibodies retain high affinity for the antigen and other favourable biological properties. To achieve this goal, according to a preferred method, humanized antibodies are prepared by a process of analysis of the parental sequences and various conceptual humanized products using three-dimensional models of the parental and humanized sequences. Three-dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of certain residues in the functioning of the candidate immunoglobulin sequence, i.e., the analysis of residues that influence the ability of the candidate immunoglobulin to bind its antigen. In this way, FR residues can be selected and combined from the recipient and import sequences so that the desired antibody characteristic, such as increased affinity for the target antigen(s), is maximized, although it is the CDR residues that directly and most substantially influence antigen binding.

One method for humanising MAbs related to production of chimeric antibodies in which an antigen binding site comprising the complete variable domains of one anti-body are fused to constant domains derived from a second antibody, preferably a human antibody. Methods for carrying out such chimerisation procedures are for example described in EP-A-0 120 694 (Celltech Limited), EP-A-0 125 023 (Genentech Inc.), EP-A-0 171 496 (Res. Dev. Corp. Japan), EP-A-0173494 (Stanford University) and EP-A-0 194 276 (Celltech Limited). A more complex form of humanisation of an antibody involves the re-design of the variable region domain so that the amino acids constituting the non-human antibody binding site are integrated into the framework of a human antibody variable region (Jones et al., 1986).

The humanized antibody of the present invention may be made by any method capable of replacing at least a portion of a CDR of a human antibody with a CDR derived from a non-human antibody. Winter describes a method which may be used to prepare the humanized antibodies of the present invention (UK Patent Application GB 2188638A, filed on Mar. 26, 1987), the contents of which is expressly incorporated by reference. The human CDRs may be replaced with non-human CDRs using oligonucleotide site-directed mutagenesis as described in the examples below.

As an example the humanized antibody of the present invention may be made as described in the brief explanation below. The humanized antibodies of the present invention may be produced by the following process:
(a) constructing, by conventional techniques, an expression vector containing an operon with a DNA sequence encoding an antibody heavy chain in which the CDRs and such minimal portions of the variable domain framework region that are required to retain antibody binding specificity are derived from a non-human immunoglobulin, and the remaining parts of the antibody chain are derived from a human immunoglobulin, thereby producing the vector of the invention;
(b) constructing, by conventional techniques, an expression vector containing an operon with a DNA sequence encoding a complementary antibody light chain in which the CDRs and such minimal portions of the variable domain framework region that are required to retain donor antibody binding specificity are derived from a non-human immunoglobulin, and the remaining parts of the antibody chain are derived from a human immunoglobulin, thereby producing the vector of the invention;
(c) transfecting the expression vectors into a host cell by conventional techniques to produce the transfected host cell of the invention; and
(d) culturing the transfected cell by conventional techniques to produce the humanised antibody of the invention.

The host cell may be cotransfected with the two vectors of the invention, the first vector containing an operon encoding a light chain derived polypeptide and the second vector containing an operon encoding a heavy chain derived polypeptide. The two vectors contain different selectable markers, but otherwise, apart from the anti-body heavy and light chain coding sequences, are preferably identical, to ensure, as far as possible, equal expression of the heavy and light chain polypeptides. Alternatively, a single vector may be used, the vector including the sequences encoding both the light and the heavy chain polypeptides. The coding sequences for the light and heavy chains may comprise cDNA or genomic DNA or both.

The host cell used to express the altered antibody of the invention may be either a bacterial cell such as Escherichia coli, or a eukaryotic cell. In particular a mammalian cell of a well defined type for this purpose, such as a myeloma cell or a Chinese hamster ovary cell may be used.

The general methods by which the vectors of the invention may be constructed, transfection methods required to produce the host cell of the invention and culture methods required to produce the antibody of the invention from such host cells are all conventional techniques. Likewise, once produced, the humanized antibodies of the invention may be purified according to standard procedures as described below.

### Human Antibody Framework

In a more preferred embodiment the invention relates to a binding member, wherein the binding domain is carried by a human antibody framework, i.e. wherein the antibodies have a greater degree of human peptide sequences than do humanised antibodies.

Human mAb antibodies directed against human proteins can be generated using transgenic mice carrying the complete human immune system rather than the mouse system. Splenocytes from these transgenic mice immunized with the antigen of interest are used to produce hybridomas that secrete human mAbs with specific affinities for epitopes from a human protein (see, e.g., Wood et al. International Application WO 91/00906, Kucherlapati et al. PCT publication WO 91/10741; Lonberg et al., International Application WO 92/03918; Kay et al. International Application 92/03917; Lonberg, N. et al. 1994 Nature 368:856-859; Green, L. L. et al. 1994 Nature Genet. 7:13-21; Morrison, S. L. et al. 1994 Proc. Natl. Acad. Sci. USA 81:6851-6855; Bruggeman et al. 1993 Year Immunol 7:33-40; Tuaillon et al. 1993 PNAS 90:3720-3724; Bruggeman et al. 1991 Eur J Immunol 21:1323-1326).

Such transgenic mice are available from Abgenix, Inc., Fremont, Calif., and Medarex, Inc., Annandale, N.J. It has been described that the homozygous deletion of the antibody heavy-chain joining region (IH) gene in chimeric and germ-line mutant mice results in complete inhibition of endogenous antibody production. Transfer of the human germ-line immunoglobulin gene array in such germ-line mutant mice will result in the production of human antibodies upon antigen challenge. See, e.g., Jakobovits et al., Proc. Natl. Acad. Sci. USA 90:2551 (1993); Jakobovits et al., Nature 362:255-258 (1993); Bruggermann et al., Year in Immunol. 7:33 (1993); and Duchosal et al. Nature 355:258 (1992). Human antibodies can also be derived from phage-display libraries (Hoogenboom et al., J. Mol. Biol. 227: 381 (1991); Marks et al., J. Mol. Biol. 222:581-597 (1991); Vaughan, et al., Nature Biotech 14:309 (1996)).

### Fragments

In one embodiment of the invention the binding member is a fragment of an antibody, preferably an antigen binding fragment or a variable region. Examples of anti-body fragments useful with the present invention include Fab, Fab′, F(ab′) 2 and Fv fragments. Papain digestion of antibodies produces two identical antigen binding fragments, called the Fab fragment, each with a single antigen binding site, and a residual “Fc” fragment, so-called for its ability to crystallize readily. Pepsin treatment yields an F(ab′)₂ fragment that has two antigen binding fragments which are capable of cross-linking antigen, and a residual other fragment (which is termed pFc′). Additional fragments can include diabodies, linear antibodies, single-chain antibody molecules, and multispecific antibodies formed from antibody fragments.

The antibody fragments Fab, Fv and scFv differ from whole antibodies in that the antibody fragments carry only a single antigen-binding site. Recombinant fragments with two binding sites have been made in several ways, for example, by chemical cross-linking of cysteine residues introduced at the C-terminus of the VH of an Fv (Cumber et al., 1992), or at the C-terminus of the VL of an scFv (Pack and Pluckthun, 1992), or through the hinge cysteine residues of Fab's (Carter et al., 1992).

Preferred antibody fragments retain some or essential all the ability of an antibody to selectively binding with its antigen or receptor. Some preferred fragments are defined as follows:
(1) Fab is the fragment that contains a monovalent antigen-binding fragment of an antibody molecule. A Fab fragment can be produced by digestion of whole anti-body with the enzyme papain to yield an intact light chain and a portion of one heavy chain.
(2) Fab′ is the fragment of an antibody molecule and can be obtained by treating whole antibody with pepsin, followed by reduction, to yield an intact light chain and a portion of the heavy chain. Two Fab′ fragments are obtained per antibody molecule. Fab′ fragments differ from Fab fragments by the addition of a few residues at the carboxyl terminus of the heavy chain CH1 domain including one or more cysteines from the antibody hinge region.
(3) (Fab′)₂ is the fragment of an antibody that can be obtained by treating whole antibody with the enzyme pepsin without subsequent reduction. F(ab′)₂ is a dimer of two Fab′ fragments held together by two disulfide bonds.
(4) Fv is the minimum antibody fragment that contains a complete antigen recognition and binding site. This region consists of a dimer of one heavy and one light chain variable domain in a tight, non-covalent association (V_{H}-V_{L} dimer). It is in this configuration that the three CDRs of each variable domain interact to define an antigen binding site on the surface of the V_{H}-V_{L} dimer. Collectively, the six CDRs confer antigen binding specificity to the antibody. However, even a single variable domain (or half of an Fv comprising only three CDRs specific for an antigen) has the ability to recognize and bind antigen, although at a lower affinity than the entire binding site.

In one embodiment of the present invention the antibody is a single chain antibody (“SCA”), defined as a genetically engineered molecule containing the variable region of the light chain, the variable region of the heavy chain, linked by a suitable polypeptide linker as a genetically fused single chain molecule. Such single chain anti-bodies are also referred to as “single-chain Fv” or “sFv” antibody fragments. Generally, the Fv polypeptide further comprises a polypeptide linker between the V_{H} and VL domains that enables the sFv to form the desired structure for antigen binding.

The antibody fragments according to the invention may be produced in any suitable manner known to the person skilled in the art. Several microbial expression systems have already been developed for producing active antibody fragments, e.g. the production of Fab in various hosts, such as E. coli (Better et al., 1988, Skerra and Pluckthun, 1988, Carter et al., 1992), yeast (Horwitz et al., 1988), and the filamentous fungus Trichoderma reesei (Nyyssonen et al., 1993) has been described. The recombinant protein yields in these alternative systems can be relatively high (1-2g/l for Fab secreted to the periplasmic space of E. coli in high cell density fermentation, see Carter et al., 1992), or at a lower level, e.g. about 0.1 mg/l for Fab in yeast in fermenters (Horwitz et al., 1988), and 150 mg/l for a fusion protein CBHI-Fab and 1 mg/l for Fab in Trichoderma in fermenters (Nyyssonen et al., 1993) and such production is very cheap compared to whole antibody production in mammalian cells (hybridoma, myeloma, CHO).

The fragments can be produced as Fab's or as Fv's, but additionally it has been shown that a VH and a VL can be genetically linked in either order by a flexible polypeptide linker, which combination is known as an scFv.

### Isolated Nucleic Acid Molecule/Vector/Host Cell

In one aspect the invention relates to an isolated nucleic acid molecule encoding at least a part of the binding member as defined above. In one embodiment the nucleic acid molecule encodes a light chain and another nucleic acid encodes a heavy chain. The two nucleic acid molecule may be separate or they may be fused into one nucleic acid molecule, optionally spaced apart by a linker sequence. In particular in relation to antibody fragments the nucleic acid molecule may encode the whole binding member; however, dependent on the design of the binding member, this may also be relevant for some larger binding members. The nucleic acid molecule is preferably a DNA sequence, more preferably a DNA sequence comprising in its upstream end regulatory elements promoting the expression of the binding member once the nucleic acid molecule is arranged in a host cell.

Accordingly, in one embodiment the invention relates to a polynucleotide selected from the group consisting of
i) a polynucleotide comprising a sequence selected from the nucleotide sequence of Example 6,
   a polynucleotide encoding a binding member comprising one or more of the amino acid sequence selected from the group of SEQ ID NO 3, 4, 12 or 13,
ii) a polynucleotide encoding a fragment of a polypeptide encoded by polynucleotides i), wherein said fragment
   a) is capable of recognising an antigen also being recognised by the binding member of ii), and/or
   b) is capable of binding selectively to an antigen, wherein said antigen is also bound selectively by the binding member of ii), and/or
   c) has a substantially similar or higher binding affinity to Pneumolysin as a binding domain comprising a predetermined sequence, such as SEQ ID NO 3, 4, 12 or 13,
iii) a polynucleotide, the complementary strand of which hybridizes under stringent conditions, with a polynucleotide as defined in any of i), ii), iii), and encodes a polypeptide as defined in iii),
iv) a polynucleotide comprising a nucleotide sequence which is degenerate to the nucleotide sequence of a polynucleotide as defined in any of i)-iv),
   and the complementary strand of such a polynucleotide.

The invention further relates to a vector comprising the nucleic acid molecule as defined above, either one vector per nucleic acid, or two or more nucleic acids in the same vector. The vector preferably comprises a nucleotide sequence which regulates the expression of the antibody encoded by the nucleic acid molecule.

In yet another aspect the invention relates to a host cell comprising the nucleic acid molecule as defined above.

Also, the invention relates to a cell line engineered to express the binding member as defined above, this cell line for example being a hybridoma of a murine lymphocyte and an immortalised cell line. The cell line may be any suitable cell line, however the cell line P3 is preferred. In another embodiment a CHO cell line is preferred.

### Purification of Binding Members

After production the binding members according to the invention are preferably purified. The method of purification used is dependent upon several factors including the purity required, the source of the antibody, the intended use for the antibody, the species in which the antibody was produced, the class of the antibody and, when the antibody is a monoclonal antibody, the subclass of the antibody.

Any suitable conventional methods of purifying polypeptides comprising antibodies include precipitation and column chromatography and are well known to one of skill in the purification arts, including cross-flow filtration, ammonium sulphate precipitation, affinity column chromatography, gel electrophoresis and the like may be used.

The method of purifying an antibody with an anti-immunoglobulin antibody can be either a single purification procedure or a sequential purification procedure. Methods of single and sequential purification are well known to those in the purification arts. In a single-step purification procedure, the antibody is specifically bound by a single anti-immunoglobulin antibody. Non-specifically bound molecules are removed in a wash step and the specifically bound molecules are specifically eluted. In a sequential purification procedure, the antibody is specifically bound to a first anti-immunoglobulin antibody, non-specifically bound molecules are removed in a wash step, and the specifically bound molecules are specifically eluted. The eluant from the first anti-immunoglobulin antibody is then specifically bound to a second anti-immunoglobulin antibody. The non-specifically bound molecules are removed in a wash step, and the specifically bound molecules are specifically eluted. In a preferred embodiment, the antibody is sequentially purified by a first and second anti-immunoglobulin antibody selected from the group consisting of antibodies which specifically bind heavy and light chain constant regions.

A commonly used method of purification is affinity chromatography in which the antibody to be purified is bound by protein A, protein G or by an anti-immunoglobulin antibody. Another method of affinity chromatography, which is well known to those of skill in the art, is the specific binding of the antibody to its respective antigen.

In particular for purifying a multispecific, including a bispecific antibody, a sequential purification procedure may be used, wherein the bispecific antibody comprising two or more variable domains is specifically bound to a first antigen and then to a second antigen.

In an alternative embodiment, a bispecific antibody comprising two or more variable regions is purified by sequential purification by specifically binding the antibody to a first antigen in a first purification step and to a second antigen in a second purification step.

The method of purifying an antibody with an anti-immunoglobulin antibody can be either a single purification procedure or a sequential purification procedure. Methods of single and sequential purification are well known to those in the purification arts. In a single-step purification procedure, the antibody is specifically bound by a single anti-immunoglobulin antibody. Non-specifically bound molecules are removed in a wash step and the specifically bound molecules are specifically eluted. In a sequential purification procedure, the antibody is specifically bound to a first anti-immunoglobulin antibody, non-specifically bound molecules are removed in a wash step, and the specifically bound molecules are specifically eluted. The eluant from the first anti-immunoglobulin antibody is then specifically bound to a second anti-immunoglobulin antibody. The non-specifically bound molecules are removed in a wash step, and the specifically bound molecules are specifically eluted. In a preferred embodiment, the antibody is sequentially purified by a first and second anti-immunoglobulin antibody selected from the group consisting of antibodies which specifically bind heavy and light chain constant regions. In a more preferred embodiment, the antibody is sequentially purified by a first and second anti-immunoglobulin antibody selected from the group consisting of antibodies which specifically bind the heavy chain constant region of IgG and light chain constant regions of kappa and lambda. In an even more preferred embodiment, the anti-immunoglobulin antibody is selected from the group consisting of antibodies which specifically bind the light chain constant regions of kappa and lambda.

### Diagnostic Methods

The present invention also describes a diagnostic system, preferably in kit form, for assaying for the presence of Streptococcus, in particular Streptococcus pneumoniae, in a biological sample where it is desirable to detect the presence, and preferably the amount, of bacteria in a sample according to the diagnostic methods described herein.

The diagnostic system includes, in an amount sufficient to perform at least one assay, a binding member composition according to the present invention, preferably as a separately packaged reagent, and more preferably also instruction for use.

The biological sample can be a tissue, tissue extract, fluid sample or body fluid sample, such as blood, plasma or serum.

Packaged refers to the use of a solid matrix or material such as glass, plastic (e.g., polyethylene, polypropylene or polycarbonate), paper, foil and the like capable of holding within fixed limits a binding member of the present invention. Thus, for example, a package can be a glass vial used to contain milligram quantities of a contemplated labelled binding member preparation, or it can be a microtiter plate well to which microgram quantities of a contemplated binding member has been operatively affixed, i.e., linked so as to be capable of binding a ligand.

“Instructions for use” typically include a tangible expression describing the reagent concentration or at least one assay method parameter such as the relative amounts of reagent and sample to be admixed, maintenance time periods for reagent/sample admixtures, temperature, buffer conditions and the like.

A diagnostic system of the present invention preferably also includes a label or indicating means capable of signalling the formation of a binding reaction complex containing a binding member complexed with the preselected ligand.

Any label or indicating means can be linked to or incorporated in an expressed polypeptide, or phage particle that is used in a diagnostic method. Such labels are themselves well-known in clinical diagnostic chemistry.

The labeling means can be a fluorescent labeling agent that chemically binds to antibodies or antigens without denaturing them to form a fluorochrome (dye) that is a useful immunofluorescent tracer. Suitable fluorescent labeling agents are fluorochromes such as fluorescein isocyanate (FIC), fluorescein isothiocyante (FITC), 5-dimethylamine-1-naphthalenesulfonyl chloride (DANSC), tetramethylrhodamine isothiocyanate (TRITC), lissamine, rhodamine 8200 sulphonyl chloride (RB 200 SC) and the like. A description of immunofluorescence analysis techniques is found in DeLuca, “Immunofluorescence Analysis”, in Antibody As a Tool, Marchalonis, et al., eds., John Wiley & Sons, Ltd., pp. 189-231 (1982), which is incorporated herein by reference.

In preferred embodiments, the indicating group is an enzyme, such as horseradish peroxidase (HRP), glucose oxidase, or the like. In such cases where the principal indicating group is an enzyme such as HRP or glucose oxidase, additional reagents are required to visualize the fact that a receptor-ligand complex (immunoreactant) has formed. Such additional reagents for HRP include hydrogen peroxide and an oxidation dye precursor such as diaminobenzidine. An additional reagent useful with glucose oxidase is 2,2′-amino-di-(3-ethyl-benzthiazoline-G-sulfonic acid) (ABTS).

Radioactive elements are also useful labeling agents and are used illustratively herein. An exemplary radiolabeling agent is a radioactive element that produces gamma ray emissions. Elements which themselves emit gamma rays, such as ¹²⁴I, ¹²⁵I, ¹²⁸I, ¹³²I and ⁵¹Cr represent one class of gamma ray emission-producing radioactive element indicating groups. Particularly preferred is ¹²⁵I. Another group of useful labeling means are those elements such as ¹¹C, ¹⁸F, ¹⁵O and ¹³N which themselves emit positrons. The positrons so emitted produce gamma rays upon encounters with electrons present in the animal's body. Also useful is a beta emitter, such as ¹¹¹ indium or ³H.

The linking of labels, i.e., labeling of, polypeptides and proteins or phage is well known in the art. For instance, proteins can be labelled by metabolic incorporation of radioisotope-containing amino acids provided as a component in the culture medium. See, for example, Galfre et al., Meth. Enzymol., 73:3-46 (1981). The techniques of protein conjugation or coupling through activated functional groups are particularly applicable. See, for example, Aurameas, et al., Scand. J. Immunol., Vol. 8 Suppl. 7:7-23 (1978), Rodwell et al., Biotech., 3:889-894 (1984), and U.S. Pat. No. 4,493,795.

The diagnostic systems can also include a specific binding agent, preferably as a separate package. A “specific binding agent” is a molecular entity capable of selectively binding a binding member species of the present invention or a complex containing such a species, but is not itself a binding member of the present invention. Exemplary specific binding agents are antibody molecules, complement proteins or fragments thereof, S. aureus protein A, and the like. Preferably the specific binding agent binds the binding member species when that species is present as part of a complex.

In preferred embodiments, the specific binding agent is labelled. However, when the diagnostic system includes a specific binding agent that is not labelled, the agent is typically used as an amplifying means or reagent. In these embodiments, the labelled specific binding agent is capable of specifically binding the amplifying means when the amplifying means is bound to a reagent species-containing complex.

The diagnostic kits of the present invention can be used in an “ELISA” format to detect the quantity of a preselected ligand in a fluid sample. “ELISA” refers to an enzyme-linked immunosorbent assay that employs an antibody or antigen bound to a solid phase and an enzyme-antigen or enzyme-antibody conjugate to detect and quantify the amount of an antigen present in a sample and is readily applicable to the present methods.

Thus, in some embodiments, a binding member of the present invention can be affixed to a solid matrix to form a solid support that comprises a package in the subject diagnostic systems.

A reagent is typically affixed to a solid matrix by adsorption from an aqueous medium although other modes of affixation applicable to proteins and polypeptides can be used that are well known to those skilled in the art. Exemplary adsorption methods are described herein.

Useful solid matrices are also well known in the art. Such materials are water insoluble and include the cross-linked dextran available under the trademark SEPHADEX from Pharmacia Fine Chemicals (Piscataway, N.J.); agarose; beads of polystyrene beads about 1 micron to about 5 millimeters in diameter available from Abbott Laboratories of North Chicago, Ill.; polyvinyl chloride, polystyrene, cross-linked polyacrylamide, nitrocellulose- or nylon-based webs such as sheets, strips or paddles; or tubes, plates or the wells of a microtiter plate such as those made from polystyrene or polyvinylchloride.

The binding member species, labelled specific binding agent or amplifying reagent of any diagnostic system described herein can be provided in solution, as a liquid dispersion or as a substantially dry power, e.g., in lyophilized form. Where the indicating means is an enzyme, the enzyme's substrate can also be provided in a separate package of a system. A solid support such as the before-described microtiter plate and one or more buffers can also be included as separately packaged elements in this diagnostic assay system.

### Diagnostic Methods

The present invention also contemplates various assay methods for determining the presence, and preferably amount, of a Streptococcus, in particular Streptococcus pneumoniae, typically present in a biological sample.

Accordingly, the present invention relates to a method of detecting or diagnosing a disease or disorder associated with Pneumococcus in an individual comprising
providing a biological sample from said individual
adding at least one binding member as defined above to said biological sample,
detecting binding members bound to said biological sample, thereby detecting or diagnosing the disease or disorder.

The bound binding members may be detected either directly or indirectly, to the amount of the Streptococcus in the sample.

Those skilled in the art will understand that there are numerous well known clinical diagnostic chemistry procedures in which a binding reagent of this invention can be used to form an binding reaction product whose amount relates to the amount of the ligand in a sample. Thus, while exemplary assay methods are described herein, the invention is not so limited.

Various heterogenous and homogeneous protocols, either competitive or noncompetitive, can be employed in performing an assay method of this invention.

Binding conditions are those that maintain the ligand-binding activity of the receptor. Those conditions include a temperature range of about 4 to 50 degrees Centigrade, a pH value range of about 5 to 9 and an ionic strength varying from about that of distilled water to that of about one molar sodium chloride.

The detecting step can be directed, as is well known in the immunological arts, to either the complex or the binding reagent (the receptor component of the complex). Thus, a secondary binding reagent such as an antibody specific for the receptor may be utilized.

Alternatively, the complex may be detectable by virtue of having used a labelled receptor molecule, thereby making the complex labelled. Detection in this case comprises detecting the label present in the complex.

A further diagnostic method may utilize the multivalency of a binding member composition of one embodiment of this invention to cross-link ligand, thereby forming an aggregation of multiple ligands and polypeptides, producing a precipitable aggregate. This embodiment is comparable to the well-known methods of immune precipitation. This embodiment comprises the steps of admixing a sample with a binding member composition of this invention to form a binding admixture under binding conditions, followed by a separation step to isolate the formed binding complexes. Typically, isolation is accomplished by centrifugation or filtration to remove the aggregate from the admixture. The presence of binding complexes indicates the presence of the preselected ligand to be detected.

### Pharmaceutical Compositions

In a preferred aspect the present invention contemplates pharmaceutical compositions useful for practising the therapeutic methods described herein. Pharmaceutical compositions of the present invention contain a physiologically tolerable carrier together with at least one species of binding member as described herein, dissolved or dispersed therein as an active ingredient. In a preferred embodiment, the pharmaceutical composition is not immunogenic when administered to a human individual for therapeutic purposes, unless that purpose is to induce an immune response.

In one aspect the invention relates to a pharmaceutical composition comprising at least one binding member as defined above. In a preferred embodiment the pharmaceutical composition comprises at least two different binding members as defined above in order to increase the effect of the treatment.

As used herein, the terms “pharmaceutically acceptable”, “physiologically tolerable” and grammatical variations thereof, as they refer to compositions, carriers, diluents and reagents, are used interchangeably and represent that the materials are capable of administration to or upon a human without the production of undesirable physiological effects such as nausea, dizziness, gastric upset and the like.

The preparation of a pharmacological composition that contains active ingredients dissolved or dispersed therein is well understood in the art. Typically such compositions are prepared as sterile injectables either as liquid solutions or suspensions, aqueous or non-aqueous; however, solid forms suitable for solution, or suspensions, in liquid prior to use can also be prepared. The preparation can also be emulsified.

The active ingredient can be mixed with excipients which are pharmaceutically acceptable and compatible with the active ingredient and in amounts suitable for use in the therapeutic methods described herein. Suitable excipients are, for example, water, saline, dextrose, glycerol, ethanol or the like and combinations thereof. In addition, if desired, the composition can contain minor amounts of auxiliary substances such as wetting or emulsifying agents, pH buffering agents and the like, which enhance the effectiveness of the active ingredient.

The pharmaceutical composition of the present invention can include pharmaceutically acceptable salts of the components therein. Pharmaceutically acceptable salts include the acid addition salts (formed with the free amino groups of the polypeptide) that are formed with inorganic acids such as, for example, hydrochloric or phosphoric acids, or such organic acids as acetic, tartaric, mandelic and the like. Salts formed with the free carboxyl groups can also be derived from inorganic bases such as, for example, sodium, potassium, ammonium, calcium or ferric hydroxides, and such organic bases as isopropylamine, trimethylamine, 2-ethylamino ethanol, histidine, procaine and the like.

Physiologically tolerable carriers are well known in the art. Exemplary of liquid carriers are sterile aqueous solutions that contain no materials in addition to the active ingredients and water, or contain a buffer such as sodium phosphate at physiological pH value, physiological saline or both, such as phosphate-buffered saline. Still further, aqueous carriers can contain more than one buffer salt, as well as salts such as sodium and potassium chlorides, dextrose, propylene glycol, polyethylene glycol and other solutes.

Liquid compositions can also contain liquid phases in addition to and to the exclusion of water. Exemplary of such additional liquid phases are glycerin, vegetable oils such as cottonseed oil, organic esters such as ethyl oleate, and water-oil emulsions.

A pharmaceutical composition contains a binding member of the present invention, typically an amount of at least 0.1 weight percent of antibody per weight of total pharmaceutical composition. A weight percent is a ratio by weight of antibody to total composition. Thus, for example, 0.1 weight percent is 0.1 grams of antibody per 100 grams of total composition.

The invention also relates to a method for preparing a medicament or pharmaceutical composition comprising an antibody of the invention, the medicament being used for immunotherapy of a disease or disorder associated with Streptococcus, in particular Streptococcus pneumoniae, such as pneumonia, meningitis and sepsis, comprising admixing at least one binding member as defined above with a physiologically acceptable carrier.

Furthermore, the invention relates to the use of a binding member as defined above for the production of a pharmaceutical composition for the treatment of a disease or disorder associated with Streptococcus, in particular Streptococcus pneumoniae, such as pneumonia, meningitis and sepsis.

The pharmaceutical composition may also be a kit-in-part further including an antibiotic agent, such as antibiotics selected from -lactams, cephalosporins, penicilins and aminoglycosides, and/or include an immunostimulating agent, such as cytokines, interferons, growth factors, for example GCSF or GM-CSF. The kit-in-part may be used for simultaneous, sequential or separate administration.

Furthermore, the pharmaceutical composition may include the binding member according to the invention in combination with the Streptococcus protein Pneumolysin, in particular as a vaccine. It has been found that by combining the binding member according to the invention with the protein Pneumolysin, the immunising properties of the combination product is better than for the protein Pneumolysin alone. This may be due to the fact that the protein Pneumolysin is presented to the immune system by the binding member.

In another embodiment, the antibody according to the invention is combined with another antibody against Streptococcus pneumoniae, such as another anti-Pneumolysin antibody, for example a non-haemolytic anti-Pneumolysin antibody.

The antibody according to the invention may also be an anti-PsaA antibody as described in International patent application no. PCT/DK2004/000492.

### Therapeutic Methods

The binding members according to the present invention are particular useful in therapeutic methods due to their high affinity and specificity. Accordingly, the binding members can be used immunotherapeutically towards a disease or disorder associated with Streptococcus, in particular Streptococcus pneumoniae, such as pneumonia, meningitis and sepsis.

The term “immunotherapeutically” or “immunotherapy” as used herein in conjunction with the binding members of the invention denotes both prophylactic as well as therapeutic administration. Thus, the binding members can be administered to highrisk patients in order to lessen the likelihood and/or severity of disease, administered to patients already evidencing active infection, or administered to patients at risk of infection.

The dosage ranges for the administration of the binding members of the invention are those large enough to produce the desired effect in which the symptoms of the disease are ameliorated or the likelihood of infection decreased. Generally, the dosage will vary with the age, condition, sex and extent of the disease in the patient and can be determined by one of skill in the art. The dosage can be adjusted by the individual physician in the event of any complication.

A therapeutically effective amount of an binding member of this invention is typically an amount of antibody such that when administered in a physiologically tolerable composition is sufficient to achieve a plasma concentration of from about 0.1 microgram (μg) per milliliter (ml) to about 100 μg/ml, preferably from about 1 μg/ml to about 5 μg/ml, and usually about 5 μg/ml. Stated differently, the dosage can vary from about 0.1 mg/kg to about 300 mg/kg, preferably from about 0.2 mg/kg to about 200 mg/kg, most preferably from about 0.5 mg/kg to about 20 mg/kg, in one or more dose administrations daily, for one or several days.

The binding members of the invention can be administered parenterally by injection or by gradual infusion over time. Although the infection may be systemic and therefore most often treated by intravenous administration of pharmaceutical compositions, other tissues and delivery means are contemplated where there is a likelihood that targeting a tissue will result in a lessening of the disease. Thus, antibodies of the invention can be administered parenterally, such as intravenously, intraperitoneally, intramuscularly, subcutaneously, intracavity, transdermally, and can be delivered by peristaltic means.

The pharmaceutical compositions containing a binding member of this invention are conventionally administered intravenously, as by injection of a unit dose, for example. The term “unit dose” when used in reference to a pharmaceutical composition of the present invention refers to physically discrete units suitable as unitary dosage for the subject, each unit containing a predetermined quantity of active material calculated to produce the desired therapeutic effect in association with the required diluent; i.e., carrier, or vehicle.

The therapeutic method may further include the use of a kit-in-part as defined above.

### Passive Immune Protection

The binding members may be particular useful for passive immune protection, whereby the binding member neutralise the action of Pneumolysin. The binding member may be evaluated in an assay as described in Example 1. The result of the assay demonstrates that administration of a binding member towards Pneumolysin may prolong survival upon S. pneumoniae infection in mice and thus induction of passive immune protection.

### Active Immune Protection

The antigenic epitopes of the invention can be used as vaccines to stimulate an immunological response in a mammal directed against Pneumolysin, a mammal for example being a mouse, dog, cat, swine, horse, bovine etc. and preferably a human being. Such an response may include induction of Pneumolysin specific antibodies. Antibodies directed against the antigenic epitopes of the invention can inhibit Pneumolysin function as described above, and immunisation may further be used for prophylactic treatment and infection caused by S. pneumoniae.

### Pneumolysin Peptide

In an aspect the invention relates to a Pneumolysin peptide comprising an epitope recognised by a binding member according to the invention. Preferably the Pneumolysin peptide, fragment or variants preferably comprise an amino acid sequence identified by SEQ ID NO 27, 28, 29, 30, 31, 32, 33, 34, 35 or 36. A Pneumolysin peptide according to the invention may be a peptide consisting of amino acid 1-436 of SEQ ID NO 11. Further included are fragments and variants of the Pneumolysin peptide consisting of amino acid 1-436 of SEQ ID NO 11, this includes fragments comprising amino acid 50-436, or more preferably amino acid 100-436 of Pneumolysin as identified by SEQ ID NO 11. In specific embodiments the Pnemolysin peptide comprise amino acid 200-436 or 300-436 of Pneumolysin as identified by SEQ ID NO 11. Variants or homologues of Pneumolysin peptides may be defined as homologues in relation to binding members as described above.

The Pneumolysin peptide, fragment or variants preferably comprise an amino acid sequence identified by SEQ ID NO 27, 28, 29, 30, 31, 32, 33, 34, 35 or 36. It is preferred that the Pneumolysin peptide is constituted by at the most 100, such as 80, 60, 40, 30, 25, 20, 15 or such as 12 amino acids. It may further be preferred that the Pneumolysin peptide is constituted by at the least 12, such as 15, 20, 25, 30, 40, 60, 80, or such as at least 100 amino acids.

In specific embodiments the Pneumolysin peptide fragment s are identified by SEQ ID NO 27, 29, 30, 31 or 32.

The Pneumolysin peptides may be used as antigenic epitopes capable of stimulating the immune system.

### Vaccine Composition

A vaccine composition according to the invention can be formulated according to known methods such as by the admixture of one or more pharmaceutically acceptable excipients or carriers with the active agent, preferably acceptable for administration to humans. Examples of such excipients, carriers and methods of formulation may be found e.g. in Remington's Pharmaceutical Sciences (Maack Publishing Co, Easton, Pa.). To formulate a pharmaceutically acceptable composition suitable for effective administration, such compositions will according to the invention contain an effective amount of a Pneumolysin polypeptide or an analog there of.

Vaccine compositions according to the invention may be administered to an individual in therapeutically effective amounts. The effective amount may vary according to a variety of factors such as the individual's condition, weight, sex and age. Other factors include the mode of administration.

In the following vaccine compositions are meant to encompass compositions useful for therapeutic use, including stimulating an immune response.

To obtain vaccines or immunogenic compositions it may be required to combine the Pneumolysin peptide or analog molecules with various materials such as adjuvants, immunostimulatory components and/or carriers. Adjuvants are included in the vaccine composition to enhance the specific immune response.

Such adjuvants may be any compound comprising an adjuvant effect known to the person skilled in the art. For example such adjuvants could be of mineral, bacterial, plant, synthetic or host origin or they could be oil in water emulsions.

Adjuvants could be selected from the group consisting of: AlK(SO₄)₂, AlNa(SO₄)₂, AlNH₄ (SO₄), silica, alum, Al(OH)₃, Ca₃ (PO₄)₂, kaolin, carbon, aluminum hydroxide, muramyl dipeptides, N-acetyl-muramyl-L-threonyl-D-isoglutamine (thr-DMP), N-acetyl-nornuramyl-L-alanyl-D-isoglutamine (CGP 11687, also referred to as nor-MDP), N-acetylmuramyul-L-alanyl-D-isoglutaminyl-L-alanine-2-(1′2′-dipalmitoyl-sn glycero-3-hydroxphosphoryloxy)-ethylamine (CGP 19835A, also referred to as MTP-PE), RIBI (MPL+TDM+CWS) in a 2% squalene/Tween-80™. emulsion, lipopolysaccharides and its various derivatives, including lipid A, Freund's Complete Adjuvant (FCA), Freund's Incomplete Adjuvants, Merck Adjuvant 65, polynucleotides (for example, poly IC and poly AU acids), wax D from Mycobacterium, tuberculosis, substances found in Corynebacterium parvum, Bordetella pertussis, and members of the genus Brucella, liposomes or other lipid emulsions, Titermax, ISCOMS, Quil A, ALUN (see U.S. Pat. No. 58,767 and U.S. Pat. No. 5,554,372), Lipid A derivatives, choleratoxin derivatives, HSP derivatives, LPS derivatives, synthetic peptide matrixes or GMDP, Interleukin 1 and Interleukin 2.

A large number of adjuvants have been described and used for the generation of antibodies in laboratory animals, such as mouse, rats and rabbits. In such setting the tolerance of side effect is rather high as the main aim is to obtain a strong anti-body response.

For use and for approval for use in pharmaceuticals, and especially for use in humans it is required that the components of the vaccine composition, including the adjuvant, are well characterised. It is further required that the composition has minimal risk of any adverse reaction, such as granuloma, abscesses or fever.

In a preferred embodiment the vaccine composition is suitable for administration to a human subject, thus a preferred adjuvant are suitable for administration to a human subject.

Adjuvants useful in therapeutic vaccines may be mineral salts, such as aluminium hydroxide and aluminium or calcium phosphates gels, oil emulsions and surfactant based formulations such as MF59 (microfluidised detergent stabilised oil in water emulsion), QS21 (purified saponin), AS02 (SBAS2, oil-in-water emulsion+monophosphoryl lipid A (MPL)+QS21), Montanide ISA 51 and ISA-720 (stabilised water in oil emulsion), Adjuvant 65 (containing peanut oil, mannide monooleate and aluminum monostearate), RIBI ImmunoChem Research Inc., Hamilton, Utah), particulate adjuvants, such as virosomes (unilamellar liposomal cehicles incorporating influenza haemagglutinin), AS04 (Al salt with MPL), ISCOMS (structured complex of saponins and lipids (such as cholesterol), polyactide co-glycolide (PLG), microbial derivatives (natural and synthetic) such as monophosphoryl lipid A (MPL), Detox (MPL+M. Phlei cell wall skeleton), AGP (RC-529 (synthetic acylated monosaccharide)), DC_chol (lipoidal immunostimulators able to self organise into liposomes), OM-174 (lipid A derivative), CpG motifs (synthetic oligonucleotides containing immunostimulatory CpG motifs), modified bacterial toxins, LT and CT, with non-toxic adjuvant effects, Endogenous human immunomodulators, e.g., hGM-CSF or hlL-12 or Immudaptin (C3d tandem array), inert vehicles such as gold particles.

In some embodiments, the vaccine composition may further comprise one or more additional immunostimulatory components. These include, without limitation, muramyldipeptide (MDP); e.g. N-acetyl-muramyl-L-alanyl-D-isoglutamine (ala-MDP), Nacetyl-muramyl-L-threonyl-D-isoglutamine (thr-MDP), N-acetyl-nor-muramyl-Lalanyl-D-isoglutamine (CGP 11637, nor-MDP) and N-acetyl-muramyl-L-alanyl-Disoglutaminyl-L-alanine-2-(1′-2′-dipalmitoyl-sn-glycero-3-hydroxyphosphoryloxy)ethylamine (CGP 19835A, MTP-PE), dimethylglycine, tuftsin, and trehalose dimycolate. monophosphoryl-lipid A (MPL), and formyl-methionine containing tri-peptides such as N-formyl-Met-Leu-Phe. Such compounds are commercially available from Sigma Chemical Co. (St. Louis, Mo.) and RIBI ImmunoChem Research, Inc. (Hamilton, Mont.), for example.

A carrier may be present independently of an adjuvant. The function of a carrier can for example be to increase the molecular weight of in particular survivin fragments in order to increase their activity or immunogenicity, to confer stability, to increase the biological activity, or to increase serum half-life. The carrier may be any suitable carrier known to the person skilled in the art. A carrier protein could be but is not limited to keyhole limpet haemocyanin, serum proteins such as transferrin, bovine serum albumin, human serum albumin, thyroglobulin or ovalbumin, immunoglobulins, or hormones, such as insulin or palmitic acid. For immunization of humans, the carrier must be a physiologically acceptable carrier acceptable to humans and safe. However, tetanus toxoid and/or diptheria toxoid are suitable carriers in one embodiment of the invention. Alternatively, the carrier may be dextrans for example sepharose.

In an embodiment the vaccine composition comprise a Pneumolysin peptide comprising an amino acid sequence identified by SEQ ID NO 27, 28, 29, 30, 31 or 32. Vaccines comprising peptides comprising an amino acid sequence identified by SEQ ID NO 29, 30 or 31 are preferred. Especially preferred are peptides comprising the amino acid sequence of 400-436, 422-436 or 425-436 of pneumolysin as identified by SEQ ID NO 11.

It is preferred that the Pneumolysin peptide is constituted by at the most 100, such as 80, 60, 40, 20, 15, 12, 10 8 or such as 6 amino acids. It may further be preferred that the Pneumolysin peptide is constituted by at the least 6, such as 8, 10, 12, 15, 20, 25, 30, 40, 60, 80, or such as at least 100 amino acids. In an embodiment the vaccine composition comprise at least one Pneumolysin peptide identified by SEQ ID NO 27, 28, 27, 30, 31, 32, 33, 34, 35 or 36. Vaccines comprising peptides identified by SEQ ID NO 28, 29, 30 or 31 are preferred. Especially preferred are peptides comprising the amino acid sequences identified as AA 423-438, 424-437, 425-436 or 426-436 of pneumolysin as identified by SEQ ID NO 11.

A vaccine composition capable of stimulating an immune response is preferred. It is particularly relevant that the vaccine composition is capable of inducing an antibody response upon administration. Mostly preferred are vaccines capable of inducing a Pneumolysin inhibiting response, by inducing the production of antibodies capable of inhibition the lytic activities of Pneumolysin. Other preferred embodiments include antibodies capable of enhancing phagocytosis of Pneumolysin. Such antibodies may be characterised by comprising a variable region as the binding member described here in.

### DETAILED DESCRIPTION OF DRAWINGS

FIG. 1. Schematic drawing of a Fab fragment.

The antigen pocket composed of VL, CDR1, CDR2, CDR3 and VH, CDR1, CDR2, CDR3 is shown.

FIG. 2. Pneumolysin amino acid sequence having SEQ ID NO 11.

The amino acid sequence of Pneumolysin corresponding to the sequence of Genebank no. X52474 is shown.

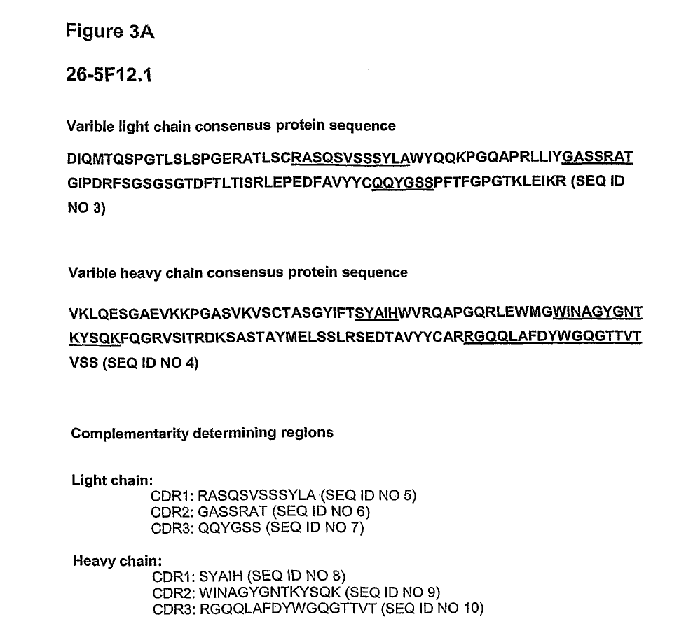

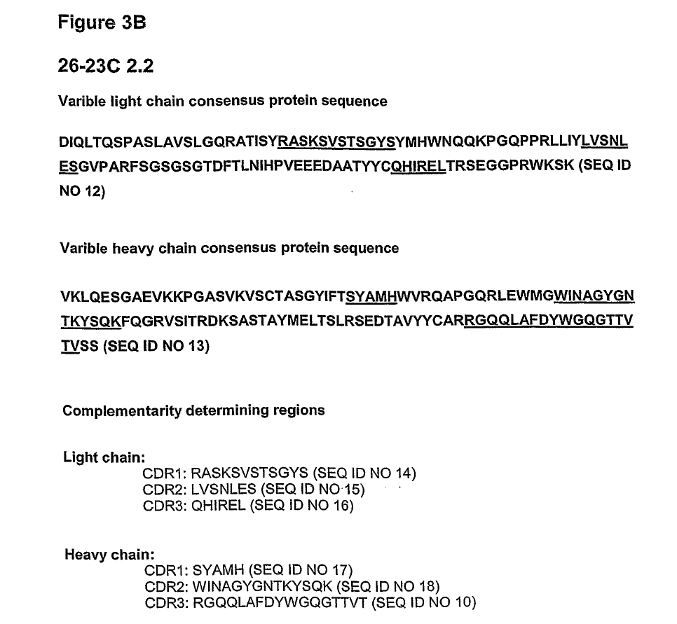

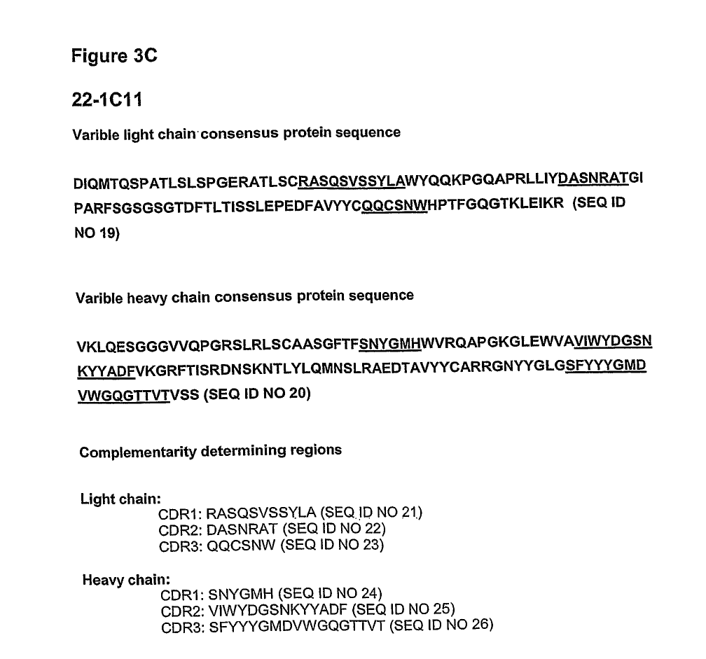

FIG. 3. Anti-Pneumolysin light chain and heavy chain variable segments.

FIG. 3A includes the consensus sequences of the variable light and heavy chain and the complementarity determining regions of antibody 26-5F12.1. FIG. 3B includes the consensus sequences of the variable light and heavy chain and the complementarity determining regions of antibody 26-23C 2.2. FIG. 3C includes the consensus sequences of the variable light and heavy chain and the complementarity determining regions of antibody 22-1C11. The sequences are obtained as described in example 6.

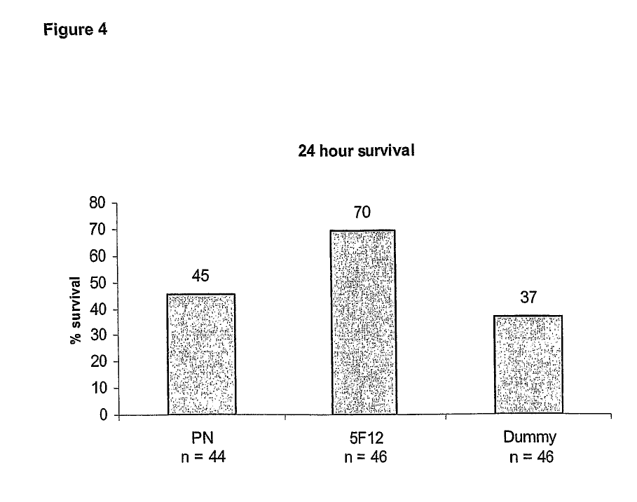

FIG. 4. Survival diagram for mice inoculated with Pneumococcus and antibody. The survival of mice injected with Pneumococcus D39 alone or in combination with penicillin and/or Pneumolysin antibody (26-5F12) evaluated 24 hours after inoculation as described in example 1.

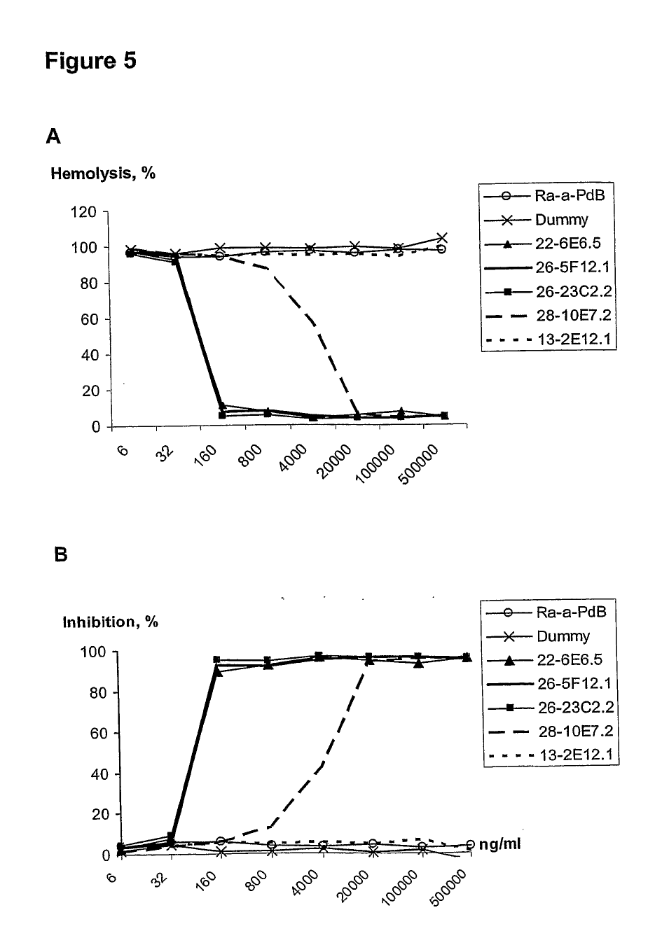

FIG. 5. Antihaemolytic activity of Pneumolysin antibodies.

The anti-haemolytic activity of Pneumolysin antibodies analysed by evaluating the inhibitory effect on Pneumolysin mediated lysis of erythrocytes as described in example 3. Three antibodies (26-5F12, 26-23C 2 and 22-6E6) are particular effective.

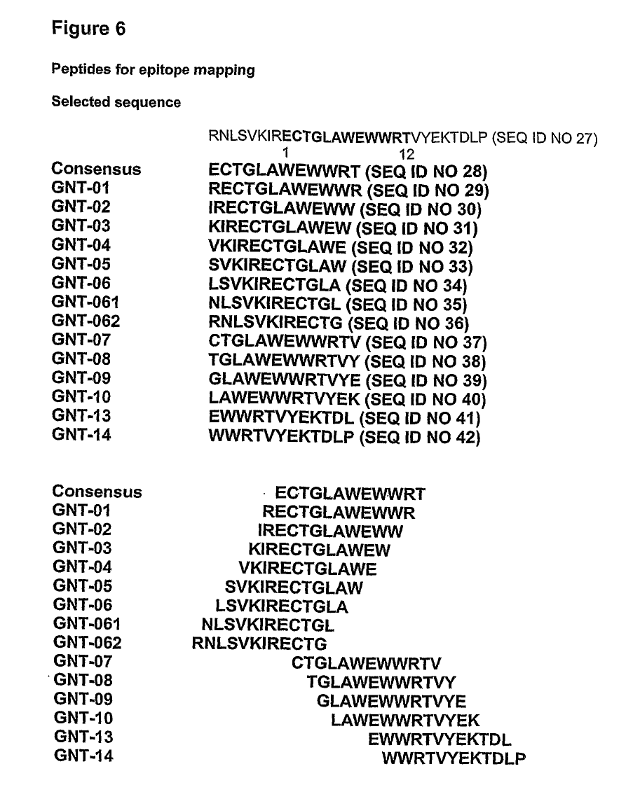

FIG. 6. Peptides for epitope mapping.

An overview of the amino acid sequence 419-446 of Pneumnolysin and various peptide sequences for epitope mapping.

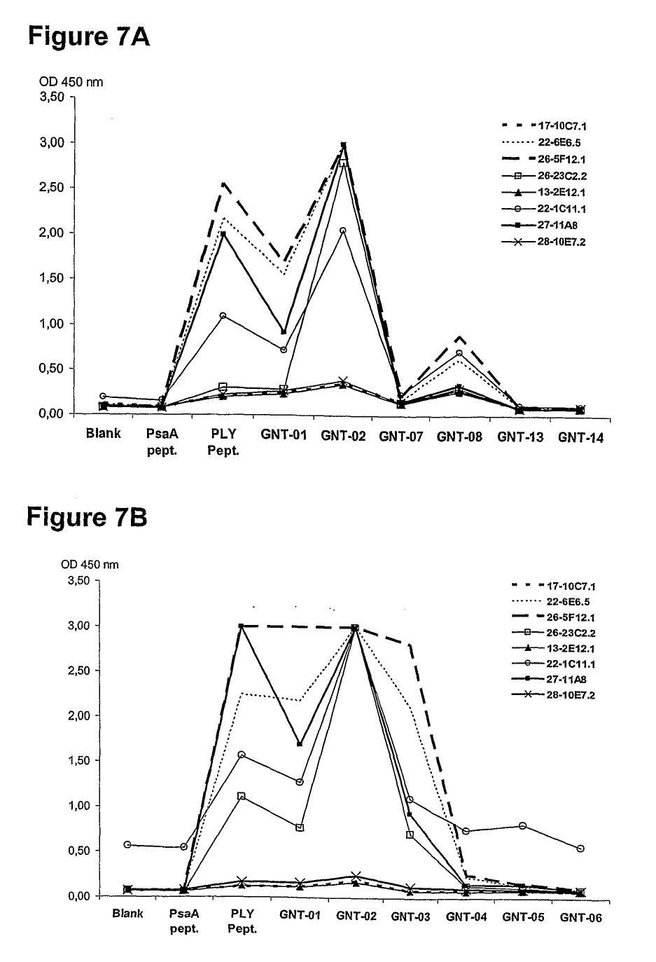

FIG. 7 Pneumolysin antibody epitopes.

FIG. 7A and FIG. 7B are graphic illustrations of the results obtained as described in example 7 related to identification of the antibody epitope.

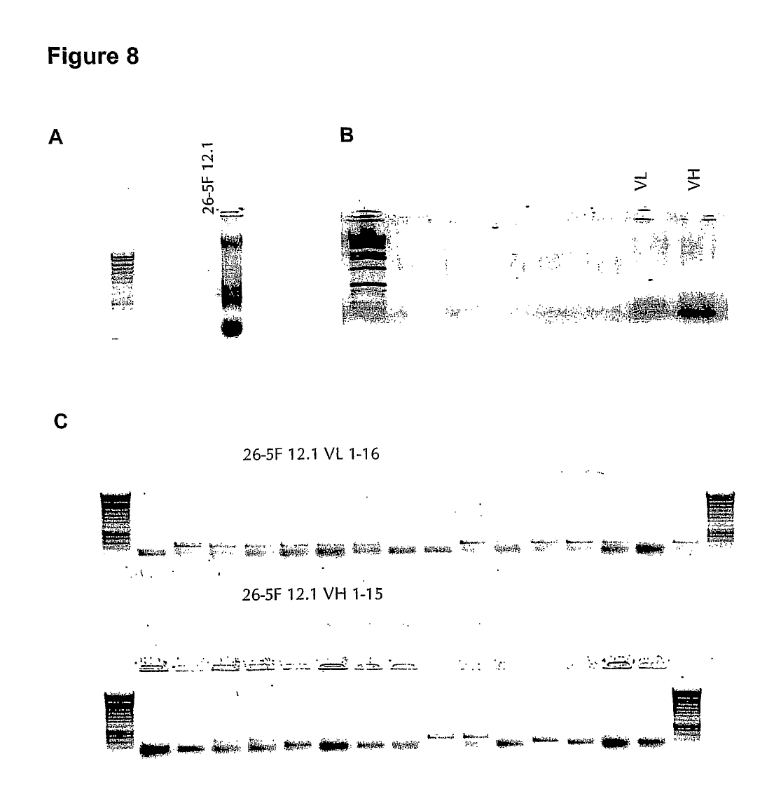

FIG. 8 Isolation of 26-5F12 clones

FIG. 8A shows the total RNA isolated from the 26-5F12 hybridoma cells. The RNA was used for cDNA synthesis of heavy chain and light chain variable regions. The PCR products are shown in FIG. 8B. After cloning the positive transformants were identified using colony PCR (FIG. 8C).

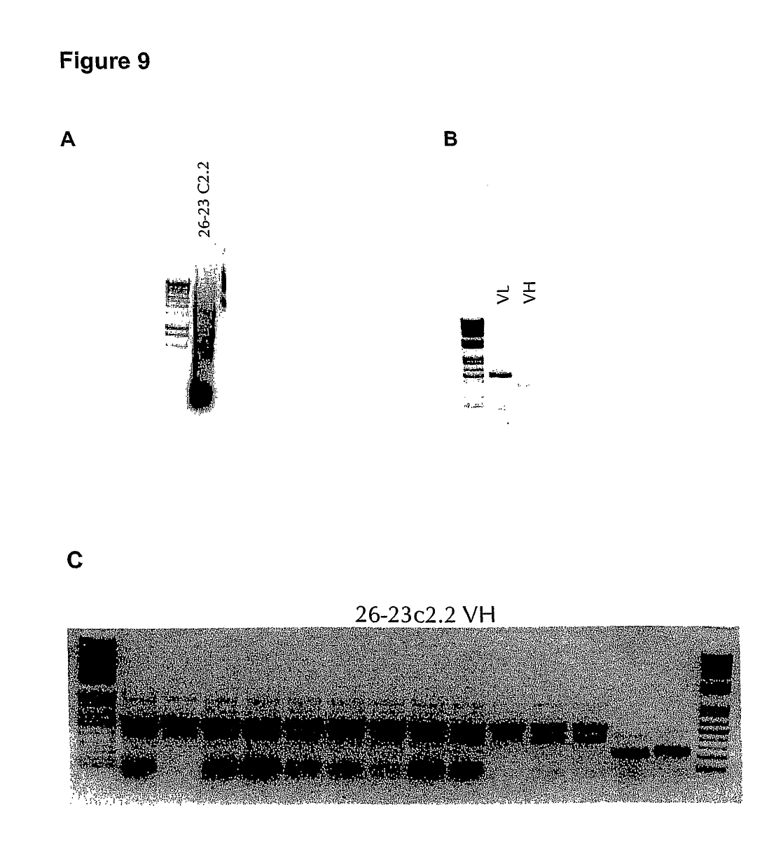

FIG. 9 Isolation of 26-23C2 clones

FIG. 9A shows the total RNA isolated from the 26-23 C2 hybridoma cells. The RNA was used for cDNA synthesis of heavy chain and light chain variable regions. The PCR products are shown in FIG. 9B. After cloning the positive transformants were identified using colony PCR (FIG. 9C).

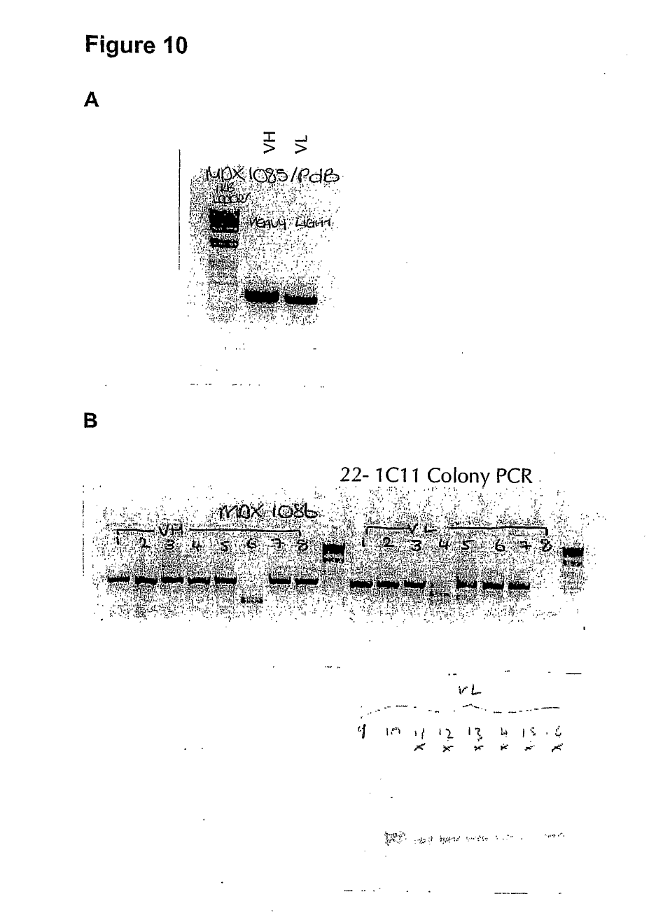

FIG. 10 Isolation of 22 1C11 clones

Total RNA isolated from 22 1C11 hybridoma cells was used for cDNA synthesis of heavy chain and light chain variable regions. The PCR products are shown in FIG. 10BA. After cloning the positive transformants were identified using colony PCR (FIG. 10B).

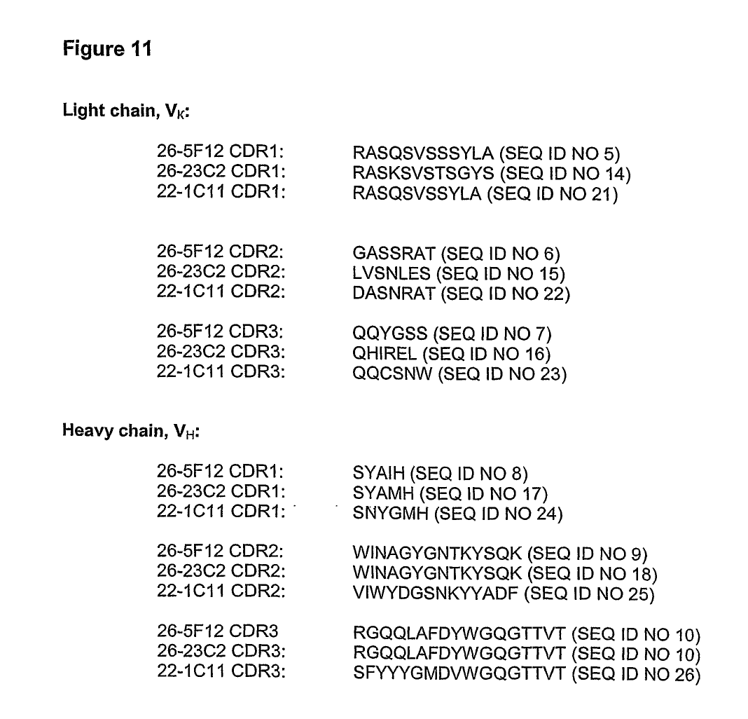

FIG. 11 CDR sequences of 26-5F12, 26-23C2 and 22 1C11.

The sequences of the light and heavy chain CDR's of 26-5F12, 26-23 C2 and 22 1C11 are aligned. The heavy chain of 26-5F12 and 26-23C2 is almost identical whereas CDR 2 and CDR3 of 22 1C11 heavy chain diverge from the sequence of the 6-5F12 and 26-23C2.

### EXAMPLES

The invention is further explained through the examples below; the examples are not to be construed as limiting to the invention.

### Example 1

Study of the effect of antibodies and penicillin on survival of transgenic female mice inoculated with Pneumococcus D39 (type 2)

### Materials

82 transgenic female mice (M-B project no. #249, project name CD64, about 8-12 weeks old)
0.9% saline (AAS)
PBS pH 7.4
Syringes
Needles
5% blood plates
Filtered bovine broth
Solvent ad penicillin
Penicillin 1 million IU (Løven D6726), 10 mg/mouse ˜40 mg/ml
   Strains: Pneumococcus D39 (type 2) (F1/S1/E2)

### Antibodies:

PdB26-5F12.1, 1.0 mg/ml 040520
OmpA6-4B6.1, 1.38 mg/ml

### Method:

Hours −24: The Pneumococcus strain is seeded onto 3×5% blood plate and incubated overnight at 35° C./CO₂.

Hours 0: The Pneumococcus strain is slurried in filtered broth to 108 CFU/ml (cf. MU/F074-01) and diluted to 2×10⁵ CFU/ml (120 μl 10⁸ CFU/ml in 59.88 ml of PBS).

The antibody is diluted to 200 μg/ml:

### 3.00 ml of PdB26-5F12.1+12.00 ml of PBS

### 2.17 ml of OmpA6-4B6.1+12.83 ml of PBS

The mice are treated with bacteria (0.5 ml i.p.) and antibody (0.5 ml i.p.).

Hours 18: Penicillin: 1 ampoule is diluted in 3 ml solvent ad pen. ˜200 mg/ml; further dilution: 3 ml “200 mg/ml”+12.00 ml of saline ˜40 mg/ml.

The antibodies are diluted to 200 μg/ml:

### 3.00 ml of PdB26-5F12.1+12.00 ml of PBS

### 2.17 ml of OmpA6-4B6.1+12.83 ml of PBS

The mice are treated with penicillin (0.25 ml s.c.) and antibody (0.5 ml i.p.).

Hours 48: Penicillin: 1 ampoule is diluted in 3 ml solvent ad pen. ˜200 mg/ml; further dilution: 3 ml “200 mg/ml”+12.00 ml of saline ˜40 mg/ml.

The mice are treated with penicillin (0.25 ml s.c.).

| | Cage | | | | 48 |
|---|---|---|---|---|---|
| | no. | No. of mice | 0 hours | 18 hours | hours |
| Grp 1 | 1 | 5 | Bacteria + 5F12.1 | 5F12.1 + PEN | PEN |
| | 2 | 5 | | | |
| | 3 | 4 | | | |
| Grp 2 | 4 | 5 | Bacteria + 5F12.1 | 5F12.1 | |
| | 5 | 5 | | | |
| | 6 | 4 | | | |
| Grp 3 | 7 | 5 | Bacteria + 6-4B6 | 6-4B6 + PEN | PEN |
| | 8 | 5 | | | |
| | 9 | 4 | | | |
| Grp 4 | 10 | 5 | Bacteria + 6-4B6 | 6-4B6 | |
| | 11 | 5 | | | |
| | 12 | 4 | | | |
| Grp 5 | 13 | 5 | Bacteria | PEN | PEN |
| | 14 | 5 | | | |
| | 15 | 3 | | | |
| Grp 6 | 16 | 5 | Bacteria | | |
| | 17 | 5 | | | |
| | 18 | 3 | | | |

Morning and afternoon the following days for the duration of the experiment: The mice are scored according to scale 1-4.

| inoculate | undiluted | 10⁻¹ | 10⁻² | 10⁻³ | 10⁻⁴ | 10⁻⁵ | CFU/ml |
|---|---|---|---|---|---|---|---|
| Pn. D39 | ∞ | ∞ | i.t. | 12/20 | 4/2 | | 8.0 × 10⁵ |

### Results

The survival of the mice is evaluated at 24 hours.

The results of the experiments performed using 26-5F12.1 is summarised in FIG. 4, showing an increase survival rate at 24 hours.

### Example 2

### Detection of Anti-Haemolytic Properties in Antibodies from Culture Supernatant

### Description

Antibodies against Pneumolysin can inhibit the lytic effect of Pneumolysin. The lytic effect is abolished in the presence of serum, thereby rendering it necessary to bind the antibodies and remove the serum by washing before performing an anti-haemolytic assay.

### Devices:

### Incubator 37° C.

### Pipettes

### Centrifuge

ELISA reader, BIO-TEK EL 800

### Digital Camera, Canon Powershot S20

### Materials:

### Tips

Reagent tray
Plate cover
96-well microwell plate (Nunc 260836-flat bottom)
Reacti-Bind Protein G coated microwell strips, Pierce no. 15133

### Reagents:

Rec. PdB, diluted in PBS w.10 mM DTT to 4 μg/ml

### Dithiothreitol (DTT)

### PBS, pH 7.4

Dem. H₂O
Sheep erythrocytes 50% in Alsever's Fluid, SSI no. 29431

### Buffers:

### PBS pH 7.4

PBS pH 7.4 with 0.05% Tween20

### Controls:

### Catching:

Negative: PBS pH 7.4 with 0.05% Tween20
Haemolysis: PBS pH 7.4 with 0.05% Tween20
Positive High PdB22-6E6 diluted to 10 μg/ml in PBS
Positive Low: PdB22-6E6 diluted to 2 μg/ml in PBS

### Samples:

Samples are undiluted culture supernatants with antibody concentrations expected to be 1-5 μg/ml.

### Procedure:

Strips are washed three times in PBS/0.05% Tween
Add 50 μl/well of PBS/0.05% Tw20 followed by 50 μl/well of undiluted culture supernatant or 50 μl/well of controls.
Incubate 1 h at room temperature.
Wash x4 with PBS (without Tween20)
50 μl PBS is added to each well, A1-B1 are added 100 μl/well.
Recombinant PdB is diluted to 4 μg/ml in pre-heated PBS and activated with 10 mM
DTT (final concentration) for 15 min at 37° C.
Add 50 μl/well of activated PdB, except for A1-B1.

### Incubate for 30 min at 37° C.

Sheep erythrocytes are washed thrice in PBS and resuspended to 2% vol/vol in PBS.
Add 50 μl to each well and incubate for 30 min at 37° C.
Centrifuge plates 5 min at 1000×g.
A digital image of the plate is obtained.
Carefully transfer 100 μl of supernatant to flat-bottomed microwells and read OD at 405 nm.

STRIP NO.

| | 1 | 2 | 3 | 4 |
|---|---|---|---|---|
| A | Negative | Sample 1 | Sample 5 | Sample 9 |
| B | Negative | Sample 1 | Sample 5 | Sample 9 |
| C | Haemolysis | Sample 2 | Sample 6 | Sample 10 |
| D | Haemolysis | Sample 2 | Sample 6 | Sample 10 |
| E | Positive High | Sample 3 | Sample 7 | Sample 11 |
| F | Positive High | Sample 3 | Sample 7 | Sample 11 |
| G | Positive Low | Sample 4 | Sample 8 | Sample 12 |
| H | Positive Low | Sample 4 | Sample 8 | Sample 12 |

### Example 3

### Determination of Ability of Antibody to Inhibit the Haemolytic Activity of Pneumolysin

### Description

Purified antibodies against Pneumolysin can inhibit the lytic effect seen on erythrocytes, representing a functional assay for the screening of antibodies.

### Devices:

### Incubator 37° C.

### Pipettes

### Centrifuge

ELISA reader, BIO-TEK EL 800

### Digital Camera, Canon Powershot S20

### Materials:

### Tips

Reagent tray
Plate cover
96-well microwell plate (Nunc 260170-U-shaped)
96-well microwell plate (Nunc 260836-flat bottom)

### Reagents:

Rec. Pneumolysin (PLY) or Rec. Pneumolysoid (PdB)
PdB Lot #P01103 0.2 mg/ml in PBS diluted to 10 μg/ml

### Dithiothreitol (DTT)

### PBS, pH 7.4

Dem. H₂O
Sheep erythrocytes 50% in Alsever's Fluid, SSI no.29431

### Buffer:

### PBS pH 7.4

PBS with 10 mM DTT

### Samples:

Purified antibody samples are diluted in PBS.

### Procedure:

Determining Haemolytic endpoint:

This is determined for each new batch of PLY or PdB. All samples are done in triplicates. Controls are:

### Blank: 100 μl Buffer (0% Haemolysis)

Total: 100 μl Dem. H₂O (100% Haemolysis)

A dilution series of PLY/PdB is prepared in PBS w. 10 mM DTT: 40-20-10-5-2.5-1.25-0.625-0.3125 μg/ml. Add 100 μl to each well and incubate 15 min at 37° C. Sheep erythrocytes (50%) are washed three times in PBS and restored to 2% vol/vol. Add 50 pt to each well and incubate for 30 min at 37° C. Centrifuge 5 min at 1000×g.

A digital image of the plate is obtained.

100 μl supernatant is transferred to a flat bottom microwell plate and read at 405 nm. Twice the concentration of Pneumolysin giving 90% haemolysis is used as standard concentration in the inhibition assay.

### Inhibition Assay:

All tests are done in duplicates round-bottom microwell plates.

Controls are:

### Blank=100 μl PBS

Total Haemolysis=100 μl dem. H₂O

### Negative=50 μl Pneumolysin+50 μl PBS

Pneumolysin: Pool PdB 031201, 0.5 mg/ml diluted to 20 μg/ml=1 μg/well PLY/PdB is diluted in pre-heated PBS and activated with 10 mM DTT (final concentration) for 15 min at 37° C.
50 μl antibody dilution is added to each well followed by 50 μl activated PLY/PdB.

The plate is incubated for 30 min at 37° C.

Sheep blood is washed thrice in PBS and restored to 2% vol/vol.

Add 50 μl to each well and incubate plate for 30 min at 37° C.

Centrifuge 5 min at 1000×g.

A digital image of the plate is obtained.

100 μl of supernatant is transferred to a second flat bottom microwell plate (plate 2) and read at 405 nm (an example is shown in table 1). The titer is determined as the dilution of antibody which inhibits 50% haemolysis and is included in table 4 below.

### Samples:

All purified antibodies are diluted to 500 μg/ml in PBS.
S1=Ra-a-Pneumolysin
S2=OmpA17-10C7 031024
S3=22-6E6.5 040224
S4=26-5F12.1 040520
S5=26-23C2.2 040319
S6=26-18G8.2 040319
S7=26-30H10.2 040319
S8=28-10E7.2 040514
S9=26-14G4 040305
s10=13-2E12.1 031105
S11=22-1C11.1 031211

### Plate Setup:

### Plate 1:

| | | 2 | 3 | 4 | 5 | 6 | 7 | 8 | 9 | 10 | 11 | 12 |
|---|---|---|---|---|---|---|---|---|---|---|---|---|
| | 1 | S1 | S2 | S3 | S4 | S5 | S6 | S7 | S8 | S9 | S10 | S11 |
| A | Blank | 500 μg/ml | | | | | | | | | | |
| B | Blank | 100 μg/ml | | | | | | | | | | |
| C | Total | 20 μg/ml | | | | | | | | | | |
| D | Total | 4 μg/ml | | | | | | | | | | |
| E | Negative | 800 ng/ml | | | | | | | | | | |
| F | Negative | 160 ng/ml | | | | | | | | | | |
| G | | 32 ng/ml | | | | | | | | | | |
| H | | 6 ng/ml | | | | | | | | | | |

### Plate 2:

| | | | | | | | | | | | |
|---|---|---|---|---|---|---|---|---|---|---|---|
| | 2 | 3 | 4 | 5 | 6 | 7 | 8 | 9 | 10 | 11 | 12 |
| 1 | S1 | S2 | S3 | S4 | S5 | S6 | S7 | S8 | S9 | S10 | S11 |

| | | |
|---|---|---|
| A | Blank | 500 μg/ml |
| B | Blank | 100 μg/ml |
| C | Total | 20 μg/ml |
| D | Total | 4 μg/ml |
| E | Negative | 800 ng/ml |
| F | Negative | 160 ng/ml |
| G | | 32 ng/ml |
| H | | 6 ng/ml |

The data relating to sample 1 to 11 are shown in the tables here below.

TABLE 1
OD at 405 nm.
Blank 0.05
Total 1.15
Negative 1.18

| | Sample no. | | | | | | | | | | |
|---|---|---|---|---|---|---|---|---|---|---|---|
| | 1 | 2 | | | | | | | | | |
| Antibody, | Ra- | OmpA17- | 3 | 4 | 5 | 6 | 7 | 8 | 9 | 10 | 11 |
| ng/ml | a-Pneumoly- | 10C7 | 22-6E6.5 | 26-5F12.1 | 26-23C2.2 | 26-18G8.2 | 26-30H10.2 | 28-10E7.2 | 26-14G4 | 13-2E12.1 | 22-1C11.1 |
| 500000 | 1.15 | 1.23 | 0.05 | 0.05 | 0.05 | 0.05 | 0.05 | 0.05 | 0.06 | 1.17 | 1.23 |
| 100000 | 1.15 | 1.16 | 0.08 | 0.05 | 0.05 | 0.05 | 0.04 | 0.05 | 0.05 | 1.11 | 1.18 |
| 20000 | 1.14 | 1.17 | 0.06 | 0.05 | 0.04 | 0.04 | 0.05 | 0.07 | 0.05 | 1.13 | 1.16 |
| 4000 | 1.14 | 1.16 | 0.04 | 0.06 | 0.04 | 0.14 | 0.04 | 0.68 | 0.12 | 1.12 | 1.15 |
| 800 | 1.14 | 1.17 | 0.09 | 0.09 | 0.06 | 0.92 | 0.11 | 1.03 | 0.70 | 1.13 | 1.15 |
| 160 | 1.11 | 1.17 | 0.13 | 0.09 | 0.06 | 1.09 | 1.06 | 1.11 | 1.15 | 1.12 | 1.14 |
| 32 | 1.11 | 1.13 | 1.09 | 1.13 | 1.08 | 1.15 | 1.12 | 1.14 | 1.14 | 1.13 | 1.16 |
| 6 | 1.14 | 1.16 | 1.15 | 1.15 | 1.13 | 1.14 | 1.12 | 1.17 | 1.19 | 1.14 | 1.16 |

The % of haemolysis is calculated from the obtained data (table 1) and shown in table 2 here below.

TABEL 2
Haemolysis in %.

| | Ra-a- | | | | | | | | | | |
|---|---|---|---|---|---|---|---|---|---|---|---|
| | PdB | Dummy | 22-6E6.5 | 26-5F12.1 | 26-23C2.2 | 26-18G8.2 | 26-30H10.2 | 28-10E7.2 | 26-14G4 | 13-2E12.1 | 22-1C11.1 |
| 6 | 96 | 98 | 97 | 97 | 96 | 96 | 95 | 99 | 100 | 96 | 98 |
| 32 | 94 | 96 | 92 | 95 | 91 | 97 | 95 | 96 | 96 | 96 | 98 |
| 160 | 94 | 99 | 11 | 8 | 5 | 92 | 90 | 94 | 97 | 95 | 96 |
| 800 | 96 | 99 | 7 | 8 | 5 | 78 | 9 | 87 | 59 | 95 | 98 |
| 4000 | 97 | 98 | 4 | 5 | 3 | 12 | 4 | 58 | 10 | 95 | 97 |
| 20000 | 96 | 99 | 5 | 4 | 4 | 3 | 5 | 6 | 4 | 95 | 98 |
| 100000 | 98 | 98 | 7 | 4 | 4 | 4 | 4 | 4 | 5 | 94 | 99 |
| 500000 | 97 | 104 | 4 | 5 | 4 | 4 | 5 | 4 | 5 | 99 | 104 |

The % of inhibition is calculated from the obtained data (table 2) and shown in table 3 here below.

TABEL 3
% of inhibition of haemolysis.

| | Ra-a- | | | | | | | | | | |
|---|---|---|---|---|---|---|---|---|---|---|---|
| | PdB | Dummy | 22-6E6.5 | 26-5F12.1 | 26-23C2.2 | 26-18G8.2 | 26-30H10.2 | 28-10E7.2 | 26-14G4 | 13-2E12.1 | 22-1C11.1 |
| 6 | 3.9 | 1.7 | 2.8 | 3.0 | 4.1 | 3.7 | 5.2 | 1.3 | −0.4 | 3.7 | 1.9 |
| 32 | 5.8 | 4.2 | 7.6 | 4.8 | 8.9 | 2.7 | 5.1 | 4.0 | 3.6 | 4.4 | 2.1 |
| 160 | 5.9 | 1.3 | 89.1 | 92.5 | 95.1 | 7.7 | 10.1 | 5.8 | 3.0 | 5.4 | 3.6 |
| 800 | 3.6 | 1.3 | 92.5 | 92.1 | 94.7 | 21.9 | 90.9 | 13.0 | 40.9 | 4.6 | 2.5 |
| 4000 | 3.3 | 2.0 | 96.3 | 95.1 | 96.8 | 88.0 | 96.4 | 42.3 | 90.0 | 5.2 | 2.6 |
| 20000 | 4.1 | 0.7 | 94.7 | 96.1 | 96.4 | 96.6 | 95.5 | 94.3 | 95.9 | 4.6 | 1.9 |
| 100000 | 2.5 | 1.7 | 92.9 | 96.1 | 96.1 | 96.2 | 96.2 | 95.7 | 95.4 | 6.3 | 0.7 |
| 500000 | 3.0 | −3.7 | 95.5 | 95.4 | 95.5 | 95.7 | 95.4 | 95.5 | 95.2 | 0.9 | −4.0 |

Graphic illustrations of the results are depicted in FIG. 5.

The titer of the antibodies was determined based on the data described above and summarized in table 4 here below.

TABEL 4
The titter of selected antibodies.

| | Anti haemolytic actitity |
|---|---|
| Mab 0.5 μg/ml | ED50, ng/ml |
| 17-10C7.1 | >500 |
| 22-6E6.5 | <0.100 |
| 26-5F12.1 | <0.100 |
| 26-23C2.2 | <0.100 |
| 13-2E12.1 | >500 |
| 22-1C11.1 | >500 |
| 27-11A8 | >500 |
| 28-10E7.2 | >500 |

### Example 4

### Affinity Characterization of Anti-Pneumolysin HuMabs

Avidity measurements were made by flowing one concentration of mAbs on antigen coated surface.

### Methods & Materials:

Material coated on chin: Protein-G Chip type: CM5. Chip prepared on: Sep. 16, 2003
Coating density: FC1 & 3=blank, FC2=6286 RUs, FC4=6700RUs
Coating conditions: Conc. of protein=5 μg/mL, dilution buffer=sodium acetate, pH=4.5

### Running Buffer: HBS-EP.

### Reagents:

Antibodies (purified):

| | | |
|---|---|---|
| 1. | 4E8 | 0.94 mg/mL |
| 2. | 22-6E6 | 2.50 mg/mL |
| 3. | 26-23C2 | 3.40 mg/mL |
| 4. | 26-5F12 | 1.26 mg/mL |
| 5. | 22-1C11 | 5.80 mg/mL |
| 6. | 13-2E12 | 1.03 mg/mL |
| 7. | 10-3G7.2 | 1.10 mg/mL |
| 8. | 10-5G3.3 | 0.82 mg/mL |
| 9. | 10-14A5.2 | 0.91 mg/mL |
| 10. | 10-5G3.2 | 1.14 mg/mL |

Antigen: 0.6 mg/mL, 57 kDa (full length w/His-tag)

### Experimental Conditions:

Capture (Ab) Conc: 20 ug/mL concentration, 200 uL @ 50 uL/min flow rate
Association time: 4 min.
Dissociation time: 20 min.
Regeneration of chip: one pulse of 17 uL of 50 mM NaOH+75 NaCl @ 75 uL/min flow rate

### Results:

The estimate affinity and rate constants from this experiment are listed in Table 1. here below. The first few seconds of association and dissociation have been fit to a 1:1 Langmuir model to obtain the affinity and rate constants.

TABLE 1
Affinity and rate constants of Pneumolysin antibodies.

| | | Kₒₙ × 105 | K_{off} × 10−4 |
|---|---|---|---|
| Sample ID | K_{D} × 10−9 (M) | (1/Ms) | (1/s) |
| 4E8 | 0.22 | 11.6 | 2.5 |
| 22-6E6 | 0.31 | 13 | 4.0 |
| 26-23C2 | 0.69 | 5.2 | 3.6 |
| 26-5F12 | 0.82 | 5.3 | 4.3 |
| 22-1C11 | 11.7 | 0.62 | 0.7 |
| 13-2E12 | 24.7 | 1.24 | 3.1 |
| 10-3G7.2 | 0.66 | 0.95 | 30.6 |
| 10-5G3.3 | 1.1 | 0.39 | 0.44 |
| 10-14A5.2 | 28.7 | 0.71 | 20.3 |
| 10-5G3.2 | 0.66 | 0.41 | 0.27 |

### Example 5

### Generation of Anti-CD64×Anti-Pneumolysin 5-9A7 Bispecific Antibody

F(ab′)₂ fragments of each of the HuMAbs, anti-CD64 (88.53), and anti-Pneumolysin are generated by pepsin digestion and purified to homogeneity by Superdex 200 gel filtration chromatography. Size exclusion HPLC is performed, and by this type of analysis both of the F(ab′)₂ fragments are >95% pure.

A Fab′ fragment of the 88.53 is generated by mild reduction of the inter-heavy chain disulfide bonds of the F(ab′)₂ fragment with mercaptoethanolamine (MEA). The exact reducing conditions are determined prior to conjugation in small-scale experiments. Size exclusion HPLC is performed, and by this type of analysis the 88.53 Fab′ is >90% pure.

The Fab′ fragment of the 88.53 is separated from free MEA by G-25 column chromatography. The Fab′ fragment is incubated with dinitrothiobenzoate (DTNB) to generate a Fab-TNB conjugate.

A Fab′ fragment of the anti-Pneumolysin antibody is generated by mild reduction of the inter-heavy chain disulfide bonds of the F(ab′)₂ fragment with mercaptoethanolamine (MEA). The exact reducing conditions are determined prior to conjugation in small-scale experiments. Size exclusion HPLC is performed, and by this type of analysis the Fab′ is >90% pure.

The Fab′ fragment is separated from free MEA by G-25 column chromatography and mixed with 88.53 Fab-TNB at a 1:1 molar ratio overnight at room temperature.

The bispecifc antibody is purified from contaminating Fab′ molecules by Superdex 200 size exclusion chromatography, and the purified molecule is analyzed by HPLC.

For control anti-CD64×anti-CD89 Bispecific Antibody are generated. F(ab′)₂ fragments of each of the HuMAbs, anti-CD64 (88.53), and anti-CD89 (14A8) are generated by pepsin digestion and purified to homogeneity by Superdex 200 gel filtration chromatography. Size exclusion HPLC is performed, and by this type of analysis both of the F(ab′)₂ fragments are >95% pure.

A Fab′ fragment of the 88.53 is generated by mild reduction of the inter-heavy chain disulfide bonds of the F(ab′)₂ fragment with mercaptoethanolamine (MEA). The exact reducing conditions are determined prior to conjugation in small-scale experiments. Size exclusion HPLC is performed, and by this type of analysis the 88.53 Fab′ is >90% pure.

The Fab′ fragment of the 88.53 is separated from free MEA by G-25 column chromatography. The Fab′ fragment is incubated with dinitrothiobenzoate (DTNB) 16a and 16b to generate a Fab-TNB conjugate.

A Fab′ fragment of the 14A8 is generated by mild reduction of the inter-heavy chain disulfide bonds of the F(ab′)₂ fragment with mercaptoethanolamine (MEA). The exact reducing conditions are determined prior to conjugation in small-scale experiments. Size exclusion HPLC is performed, and by this type of analysis the 14A8 Fab′ is >95% pure.

The Fab′ fragment of the 14A8 is separated from free MEA by G-25 column chromatography and mixed with 88.53 Fab-TNB at a 1:1 molar ratio overnight at room temperature.

The bispecific antibody is purified from contaminating Fab′ molecules by Superdex 200 size exclusion chromatography, and the purified molecule is analyzed by HPLC. The 88.53×14A8 bispecific antibody is purified to near homogeneity.

Characterization of the Binding Specificity of the anti-CD64×Anti-Pneumolysin Bispecific Antibody—Bispecific ELISA
1. ELISA plates are coated with recombinant Pneumolysin, 50 μl/well, 5 μg/ml and incubated overnight at 4° C.
2. The plates are blocked with 5% BSA in PBS.
3. Titrations of the bispecific antibody are added to the plate. Controls include the anti-CD64×anti-CD89 bispecific (control bispecific) and the F(ab′)₂ fragments of the anti-CD64 Ab, 88.53 or of the anti-Pneumolysin Ab.
4. The plates are then incubated with a supernatant containing a fusion protein consisting of soluble CD64 linked to the Fc portion of human IgM.
5. The plates are finally incubated with an alkaline phosphatase labelled goat anti-human IgM antibody. Positive wells are detected with the alkaline phosphatase substrate.

### Characterization of the Binding Specificity of the Anti-CD64×Anti-Pneumolysin Bispecific Antibody—Binding to CD64 on Human CD64-Transgenic Mice

Blood is taken from CD64 transgenic mice or from non-transgenic littermates, and incubated with the 88.53×anti-Pneumolysin bispecific antibody at a concentration of 30 μg/ml for 30 minutes at room temperature.

The blood is washed and then incubated with an FITC-labelled anti-human IgG anti-body for 30 minutes at room temperature. The red blood cells are lysed and the remaining leukocytes are analyzed for staining by flow cytometry. Regions corresponding to the lymphocyte, monocyte, and neutrophil populations are gated and analyzed separately.

Human CD64 is expressed on monocytes and, to a lesser extent, neutrophils of CD64 transgenic mice. As in humans, CD64 is not expressed by lymphocytes of the transgenic mice. The bispecific antibody binds to CD64 transgenic monocytes and neutrophils, but not to any cell populations derived from non-transgenic mice.

### Example 6

### Sequencing of Monoclonal Antibody

The DNA encoding antibodies according to the present invention are sequenced as described below for the antibody 26-5F12.1.

Total RNA was isolated from hybridoma cells using STAT60 reagent (BioGenesis) and converted into cDNA for use as a template in PCR. Agarose gel analysis showed a high yield of the extracted RNA from the pellet (FIG. (8A)

cDNA was created from the RNA. Heavy chain and light chain variable regions were amplified using Heavy Primers and Light Primer Mix from Amersham Biosciences. The PCR products were analysed on a Tris-acetate-EDTA agarose gel. The PCR using these primers on the cDNA gave the bands shown in FIG. 8B.

Direct cloning of the PCR products gave poor transformation efficiency, so the PCR products were gel purified and cloned. Samples positive in the PCRs were cloned into the pCR4-TOPO vector in the TOPO TA Cloning Kit (Invitrogen).

The purified VL and VH PCR products were cloned into a sequencing vector and positive transformants were determined by colony PCR (FIG. 8C).

All positive clones were picked (normally 3) for each chain and sequenced with both forward and reverse sequencing primers. The clones were sequenced by the dideoxy method with the BigDye V3.1 DNA sequencing kit (Applied Biosystems).

Sequencing analysis identified five correct clones for the variable heavy chain and seven for the variable light chain of monoclonal antibody 26-5F 12.1. The DNA and protein sequences for each of these clones are shown below.

### Monoclonal Antibody 26-5F 12.1 Sequencing Results

26-5F 12.1 VH Clone 4 DNA Sequence
AGGTGCAGCTGCAGGAGTCAGGGGCTGAGGTGAAGAAGCCTGGGGCCTCA
GTGAAGGTTTCCTGCACGGCTTCTGGATACATCTTCACTAGCTATGCTAT
ACATTGGGTGCGCCAGGCCCCCGGACAAAGGCTTGAGTGGATGGGATGGA
TCAACGCTGGCTATGGTAACACAAAATATTCACAGAAGTTCCAGGGCAGA
GTCAGCATTACCAGGGACAAATCCGCGAGCACAGCCTACATGGAGTTGAG
CAGCCTGAGATCTGAAGACACGGCTGTGTATTACTGTGCGAGAAGGGGGC
AGCAGCTGGCCTTTGACTACTGGGGCCAAGGGACCACGGTCACCGTCTCC
TCA
26-5F 12.1 VH Clone 4 Protein Sequence
VQLQESGAEVKKPGASVKVSCTASGYIFTSYAIHWVRQAPGQRLEWMetG
WINAGYGNTKYSQKFQGRVSITRDKSASTAYMetELSSLRSEDTAVYYCA
RRGQQLAFDYWGQGTTVTVSS
26-5F 12.1 VH Clone 3 DNA Sequence
AGGTGAAGCTGCAGGAGTCTGGGGCTGAGGTGAAGAAGCCTGGGGCCTCA
GTGAAGGTTTCCTGCACGGCTTCTGGATACATCTTCACTAGCTATGCTAT
ACATTGGGTGCGCCAGGCCCCCGGACAAAGGCTTGAGTGGATGGGATGGA
TCAACGCTGGCTATGGTAACACAAAATATTCACAGAAGTTCCAGGGCAGA
GTCAGCATTACCAGGGACAAATCCGCGAGCACAGCCTACATGGAGTTGAG
CAGCCTGAGATCTGAAGACACGGCTGTGTATTACTGTGCGAGAAGGGGGC
AGCAGCTGGCCTTTGACTACTGGGGCCAAGGGACCACGGTCACCGTCTCC
TCA
26-5F 12.1 VH Clone 3 Protein Sequence
VKLQESGAEVKKPGASVKVSCTASGYIFTSYAIHWVRQAPGQRLEWMetG
WINAGYGNTKYSQKFQGRVSITRDKSASTAYMetELSSLRSEDTAVYYCA
RRGQQLAFDYWGQGTTVTVSS
26-5F 12.1 VH Clone 6 DNA Sequence
AGGTGAAGCTGCAGCAGTCTGGGGCTGAGGTGAAGAAGCCTGGGGCCTCA
GTGAAGGTTTCCTGCACGGCTTCTGGATACATCTTCACTAGCTATGCTAT
ACATTGGGTGCGCCAGGCCCCCGGACAAAGGCTTGAGTGGATGGGATGGA
TCAACGCTGGCTATGGTAACACAAAATATTCACAGAAGTTCCAGGGCAGA
GTCAGCATTACCAGGGACAAATCCGCGAGCACAGCCTACATGGAGTTGAG
CAGCCTGAGATCTGAAGACACGGCTGTGTATTACTGTGCGAGAAGGGGGC
AGCAGCTGGCCTTTGACTACTGGGGCCAAGGGACCACGGTCACCGTCTCC
TC
26-SF 12.1 VH Clone 6 Protein Sequence
VKLQQSGAEVKKPGASVKVSCTASGYIFTSYAIHWVRQAPGQRLEWMetG
WINAGYGNTKYSQKFQGRVSITRDKSASTAYMetELSSLRSEDTAVYYCA
RRGQQLAFDYWGQGTTVTVS
26-5F 12.1 VH Clone 15 DNA Sequence
AGGTGAAGCTGCAGCAGTCTGGGGCTGAGGTGAAGAAGCCTGGGGCCTCA
GTGAAGGTTTCCTGCACGGCTTCTGGATACATCTTCACTAGCTATGCTAT
ACATTGGGTGCGCCAGGCCCCCGGACAAAGGCTTGAGTGGATGGGATGGA
TCAACGCTGGCTATGGTAACACAAAATATTCACAGAAGTTCCAGGGCAGA
GTCAGCATTACCAGGGACAAATCCGCGAGCACAGCCTACATGGAGTTGAG
CAGCCTGAGATCTGAAGACACGGCTGTGTATTACTGTGCGAGAAGGGGGC
AGCAGCTGGCCTTTGACTACTGGGGCCAAGGGACCACGGTCACCGTCTCC
TC
26-5F 12.1 VH Clone 15 Protein Sequence
VKLQQSGAEVKKPGASVKVSCTASGYIFTSYAIHWVRQAPGQRLEWMetG
WINAGYGNTKYSQKFQGRVSITRDKSASTAYMetELSSLRSEDTAVYYCA
RRGQQLAFDYWGQGTTVTVS
26-SF 12.1 VH Clone 10 DNA Sequence
AGGTGAAGCTGCAGGAGTCAGGGGCTGAGGTGAAGAAGCCTGGGGCCTCA
GTGAAGGTTTCCTGCACGGCTTCTGGATACATCTTCACTAGCTATGCTAT
ACATTGGGTGCGCCAGGCCCCCGGACAAAGGCTTGAGTGGATGGGATGGA
TCAACGCTGGCTATGGTAACACAAAATATTCACAGAAGTTCCAGGGCAGA
GTCAGCATTACCAGGGACAAATCCGCGAGCACAGCCTACATGGAGTTGAG
CAGCCTGAGATCTGAAGACACGGCTGTGTATTACTGTGCGAGAAGGGGGC
AGCAGCTGGCCTTTGACTACTGGGGCCAAGGGACCACGGTCACCGTCTCC
TCA
26-5F 12.1 VH Clone 10 Protein Sequence
VKLQESGAEVKKPGASVKVSCTASGYIFTSYAIHWVRQAPGQRLEWMetG
WINAGYGNTKYSQKFQGRVSITRDKSASTAYMetELSSLRSEDTAVYYCA
RRGQQLAFDYWGQGTTVTVSS
26-5F 12.1 VL Clone 2 DNA Sequence
GACATCCAGATGACGCAGTCTCCAGGCACCCTGTCTTTGTCTCCAGGGGA
AAGAGCCACCCTCTCCTGCAGGGCCAGTCAGAGTGTTAGCAGCAGCTACT
TAGCCTGGTACCAGCAGAAACCTGGCCAGGCTCCCAGGCTCCTCATCTAT
GGTGCATCCAGCAGGGCCACTGGCATCCCAGACAGGTTCAGTGGCAGTGG
GTCTGGGACAGACTTCACTCTCACCATCAGCAGACTGGAGCCTGAAGATT
TTGCAGTGTATTACTGTCAGCAGTATGGTAGCTCACCATTCACTTTCGGC
CCTGGCACCAAGCTGGAAATCAAACGG
26-5F 12.1 VL Clone 2 Protein Sequence
DIQMetTQSPGTLSLSPGERATLSCRASQSVSSSYLAWYQQKPGQAPRLL
IYGASSRATGIPDRFSGSGSGTDFTLTISRLEPEDFAVYYCQQYGSSPFT
FGPGTKLEIKR
26-5F 12.1 VL Clone 3 DNA Sequence
GACATCCAGATGACGCAGTCTCCAGGCACCCTGTCTTTGTCTCCAGGGGA
AAGAGCCACCCTCTCCTGCAGGGCCAGTCAGAGTGTTAGCAGCAGCTACT
TAGCCTGGTACCAGCAGAAACCTGGCCAGGCTCCCAGGCTCCTCATCTAT
GGTGCATCCAGCAGGGCCACTGGCATCCCAGACAGGTTCAGTGGCAGTGG
GTCTGGGACAGACTTCACTCTCACCATCAGCAGACTGGAGCCTGAAGATT
TTGCAGTGTATTACTGTCAGCAGTATGGTAGCTCACCATTCACTTTCGGC
CCTGGCACCAAGCTGGAAATCAAACGG
26-5F 12.1 VL Clone 3 Protein Sequence
DIQMetTQSPGTLSLSPGERATLSCRASQSVSSSYLAWYQQKPGQAPRLL
IYGASSRATGIPDRFSGSGSGTDFTLTISRLEPEDFAVYYCQQYGSSPFT
FGPGTKLEIKR
26-5F 12.1 VL Clone 4 DNA Sequence
GACATCCAGATGACGCAGTCTCCAGGCACCCTGTCTTTGTCTCCAGGGGA
AAGAGCCACCCTCTCCTGCAGGGCCAGTCAGAGTGTTAGCAGCAGCTACT
TAGCCTGGTACCAGCAGAAACCTGGCCAGGCTCCCAGGCTCCTCATCTAT
GGTGCATCCAGCAGGGCCACTGGCATCCCAGACAGGTTCAGTGGCAGTGG
GTCTGGGACAGACTTCACTCTCACCATCAGCAGACTGGAGCCTGAAGATT
TTGCAGTGTATTACTGTCAGCAGTATGGTAGCTCACCATTCACTTTCGGC
CCTGGCACCAAGCTGGAAATCAAACGG
26-5F 12.1 VL Clone 4 Protein Sequence
DIQMetTQSPGTLSLSPGERATLSCRASQSVSSSYLAWYQQKPGQAPRLL
IYGASSRATGIPDRFSGSGSGTDFTLTISRLEPEDFAVYYCQQYGSSPFT
FGPGTKLEIKR
26-5F 12.1 VL Clone 5 DNA Sequence
GACATCCAGATGACTCAGTCTCCAGGCACCCTGTCTTTGTCTCCAGGGGA
AAGAGCCACCCTCTCCTGCAGGGCCAGTCAGAGTGTTAGCAGCAGCTACT
TAGCCTGGTACCAGCAGAAACCTGGCCAGGCTCCCAGGCTCCTCATCTAT
GGTGCATCCAGCAGGGCCACTGGCATCCCAGACAGGTTCAGTGGCAGTGG
GTCTGGGACAGACTTCACTCTCACCATCAGCAGACTGGAGCCTGAAGATT
TTGCAGTGTATTACTGTCAGCAGTATGGTAGCTCACCATTCACTTTCGGC
CCTGGCACCAAGCTGGAAATCAAACGG
26-SF 12.1 VL Clone 5 Protein Sequence
DIQMetTQSPGTLSLSPGERATLSCRASQSVSSSYLAWYQQKPGQAPRLL
IYGASSRATGIPDRFSGSGSGTDFTLTISRLEPEDFAVYYCQQYGSSPFT
FGPGTKLEIKR
26-5F 12.1 VL Clone 6 DNA Sequence
GACATCCAGATGACACAGTCTCCAGGCACCCTGTCTTTGTCTCCAGGGGA
AAGAGCCACCCTCTCCTGCAGGGCCAGTCAGAGTGTTAGCAGCAGCTACT
TAGCCTGGTACCAGCAGAAACCTGGCCAGGCTCCCAGGCTCCTCATCTAT
GGTGCATCCAGCAGGGCCACTGGCATCCCAGACAGGTTCAGTGGCAGTGG
GTCTGGGACAGACTTCACTCTCACCATCAGCAGACTGGAGCCTGAAGATT
TTGCAGTGTATTACTGTCAGCAGTATGGTAGCTCACCATTCACTTTCGGC
CCTGGCACCAAGCTGGAAATCAAACGG
26-SF 12.1 VL Clone 6 Protein Sequence
DIQMetTQSPGTLSLSPGERATLSCRASQSVSSSYLAWYQQKPGQAPRLL
IYGASSRATGIPDRFSGSGSGTDFTLTISRLEPEDFAVYYCQQYGSSPFT
FGPGTKLEIKR
26-SF 12.1 VL Clone 10 DNA Sequence
GACATCCAGATGACACAGTCTCCAGGCACCCTGTCTTTGTCTCCAGGGGA
AAGAGCCACCCTCTCCTGCAGGGCCAGTCAGAGTGTTAGCAGCAGCTACT
TAGCCTGGTACCAGCAGAAACCTGGCCAGGCTCCCAGGCTCCTCATCTAT
GGTGCATCCAGCAGGGCCACTGGCATCCCAGACAGGTTCAGTGGCAGTGG
GTCTGGGACAGACTTCACTCTCACCATCAGCAGACTGGAGCCTGAAGATT
TTGCAGTGTATTACTGTCAGCAGTATGGTAGCTCACCATTCACTTTCGGC
CCTGGCACCAAGCTGGAAATCAAACGG
26-5F 12.1 VL Clone 10 Protein Sequence
DIQMetTQSPGTLSLSPGERATLSCRASQSVSSSYLAWYQQKPGQAPRLL
IYGASSRATGIPDRFSGSGSGTDFTLTISRLEPEDFAVYYCQQYGSSPFT
FGPGTKLEIKR
26-5F 12.1 VL Clone 12 DNA Sequence
GACATCCAGATGACGCAGTCTCCAGGCACCCTGTCTTTGTCTCCAGGGGA
AAGAGCCACCCTCTCCTGCAGGGCCAGTCAGAGTGTTAGCAGCAGCTACT
TAGCCTGGTACCAGCAGAAACCTGGCCAGGCTCCCAGGCTCCTCATCTAT
GGTGCATCCAGCAGGGCCACTGGCATCCCAGACAGGTTCAGTGGCAGTGG
GTCTGGGACAGACTTCACTCTCACCATCAGCAGACTGGAGCCTGAAGATT
TTGCAGTGTATTACTGTCAGCAGTATGGTAGCTCACCATTCACTTTCGGC
CCTGGCACCAAGCTGGAAATCAAACGG
26-5F 12.1 VL Clone 12 Protein Sequence
DIQMetTQSPGTLSLSPGERATLSCRASQSVSSSYLAWYQQKPGQAPRLL
IYGASSRATGIPDRFSGSGSGTDFTLTISRLEPEDFAVYYCQQYGSSPFT
FGPGTKLEIKR

### Monoclonal Antibody 26-5F 12.1 Consensus Sequence

VH consensus protein sequence
VKLQESGAEVKKPGASVKVSCTASGYIFTSYAIHWVRQAPGQRLEWMetG
WINAGYGNTKYSQKFQGRVSITRDKSASTAYMetELSSLRSEDTAVYYCA
RRGQQLAFDYWGQGTTVTVSS
VL consensus Protein Sequence
DIQMetTQSPGTLSLSPGERATLSCRASQSVSSSYLAWYQQKPGQAPRLL
IYGASSRATGIPDRFSGSGSGTDFTLTISRLEPEDFAVYYCQQYGSSPFT
FGPGTKLEIKR

The sequences of the variable light and heavy chain of 26-5F 12.1 are shown in FIG. 3A, where the sequence of the CDRs is also included.

### Monoclonal Antibody 26-23 C2.2 Sequencing Analysis

RNA is extracted as described above showing a high yield (FIG. 9A).

cDNA was created from the RNA. The initial PCR reactions prepared to amplify the VL region were unsuccessful. New primers were ordered to amplify VHand VL in separate reactions. The PCR using these primers on the original cDNA gave the VH and VL bands shown in FIG. 9B.

The purified VH and VL PCR products were cloned into a sequencing vector and positive transformants were determined by colony PCR (FIG. 9C).

VH and VL clones were picked and sequenced. The sequence of 5 VH clones and 3 VL clones is shown here below.

### Monoclonal Antibody 26-23 C2.2 Sequencing Results

26-23 C2.2 VH Clone 1 DNA sequence
AGGTGAAGCTGCAGGAGTCAGGGGCTGAGGTGAAGAAGCCTGGGGCCTCA
GTGAAGGTTTCCTGCACGGCTTCTGGATACATCTTCACTAGCTATGCTAT
GCATTGGGTGCGCCAGGCCCCCGGACAAAGGCTTGAGTGGATGGGATGGA
TCAACGCTGGCTATGGTAACACAAAATATTCACAGAAGTTCCAGGGCAGA
GTCAGCATTACCAGGGACMATCCGCGAGCACAGCCTACATGGAGCTGACC
AGCCTGAGATCTGAGGACACGGCTGTGTATTACTGTGCGAGAAGGGGGCA
GCAGCTGGCCTTTGACTACTGGGGCCAAGGGACCACGGTCACCGTCTCCT
CA
26-23 C2.2 VH Clone 1 amino acid sequence
VKLQESGAEVKKPGASVKVSCTASGYIFTSYAMHWVRQAPGQRLEWMGWI
NAGYGNTKYSQKFQGRVSITRDKSASTAYMELTSLRSEDTAVYYCARRGQ
QLAFDYW-GQGTTVTVSS
26-23 C2.2 VH Clone 2 DNA sequence
AGGTGAAACTGCAGCTGTCTGGGGCTGAGGTGAAGAAGCCTGGGGCCTCA
GTGAAGGTTTCCTGCACGGCTTCTGGATACATCTTCACTAGCTATGCTAT
GCATTGGGTGCGCCAGGCCCCCGGACAAAGGCTTGAGTGGATGGGATGGA
TCAACGCTGGCTATGGTAACACAAAATATTCACAGAAGTTCCAGGGCAGA
GTCAGCATTACCAGGGACAAATCCGCGAGCACAGCCTACATGGAGCTGAC
CAGCCTGAGATCTGAGGACACGGCTGTGTATTACTGTGCGAGAAGGGGGC
AGCAGCTGGCCTTTGACTACTGGGGCCAAGGGACCACGGTCAACGTCTCC
TCA
26-23 C2.2 VH Clone 2 amino acid sequence
VKLQLSGAEVKKPGASVKVSCTASGYIFTSYAMHWVRQAPGQRLEWMGWI
NAGYGNTKYSQKFQGRVSITRDKSASTAYMELTSLRSEDTAVYYCARRGQ
QLAFDYW-GQGTTVNVSS
26-23 C2.2 VH Clone 3 DNA sequence
AGGTCAAACTGCAGGAGTCAGGGGCTGAGGTGAAGAAGCCTGGGGCCTCA
GTGAAGGTTTCCTGCACGGCTTCTGGATACATCTTCACTAGCTATGCTAT
GCATTGGGTGCGCCAGGCCCCCGGACAAAGGCTTGAGTGGATGGGATGGA
TCAACGCTGGCTATGGTAACACAAAATATTCACAGAAGTTCCAGGGCAGA
GTCAGCATTACCAGGGACAAATCCGCGAGCACAGCCTACATGGAGCTGAC
CAGCCTGAGATCTGAGGACACGGCTGTGTATTACTGTGCGAGAAGGGGGC
AGCAGCTGGCCTTTGACTACTGGGGCCAAGGGACCACGGTCACCGTCTCC
TCA
26-23 C2.2 Clone VH3 amino acid sequence
VKLQESGAEVKKPGASVKVSCTASGYIFTSYAMHWVRQAPGQRLEWMGWI
NAGYGNTKYSQKFQGRVSITRDKSASTAYMELTSLRSEDTAVYYCARRGQ
QLAFDYW-GQGTTVTVSS
26-23 C2.2 VH Clone 4 DNA sequence
AGCTCAAGCTGCAGGAGTCTGGGGCTGAGGTGAAGAAGCCTGGGGCCTCA
GTGAAGGTTTCCTGCACGGCTTCTGGATACATCTTCACTAGCTATGCTAT
GCATTGGGTGCGCCAGGCCCCCGGACAAAGGCTTGAGTGGATGGGATGGA
TCAACGCTGGCTATGGTAACACAAAATATTCACAGAAGTTCCAGGGCAGA
GTCAGCATTACCAGGGACAAATCCGCGAGCACAGCCTACATGGAGCTGAC
CAGCCTGAGATCTGAGGACACGGCTGTGTATTACTGTGCGAGAAGGGGGC
AGCAGCTGGCCTTTGACTACTGGGGCCAAGGGACCACGGTCACCGTCTCC
TCA
26-23 C2.2 VH Clone 4 amino acid sequence
LKLQESGAEVKKPGASVKVSCTASGYIFTSYAMHWVRQAPGQRLEWMGWI
NAGYGNTKYSQKFQGRVSITRDKSASTAYMELTSLRSEDTAVYYCARRGQ
QLAFDYW-GQGTTVTVSS
26-23 C2.2 VH Clone 5 DNA sequence
AGGTGCAGCTGCAGGAGTCAGGGGCTGAGGTGAAGAAGCCTGGGGCCTCA
GTGAAGGTTTCCTGCACGGCTTCTGGATACATCTTCACTAGCTATGCTAT
GCATTGGGTGCGCCAGGCCCCCGGACAAAGGCTTGAGTGGATGGGATGGA
TCAACGCTGGCTATGGTAACACAAAATATTCACAGAAGTTCCAGGGCAGA
GTCAGCATTACCAGGGACAAATCCGCGAGCACAGCCTACATGGAGCTGAC
CAGCCTGAGATCTGAGGACACGGCTGTGTATTACTGTGCGAGAAGGGGGC
AGCAGCTGGCCTTTGACTACTGGGGCCAAGGGACCACGGTCACCGTCTCC
TCA
26-23 C2.2 VH Clone 5 amino acid sequence
VQLQESGAEVKKPGASVKVSCTASGYIFTSYAMHWVRQAPGQRLEWMGWI
NAGYGNTKYSQKFQGRVSITRDKSASTAYMELTSLRSEDTAVYYCARRGQ
QLAFDYWGQGTTVTVSS
26-23C 2.2 VL clone 2 DNA sequnece
GACATCCGGGTGACCCAGTCTCCTGCTTCCTTAGCTGTATCTCTGGGGCA
GAGGGCCACCATCTCATACAGGGCCAGCAAAAGTGTCAGTACATCTGGCT
ATAGTTATACGCACTGGAACCAACAGAAACCAGGACAGCCACCCAGACTC
CTCATCTATCTTGTATCCAACCTAGAATCTGGGGTCCCTGCCAGGTTCAG
TGGCAGTGGGTCTGGGACAGACTTCACCCTCAACATCCATCCTGTGGAGG
AGGAGGATGCTGCAACCTATTACTGTCAGCACATTAGGGAGCTTACACGT
TCGGAGGGGGGACCAAGGTGGAAATCAAAA
26-23C 2.2 VL clone 2 Protein Sequence
DIRVTQSPASLAVSLGQRATISYRASKSVSTSGYSYTHWNQQKPGQPPRL
LIYLVSNLESGVPARFSGSGSGTDFTLNIHPVEEEDAATYYCQHIRELTR
SEGGPRWKSK
26-23C 2.2 VL clone 3 DNA Sequence
GACATCCAGATGACCCAGTCTCCTGCTTCCTTAGCTGTATCTCTGGGGCA
GAGGGCCACCATCTCATACAGGGCCAGCAAAAGTGTCAGTACATCTGGCT
ATAGTTATATGCACTGGAACCAACAGAAACCAGGACAGCCACCCAGACTC
CTCATCTATCTTGTATCCAACCTAGAATCTGGGGTCCCTGCCAGGTTCAG
TGGCAGTGGGTCTGGGACAGACTTCACCCTCAACATCCATCCTGTGGAGG
AGGAGGATGCTGCAACCTATTACTGTCAGCACATTAGGGAGCTTACACGT
TCGGAGGGGGGACCAAGCTGGAGATCAAAA
26-23C 2.2 VL clone 3 Protein Sequence
DIQMTQSPASLAVSLGQRATISYRASKSVSTSGYSYMHWNQQKPGQPPRL
LIYLVSNLESGVPARFSGSGSGTDFTLNIHPVEEEDAATYYCQHIRELTR
SEGGPSWRSK
26-23C 2.2 VL clone 4 DNA Sequence
GACATCCAGTTGACCCAGTCTCCTGCTTCCTTAGCTGTATCTCTGGGGCA
GAGGGCCACCATCTCATACAGGGCCAGCAAAAGTGTCAGTACATCTGGCT
ATAGTTATATGCACTGGAACCAACAGAAACCAGGACAGCCACCCAGACTC
CTCATCTATCTTGTATCCAACCTAGAATCTGGGGTCCCTGCCAGGTTCAG
TGGCAGTGGGTCTGGGACAGACTTCACCCTCAACATCCATCCTGTGGAGG
AGGAGGATGCTGCAACCTATTACTGTCAGCACATTAGGGAGCTTACACGT
TCGGAGGGGGGACCAAGGTGGAAATCAAAA
26-23C2.2 VL clone 4 Protein Sequence
DIQLTQSPASLAVSLGQRATISYRASKSVSTSGYSYMHWNQQKPGQPPRL
LIYLVSNLESGVPARFSGSGSGTDFTLNIHPVEEEDAATYYCQHIRELTR
SEGGPRWKSK

### Monoclonal Antibody 26-23C2.2 Consensus Sequences

VH consensus DNA sequence
AGGTGAAGCTGCAGGAGTCAGGGGCTGAGGTGAAGAAGCCTGGGGCCTCA
GTGAAGGTTTCCTGCACGGCTTCTGGATACATCTTCACTAGCTATGCTAT
GCATTGGGTGCGCCAGGCCCCCGGACAAAGGCTTGAGTGGATGGGATGGA
TCAACGCTGGCTATGGTAACACAAAATATTCACAGAAGTTCCAGGGCAGA
GTCAGCATTACCAGGGACAAATCCGCGAGCACAGCCTACATGGAGCTGAC
CAGCCTGAGATCTGAGGACACGGCTGTGTATTACTGTGCGAGAAGGGGGC
AGCAGCTGGCCTTTGACTACTGGGGCCAAGGGACCACGGTCACCGTCTCC
TCA
VH consensus amino acid sequence
VKLQESGAEVKKPGASVKVSCTASGYIFTSYAMHWVRQAPGQRLEWMGWI
NAGYGNTKYSQKFQGRVSITRDKSASTAYMELTSLRSEDTAVYYCARRGQ
QLAFDYW-GQGTTVTVSS
VL consensus DNA Sequence
GACATCCAGDTGACCCAGTCTCCTGCTTCCTTAGCTGTATCTCTGGGGCA
GAGGGCCACCATCTCATACAGGGCCAGCAAAAGTGTCAGTACATCTGGCT
ATAGTTATATGCACTGGAACCAACAGAAACCAGGACAGCCACCCAGACTC
CTCATCTATCTTGTATCCAACCTAGAATCTGGGGTCCCTGCCAGGTTCAG
TGGCAGTGGGTCTGGGACAGACTTCACCCTCAACATCCATCCTGTGGAGG
AGGAGGATGCTGCAACCTATTACTGTCAGCACATTAGGGAGCTTACACGT
TCGGAGGGGGGACCAAGGTGGAAATCAAAA
VL consensus Protein Sequence
DIQLTQSPASLAVSLGQRATISYRASKSVSTSGYSYMHWNQQKPGQPPRL
LIYLVSNLESGVPARFSGSGSGTDFTLNIHPVEEEDAATYYCQHIRELTR
SEGGPRWKSK

The sequences of the variable light and heavy chain of 26-23C2 are shown in FIG. 3B, where the sequence of the CDRs is also included.

### Monoclonal Antibody 22 1C 11 Sequencing Analysis

cDNA was created from mRNA. PCR reactions to amplify the VH and VL regions of the monoclonal antibody DNA gave the bands shown in FIG. 10A.

The purified VH and VL PCR products were cloned into a sequencing vector and positive transformants were determined by colony PCR (FIG. 10B):

Seven VH and six VL clones were picked for each chain and sequenced with both forward and reverse sequencing primers. Sequencing analysis identified 5 correct clones for the VH chain of monoclonal antibody 22-1C11.

The VL sequencing was of poorer quality. A further six clones were picked and sequenced to obtain a consensus sequence from a total of six clones.

The DNA and protein sequences for the positive VH and VL clones are shown below

Monoclonal Antibody 22-1C11 Sequencing results

22-1C 11 VH Clone 1 DNA sequence
AGGTGCAACTGCAGGAGTCTGGGGGAGGCGTGGTCCAGCCTGGGAGGTCC
CTAAGACTCTCCTGTGCAGCGTCTGGATTCACCTTCAGTAACTATGGCAT
GCACTGGGTCCGCCAGGCTCCAGGCAAGGGGCTGGAGTGGGTGGCAGTTA
TATGGTATGATGGAAGTAATAAATACTATGCAGACTTCGTGAAGGGCCGA
TTCACCATCTCCAGAGACAATTCCAAGAACACGCTGTATCTGCAAATGAA
CAGCCTGAGAGCCGAGGACACGGCTGTGTATTACTGTGCGAGAAGGGGAA
ATTACTATGGTTTGGGGAGCTTCTACTACTACGGTATGGACGTCTGGGGC
CAAGGGACCACGGTCACCG-TCTCCTCA
22-1C 11 VH Clone 1 Amino acid sequence:
VQLQESGGGWQPGRSLRLSCMSGFTFSNYGMHWVRQAPGKGLEWVAVIWY
DGSNKYYADFVKGRFTISRDNSKNTLYLQMNSLRAEDTAVYYCARRGNYY
GLGSFYYYGMDVWGQGTTVTVSS
22-1C 11 VH Clone 2 DNA sequence
AGGTGAAGCTGCAGGAGTCTGGGGGAGGCGTGGCCCAGCCTGGGAGGTCC
CTAAGACTCTCCTGTGCAGCGTCTGGATTCACCTTCAGTAACTATGGCAT
GCACTGGGTCCGCCAGGCTCCAGGCAAGGGGCTGGAGTGGGTGGCAGTTA
TATGGTATGATGGAAGTAATAAATACTATGCAGACTTCGTGAAGGGCCGA
TTCACCATCTCCAGAGACAATTCCAAGAACACGCTGTATCTGCAAATGAA
CAGCCTGAGAGCCGAGGACACGGCTGTGTATTACTGTGCGAGAAGGGGAA
ATTACTATGGTTTGGGGAGCTTCTACTACTACGGTATGGACGTCTGGGGC
CAAGGGACCACGGTCACCG-TCTCCTCA
22-1C 11 VH Clone 2 Amino Acid sequence:
VKLQESGGGVAQPGRSLRLSCAASGFTFSNYGMHWVRQAPGKGLEWVAVI
WYDGSNKYYADFVKGRFTISRDNSKNTLYLQMNSLRAEDTAVYYCARRGN
YYGLGSFYYYGMDVWGQGTTVTVSS
22-1C 11 VH Clone 3 DNA sequence
AGGTCCAACTGCAGGAGTCTGGGGGAGGCGTGGTCCAGCCTGGGAGGTCC
CTAAGACTCTCCTGTGCAGCGTCTGGATTCACCTTCAGTAACTATGGCAT
GCACTGGGTCCGCCAGGCTCCAGGCAAGGGGCTGGAGTGGGTGGCAGTTA
TATGGTATGATGGAAGTAATAAATACTATGCAGACTTCGTGAAGGGCCGA
TTCACCATCTCCAGAGACAATTCCAAGAACACGCTGTATCTGCAAATGAA
CAGCCTGAGAGCCGAGGACACGGCTGTGTATTACTGTGCGAGAAGGGGAA
ATTACTATGGTTTGGGGAGCTTCTACTACTACGGTATGGACGTCTGGGGC
CAAGGGACCACGGTCACCG-TCTCCTC
22-1C 11 VH Clone 3 Amino acid sequence
VQLQESGGGVVQPGRSLRLSCAASGFTFSNYGMHWVRQAPGKGLEWVAVI
WYDGSNKYYADFVKGRFTISRDNSKNTLYLQMNSLRAEDTAVYYCARRGN
YYGLGSFYYYGMDVWGQGTTVTVS
22-1C 11 VH Clone 4 DNA Sequence
AGGTCAAACTGCAGGAGTCTGGGGGAGGCGTGGTCCAGCCTGGGAGGTCC
CTAAGACTCTCCTGTGCAGCGTCTGGATTCACCTTCAGTAACTATGGCAT
GCACTGGGTCCGCCAGGCTCCAGGCAAGGGGCTGGAGTGGGTGGCAGTTA
TATGGTATGATGGAAGTAATAAATACTATGCAGACTTCGTGAAGGGCCGA
TTCACCATCTCCAGAGACAATTCCAAGAACACGCTGTATCTGCAAATGAA
CAGCCTGAGAGCCGAGGACACGGCTGTGTATTACTGTGCGAGAAGGGGAA
ATTACTATGGTTTGGGGAGCTTCTACTACTACGGTATGGACGTCTGGGGC
CAAGGGACCACGGTCACCG-TCTCCTCA
22-1C 11 VH Clone 4 Amino Acid Sequence
VKLQESGGGVVQPGRSLRLSCAASGFTFSNYGMHWVRQAPGKGLEWVAVI
WYDGSNKYYADFVKGRFTISRDNSKNTLYLQMNSLRAEDTAVYYCARRGN
YYGLGSFYYYGMDVWGQGTTVTVSS
22-1C 11 VH Clone 8 DNA sequence
AGGTGAAGCTGCAGGAGTCAGGGGGAGGCGTGGTCCAGCCTGGGAGGTCC
CTAAGACTCTCCTGTGCAGCGTCTGGATTCACCTTCAGTAACTATGGCAT
GCACTGGGTCCGCCAGGCTCCAGGCAAGGGGCTGGAGTGGGTGGCAGTTA
TATGGTATGATGGAAGTAATAAATACTATGCAGACTTCGTGAAGGGCCGA
TTCACCATCTCCAGAGACAATTCCAAGAACACGCTGTATCTGCAAATGAA
CAGCCTGAGAGCCGAGGACACGGCTGTGTATTACTGTGCGAGAAGGGGAA
ATTACTATGGTTTGGGGAGCTTCTACTACTACGGTATGGACGTCTGGGGC
CAAGGGACCACGGTCACCGTCTCCTCA
22-1C 11 VH Clone 8 Amino Acid Sequence
VKLQESGGGWQPGRSLRLSCAASGFTFSNYGMHWVRQAPGKGLEWVAVIW
YDGSNKYYADFVKGRFTISRDNSKNTLYLQMNSLRAEDTAVYYCARRGNY
YGLGSFYYYGMDVWGQGTTVTVSS
22-1C 11 VL Clone 3 DNA Sequence
GACATCCAGATGACCCAGTCTCCAGCCACCCTGTCTTTGTCTCCAGGGGA
AAGAGCCACCCTCTCCTGCAGGGCCAGTCAGAGTGTTAGCAGCTACTTAG
CCTGGTACCAGCAGAAACCTGGCCAGGCTCCCAGGCTCCTCATCTATGAT
GCATCCAACAGGGCCACTGGCATCCCAGCCAGGTTCAGTGGCAGTGGGCC
TGGGACAGACTTCACTCTCACCATCAGCAGCCTAGAGCCTGAAGATTTTG
CAGTTTATTACTGTCAGCAGCGTAGCAACTGGCATCCGACGTTCGGCCAA
GGCACCAAGCTGGAAATCAAACGG
22-1C 11 VL Clone 3 Amino Acid Sequence
DIQMTQSPATLSLSPGERATLSCRASQSVSSYLAWYQQKPGQAPRLLIYD
ASNRATGIPARFSGSGPGTDFTLTISSLEPEDFAVYYCQQRSNWHPTFGQ
GTKLXNQT
22-1C 11 VL Clone 6 DNA Sequence
ACACAGTNTCCNGCCNCCCTGTNTTNGTCTNCAGNGGAAAGANCCACCCT
NTCCNGCAGGNCCAGTCANAGTGTTNGCAGCTANTTAGCCTGGTACCAAC
AGAAANNTGGNCAGGCTCCCAGGCTCCTCATCTATGANGCATCCAACNGG
GCCACTGGCATCCCAGCCAGGTTCAGNGGCAGTGGGTNTGGGACAGACTT
CACTCTCACCATCAGCAGCNTAGAGCCTGAAGATTTNGCAGTTTATTACT
GTCAGCAGTGTAGCAACTGGCATCCGACATTCGGCCAAGGCACCAAGCTG
GAAATCAAANGN
Sequence of bad quality.
22-1C 11 VL Clone 7 DNA sequence
GACATCCAGATGACCCAGTTCCAGCCACCCTGTCTTTGTCTCCAGGGGAA
AGAGCCACCCTCTCCTGCAGGGCCAGTCAGAGTGTTAGCAGCTACTTAGC
CTGGTACCAACAGAAACCTGGCCAGGCTCCCAGGCTCCTCATCTATGATG
CATCCAACAGGGCCACTGGCATCCCAGCCAGGTTCAGTGGCAGTGGGTCT
GGGACAGACTTCACTCTCACCATCAGCAGCCTAGAGCCTGAAGATTTTGC
AGTTTATTACTGTCAGCAGTGTAGCAACTGGCATCCGACGTTCGGCCAAG
GCACCAAGCTGGAAAT-CAAACGG
22-1C 11 VL Clone 7 Amino Acid sequence
DIQMTQFQPPCLCLQGKEPPSPAGPVRVLAAT*PGTNRNLARLPGSSSMM
HPTGPLASQPGSVAVGLGQTSLSPSAA*SLKILQFITVSSVATGIRRSAK
APSWKSN
22-1C 11 VL Clone 11 DNA sequence
GACATCCAGATGACACAGTCTCCAGCCACCCTGTCTTTGTCTNCAGGGGA
AAGAGCCACCCTCTCCNGCAGGNCCAGTCAGAGTGTTAGCAGNTANTTAG
CCTGGTACCAACAGAAACCTGGCCAGGCTCCCAGGCTCCTCATCTATGAT
GCATCCAACAGGGCCACTGGCATCCCANNCAGGTTCAGTGGCAGTGGGTC
TGGGACAGACTTCACTCTCACCATCAGCAGCCTAGAGCCTGAAGATTTNG
CAGTTTATTACTGTCAGCAGTGTAGCAACTGGCATCNGACATTCGGCCAA
GGCACCAAGCTGGAAATCAAACGG
Sequence of bad quality.
22-1C 11 VL Clone 12 DNA sequence
GACATCCAGATGACACAGTCTCCAGCCACCCTGTCTTTGTCTCCAGGGGA
AAGAGCCACCCTCTCCTGCAGGGCCAGTCAGAGTGTTAGCAGCTACTTAG
CCTGGTACCAACAGAAACCTGGCCAGGCTCCCAGGCTCCTCATCTATGAT
GCATCCAACAGGGCCACTGGCATCCCAGCCAGGTTCAGTGGCAGTGGGTC
TGGGACAGACTTCACTCTCACCATCAGCAGCCTAGAGCCTGAAGATTTTG
CAGTTTATTACTGTCAGCAGTGTAGCAACTGGCATCCGACTTCGGCCAAG
GCACCAAGCTGGAAATCAAACGG
22-1C 11 VL Clone 12 Amino Acid sequence
DIQMTQSPATLSLSPGERATLSCRASQSVSSYLAWYQQKPGQAPRLLIYD
ASNRATGIPARFSGSGSGTDFTLTISSLEPEDFAVYYCQQCSNWHPTSAK
APSWKSN
22-1C 11 VL Clone 14 DNA sequence
GACATCCAGATGACACAGTCTCCAGCCACCCTGTCTTTGTCTCCAGGGGA
AAGAGCCACCCTCTCCTGCAGGGCCAGTCAGAGTGTTAGCAGCTACTTAG
CCTGGTACCAACAGAAACCTGGCCAGGCTCCCAGGCTCCTCATCTATGAT
GCATCCAACAGGGCCACTGGCATCCCAGCCAGGTTCAGTGGCAGTGGGTC
TGGGACAGACTTCACTCTCACCATCAGCAGCCTAGAGCCTGAAGATTTTG
CAGTTTATTACTGTCAGCAGTGTAGCAACTGGCATCNGACATTCGGCCAA
GGCACCAAGNTGGAAANCAAACGG
22-1C 11 VL Clone 14 Amino Acid sequence
DIQMTQSPATLSLSPGERATLSCRASQSVSSYLAWYQQKPGQAPRLLIYD
ASNRATGIPARFSGSGSGTDFTLTISSLEPEDFAVYYCQQCSNWHLTFGQ
GTK

### Monoclonal Antibody 22-1C11 Consensus Sequences

VH consensus DNA sequence
AGGTGAAACTGCAGGAGTCTGGGGGAGGCGTGGTCCAGCCTGGGAGGTCC
CTAAGACTCTCCTGTGCAGCGTCTGGATTCACCTTCAGTAACTATGGCAT
GCACTGGGTCCGCCAGGCTCCAGGCAAGGGGCTGGAGTGGGTGGCAGTTA
TATGGTATGATGGAAGTAATAAATACTATGCAGACTTCGTGAAGGGCCGA
TTCACCATCTCCAGAGACAATTCCAAGAACACGCTGTATCTGCAAATGAA
CAGCCTGAGAGCCGAGGACACGGCTGTGTATTACTGTGCGAGAAGGGGAA
ATTACTATGGTTTGGGGAGCTTCTACTACTACGGTATGGACGTCTGGGGC
CAAGGGACCACGGTCACCGTCTCCTCA
VH consensus Amino Acid sequence
VKLQESGGGVVQPGRSLRLSCAASGFTFSNYGMHWVRQAPGKGLEWVAVI
WYDGSNKYYADFVKGRFTISRDNSKNTLYLQMNSLRAEDTAVYYCARRGN
YYGLGSFYYYGMDVWGQGTTVTVSS
VL consensus DNA sequence
GACATCCAGATGACACAGTCTCCAGCCACCCTGTCTTTGTCTCCAGGGGA
AAGAGCCACCCTCTCCTGCAGGGCCAGTCAGAGTGTTAGCAGCTACTTAG
CCTGGTACCAACAGAAACCTGGCCAGGCTCCCAGGCTGCTCATCTATGAT
GCATCCAACAGGGCCACTGGCATCCCAGCCAGGTTCAGTGGCAGTGGGTC
TGGGACAGACTTCACTCTCACCATCAGCAGCCTAGAGCCTGAAGATTTTG
CAGTTTATTACTGTCAGCAGTGTAGCAACTGGCATCCGACATTCGGCCAA
GGCACCAAGCTGGAAATCAAACGG
VL consensus Amino Acid sequence
DIQMTQSPATLSLSPGERATLSCRASQSVSSYLAWYQQKPGQAPRLLIYD
ASNRATGIPARFSGSGSGTDFTLTISSLEPEDFAVYYCQQCSNWHPTFGQ
GTKLEIKR

The sequences of the variable light and heavy chain of 22-1C11 are shown in FIG. 3C, where the sequence of the CDRs is also included.

An alignment of the CDR sequences of 26-5F12, 26-23 C2 and 22 1C11 is shown in FIG. 11.

### Example 7

### Identification of Localisation of Epitopes

Synthetic peptide fragments of Pneumolysin of 12 amino acids, representing 28 amino acid residues of Pneumolysin are produced. The peptides overlap with neighbouring fragments with at least 8 amino acid residues. The peptides are shown in FIG. 6. Antibody binding to the fragments is tested in a standard ELISA assay as described here below. All peptides used are biotinylated peptides.

### Devices:

### Incubator at 37

### Pipettes

Elisa reader

### Material:

### Tips

Reagent tray
Plate cover
Reacti-Bind Streptavidin HBC Coated 96-well micro-well plates (Pierce)

### Reagents:

### Rabbit-α-Human IgG HRP (DAKO P0214)

OPD (o-Phenylenediamine)

### Buffers:

Wash and dilution buffer: PBS with 0.05% Tween20
Blocking buffer: wash buffer added 2% SMP (skimmed milk powder)

### Controls

Negative: blank
Negative: PsaA Peptide 9144 Biotin-KDPNNKEFYEKNLKEYTDKLDKLDK-NH2, 1 mg/ml 040630
Positive: PLY Peptide 10146 Biotin-ECTGLAWEWWRT-OH, 5 mg/ml

### Peptides:

Peptide “GNT-01” Biotin-RECTGLAWEWWR-OH, 5 mg/ml
Peptide “GNT-02” Biotin-IRECTGLAWEWW-OH, 5 mg/ml
Peptide “GNT-03” Biotin-KIRECTGLAWEW-OH, 50 μg/ml
Peptide “GNT-04” Biotin-VKIRECTGLAWE-OH, 50 μg/ml
Peptide “GNT-05” Biotin-SVKIRECTGLAW-OH, 50 μg/ml
Peptide “GNT-06” Biotin-LSVKIRECTGLA-OH, 50 μg/ml
Peptide “GNT-061” Biotin-NLSVKIRECTGL-OH, 50 μg/ml
Peptide “GNT-062” Biotin-RNLSVKIRECTG-OH, 50 μg/ml
Peptide “GNT-07” Biotin-CTGLAWEWWRTV-OH, 50 μg/ml
Peptide “GNT-08” Biotin-TGLAWEWWRTVY-OH, 50 μg/ml
Peptide “GNT-09” Biotin-GLAWEWWRTVYE-OH, 50 μg/ml
Peptide “GNT-10” Biotin-LAWEWWRTVYEK-OH, 50 μg/ml
Peptide “GNT-13” Biotin-EWWRTVYEKTDL-OH, 50 μg/ml
Peptide “GNT-14” Biotin-WWRTVYEKTDLP-OH, 50 μg/ml

### Procedure

The coated plates is rinsed well with 3×300 μl of wash buffer per well.

All peptides are diluted in PBS to 2.5 μg/ml. 100 μl is added per well and the plated is incubated for 1 hour at room temperature. The set up is shown here below.

The plate is flowingly rinsed with 3×200 μl of wash buffer per well and blocked for 30 min at RT with wash buffer including 2% SMP. Subsequently each well is rinsed with 3×200 μl of wash buffer. All Mabs are diluted to 0.5 μg/ml and 100 μl is added per well and the plate is incubated for 1 h at 37 C. The antibody is applied as shown below.

The plated is rinsed using 2×200 μl of wash buffer per well

The secondary antibody Rabbit-α-Human IgG HRP (DAKO P0214) is diluted 1:2000 in blocking buffer, 100 μl is added per well and the plate incubated 30 min at 37 C. Each well is rinsed with 3×200 μl of wash buffer and developed with OPD for 30 minutes.

Three independent experiments are performed and the result summarised here below. An overview of the results of plate 1 is shown in FIG. 7A and the results of plate 2 is shown in FIG. 7B.

Peptide set up (plate 1)

| | 1 | 2 | 3 | 4 | 5 | 6 | 7 | 8 | 9 |
|---|---|---|---|---|---|---|---|---|---|
| A | Blank | P9144 | P10146 | GNT-01 | GNT-02 | GNT-07 | GNT-08 | GNT-13 | GNT-14 |
| B | Blank | P9144 | P10146 | GNT-01 | GNT-02 | GNT-07 | GNT-08 | GNT-13 | GNT-14 |
| C | Blank | P9144 | P10146 | GNT-01 | GNT-02 | GNT-07 | GNT-08 | GNT-13 | GNT-14 |
| D | Blank | P9144 | P10146 | GNT-01 | GNT-02 | GNT-07 | GNT-08 | GNT-13 | GNT-14 |
| E | Blank | P9144 | P10146 | GNT-01 | GNT-02 | GNT-07 | GNT-08 | GNT-13 | GNT-14 |
| F | Blank | P9144 | P10146 | GNT-01 | GNT-02 | GNT-07 | GNT-08 | GNT-13 | GNT-14 |
| G | Blank | P9144 | P10146 | GNT-01 | GNT-02 | GNT-07 | GNT-08 | GNT-13 | GNT-14 |
| H | Blank | P9144 | P10146 | GNT-01 | GNT-02 | GNT-07 | GNT-08 | GNT-13 | GNT-14 |

Peptide set up (plate 2)

| | 1 | 2 | 3 | 4 | 5 | 6 | 7 | 8 | 9 | 10 | 11 | 12 | 13 | 14 | 15 | 16 | 17 |
|---|---|---|---|---|---|---|---|---|---|---|---|---|---|---|---|---|---|
| A | Blank | P9144 | P10146 | GNT-01 | GNT-02 | GNT-03 | GNT- | GNT- | GNT- | GNT- | GNT- | GNT- | GNT-08 | GNT-09 | GNT-10 | GNT-13 | GNT-14 |
| | | | | | | | 04 | 05 | 06 | 061 | 062 | 07 | | | | | |
| B | Blank | P9144 | P10146 | GNT- | GNT- | GNT- | GNT- | GNT- | GNT- | GNT- | GNT- | GNT- | GNT-08 | GNT-09 | GNT-10 | GNT-13 | GNT-14 |
| | | | | 01 | 02 | 03 | 04 | 05 | 06 | 061 | 062 | 07 | | | | | |
| C | Blank | P9144 | P10146 | GNT- | GNT- | GNT- | GNT- | GNT- | GNT- | GNT- | GNT- | GNT- | GNT-08 | GNT-09 | GNT-10 | GNT-13 | GNT-14 |
| | | | | 01 | 02 | 03 | 04 | 05 | 06 | 061 | 062 | 07 | | | | | |
| D | Blank | P9144 | P10146 | GNT- | GNT- | GNT- | GNT- | GNT- | GNT- | GNT- | GNT- | GNT- | GNT-08 | GNT-09 | GNT-10 | GNT-13 | GNT-14 |
| | | | | 01 | 02 | 03 | 04 | 05 | 06 | 061 | 062 | 07 | | | | | |
| E | Blank | P9144 | P10146 | GNT- | GNT- | GNT- | GNT- | GNT- | GNT- | GNT- | GNT- | GNT- | GNT-08 | GNT-09 | GNT-10 | GNT-13 | GNT-14 |
| | | | | 01 | 02 | 03 | 04 | 05 | 06 | 061 | 062 | 07 | | | | | |
| F | Blank | P9144 | P10146 | GNT- | GNT- | GNT- | GNT- | GNT- | GNT- | GNT- | GNT- | GNT- | GNT-08 | GNT-09 | GNT-10 | GNT-13 | GNT-14 |
| | | | | 01 | 02 | 03 | 04 | 05 | 06 | 061 | 062 | 07 | | | | | |
| G | Blank | P9144 | P10146 | GNT- | GNT- | GNT- | GNT- | GNT- | GNT- | GNT- | GNT- | GNT- | GNT-08 | GNT-09 | GNT-10 | GNT-13 | GNT-14 |
| | | | | 01 | 02 | 03 | 04 | 05 | 06 | 061 | 062 | 07 | | | | | |
| H | Blank | P9144 | P10146 | GNT- | GNT- | GNT- | GNT- | GNT- | GNT- | GNT- | GNT- | GNT- | GNT-08 | GNT-09 | GNT-10 | GNT-13 | GNT-14 |
| | | | | 01 | 02 | 03 | 04 | 05 | 06 | 061 | 062 | 07 | | | | | |

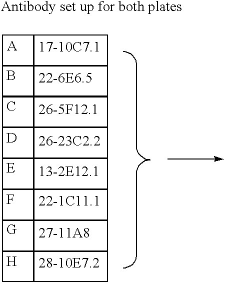

Elisa readings (plate 1):

| | | PsaA | PLY | | | | | | GNT- |
|---|---|---|---|---|---|---|---|---|---|
| Mab | Blank | pept. | Pept. | GNT-01 | GNT-02 | GNT-07 | GNT-08 | GNT-13 | 14 |
| 17-10C7.1 | 0.11 | 0.08 | 0.21 | 0.24 | 0.35 | 0.15 | 0.28 | 0.08 | 0.09 |
| 17-10C7.1 | 0.08 | 0.08 | 0.30 | 0.37 | 0.42 | 0.19 | 0.35 | 0.08 | 0.09 |
| 17-10C7.1 | 0.10 | 0.06 | 0.22 | 0.27 | 0.32 | 0.12 | 0.24 | 0.07 | 0.07 |
| 22-6E6.5 | 0.10 | 0.09 | 2.17 | 1.56 | 3.00 | 0.17 | 0.63 | 0.09 | 0.10 |
| 22-6E6.5 | 0.09 | 0.08 | 3.00 | 2.25 | 3.00 | 0.36 | 0.64 | 0.09 | 0.08 |
| 22-6E6.5 | 0.08 | 0.07 | 3.00 | 2.83 | 3.00 | 0.53 | 0.80 | 0.08 | 0.07 |
| 26-5F12.1 | 0.09 | 0.09 | 2.56 | 1.68 | 3.00 | 0.21 | 0.89 | 0.09 | 0.09 |
| 26-5F12.1 | 0.08 | 0.08 | 3.00 | 2.98 | 3.00 | 0.47 | 1.09 | 0.09 | 0.09 |
| 26-5F12.1 | 0.08 | 0.07 | 3.00 | 3.00 | 3.00 | 0.60 | 1.23 | 0.09 | 0.08 |
| 26-23C2.2 | 0.08 | 0.08 | 0.30 | 0.29 | 2.80 | 0.14 | 0.29 | 0.07 | 0.08 |
| 26-23C2.2 | 0.08 | 0.08 | 0.52 | 0.49 | 3.00 | 0.23 | 0.38 | 0.08 | 0.08 |
| 26-23C2.2 | 0.09 | 0.07 | 0.55 | 0.50 | 3.00 | 0.18 | 0.26 | 0.07 | 0.07 |
| 13-2E12.1 | 0.08 | 0.09 | 0.20 | 0.23 | 0.35 | 0.13 | 0.27 | 0.08 | 0.08 |
| 13-2E12.1 | 0.08 | 0.08 | 0.29 | 0.37 | 0.41 | 0.18 | 0.36 | 0.08 | 0.08 |
| 13-2E12.1 | 0.07 | 0.06 | 0.20 | 0.26 | 0.33 | 0.14 | 0.22 | 0.07 | 0.07 |
| 22-1C11.1 | 0.19 | 0.16 | 1.09 | 0.72 | 2.05 | 0.24 | 0.71 | 0.11 | 0.10 |
| 22-1C11.1 | 0.11 | 0.10 | 1.71 | 1.16 | 2.51 | 0.58 | 0.91 | 0.12 | 0.10 |
| 22-1C11.1 | 0.22 | 0.19 | 2.24 | 1.70 | 3.00 | 1.01 | 1.52 | 0.25 | 0.22 |
| 27-11A8 | 0.09 | 0.08 | 1.99 | 0.92 | 3.00 | 0.15 | 0.34 | 0.08 | 0.09 |
| 27-11A8 | 0.08 | 0.08 | 3.00 | 1.53 | 3.00 | 0.29 | 0.41 | 0.09 | 0.08 |
| 27-11A8 | 0.08 | 0.07 | 3.00 | 1.84 | 3.00 | 0.28 | 0.32 | 0.07 | 0.07 |
| 28-10E7.2 | 0.08 | 0.08 | 0.23 | 0.27 | 0.39 | 0.14 | 0.30 | 0.08 | 0.09 |
| 28-10E7.2 | 0.08 | 0.08 | 0.35 | 0.42 | 0.47 | 0.21 | 0.37 | 0.09 | 0.08 |
| 28-10E7.2 | 0.07 | 0.06 | 0.26 | 0.32 | 0.38 | 0.16 | 0.26 | 0.07 | 0.08 |

Overview of results from Elias readings of plate 1:

| | | Sequence | | | | | | | |
|---|---|---|---|---|---|---|---|---|---|
| | | KDPNNKEFYEKNL- | ECTGLA- | RECTGLA- | IRECTGLA- | CTGLAWE- | TGLAWEW- | EWWRTVY- | WWRTV- |
| | | KEYTDKLDKLDK | WEWWRT | WEWWR | WEWW | WWRTV | WRTVY | EKTDL | YEKTDLP |
| Mab | | PsaA | PLY | GNT- | GNT- | GNT- | GNT- | GNT- | GNT- |
| 0.5 μg/ml | Blank | pept. | Pept. | 01 | 02 | 07 | 08 | 13 | 14 |
| 17-10C7.1 | 0.11 | 0.08 | 0.21 | 0.24 | 0.35 | 0.15 | 0.28 | 0.08 | 0.09 |
| 22-6E6.5 | 0.10 | 0.09 | 2.17 | 1.56 | >3 | 0.17 | 0.63 | 0.09 | 0.10 |
| 26-5F12.1 | 0.09 | 0.09 | 2.56 | 1.68 | >3 | 0.21 | 0.89 | 0.09 | 0.09 |
| 26-23C2.2 | 0.08 | 0.08 | 0.30 | 0.29 | 2.80 | 0.14 | 0.29 | 0.07 | 0.08 |
| 13-2E12.1 | 0.08 | 0.09 | 0.20 | 0.23 | 0.35 | 0.13 | 0.27 | 0.08 | 0.08 |
| 22-1C11.1 | 0.19 | 0.16 | 1.09 | 0.72 | 2.05 | 0.24 | 0.71 | 0.11 | 0.10 |
| 27-11A8 | 0.09 | 0.08 | 1.99 | 0.92 | >3 | 0.15 | 0.34 | 0.08 | 0.09 |
| 28-10E7.2 | 0.08 | 0.08 | 0.23 | 0.27 | 0.39 | 0.14 | 0.30 | 0.08 | 0.09 |

Elisa readings plate 2:

| | | PsaA | PLY | GNT- | GNT- | GNT- | GNT- | GNT- | GNT- | GNT- | GNT- | GNT- | GNT- | GNT- | GNT- | GNT- | GNT- |
|---|---|---|---|---|---|---|---|---|---|---|---|---|---|---|---|---|---|
| Mab | Blank | pept. | Pept. | 01 | 02 | 03 | 04 | 05 | 06 | 061 | 062 | 07 | 08 | 09 | 10 | 13 | 14 |
| 17-10C7.1 | 0.062 | 0.077 | 0.119 | 0.117 | 0.185 | 0.081 | 0.077 | 0.08 | 0.072 | 0.077 | 0.088 | 0.071 | 0.083 | 0.065 | 0.07 | 0.062 | 0.068 |
| 22-6E6.5 | 0.072 | 0.074 | 2.25 | 2.175 | ****** | 2.111 | 0.235 | 0.133 | 0.096 | 0.151 | 0.109 | 0.108 | 0.323 | 0.322 | 0.09 | 0.089 | 0.088 |
| 26-5F12.1 | 0.071 | 0.069 | OUT | OUT | ****** | 2.814 | 0.262 | 0.154 | 0.104 | 0.152 | 0.107 | 0.132 | 0.761 | 0.293 | 0.09 | 0.09 | 0.092 |
| 26-23C2.2 | 0.068 | 0.061 | 1.112 | 0.764 | ****** | 0.711 | 0.133 | 0.108 | 0.089 | 0.112 | 0.098 | 0.094 | 0.16 | 0.105 | 0.076 | 0.076 | 0.086 |
| 13-2E12.1 | 0.067 | 0.065 | 0.128 | 0.118 | 0.171 | 0.078 | 0.074 | 0.075 | 0.069 | 0.072 | 0.077 | 0.08 | 0.085 | 0.067 | 0.066 | 0.066 | 0.074 |
| 22-1C11.1 | 0.567 | 0.539 | 1.577 | 1.274 | OUT | 1.103 | 0.748 | 0.816 | 0.56 | 0.61 | 0.844 | 0.681 | 0.73 | 0.475 | 0.403 | 0.264 | 0.257 |
| 27-11A8 | 0.082 | 0.073 | OUT | 1.699 | ****** | 0.938 | 0.15 | 0.148 | 0.088 | 0.158 | 0.112 | 0.113 | 0.175 | 0.095 | 0.076 | 0.071 | 0.076 |
| 28-10E7.2 | 0.074 | 0.08 | 0.174 | 0.166 | 0.251 | 0.127 | 0.098 | 0.097 | 0.086 | 0.098 | 0.107 | 0.089 | 0.106 | 0.081 | 0.071 | 0.072 | 0.072 |

Overview of results from Elisa readings of plate 2:

| | | Sequence | | | | | | |
|---|---|---|---|---|---|---|---|---|
| | | KDPNNKEFYEKNL- | ECTGLA- | RECTGLA- | IRECTGL- | KIRECT- | VKIRECT- | SVKIREC- |
| | | KEYTDKLDKLDK | WEWWRT | WEWWR | AWEWW | GLAWEW | GLAWE | TGLAW |
| | | PsaA | PLY | GNT- | GNT- | GNT- | GNT- | GNT- |
| Mab 0.5 μg/ml | Blank | pept. | Pept. | 01 | 02 | 03 | 04 | 05 |
| 17-10C7.1 | 0.06 | 0.08 | 0.12 | 0.12 | 0.19 | 0.08 | 0.08 | 0.08 |
| 22-6E6.5 | 0.07 | 0.07 | 2.25 | 2.18 | >3 | 2.11 | 0.24 | 0.13 |
| 26-5F12.1 | 0.07 | 0.07 | >3 | >3 | >3 | 2.81 | 0.26 | 0.15 |
| 26-23C2.2 | 0.07 | 0.06 | 1.11 | 0.76 | >3 | 0.71 | 0.13 | 0.11 |
| 13-2E12.1 | 0.07 | 0.07 | 0.13 | 0.12 | 0.17 | 0.08 | 0.07 | 0.08 |
| 22-1C11.1 | 0.57 | 0.54 | 1.58 | 1.27 | >3 | 1.10 | 0.75 | 0.82 |
| 27-11A8 | 0.08 | 0.07 | >3 | 1.70 | >3 | 0.94 | 0.15 | 0.15 |
| 28-10E7.2 | 0.07 | 0.07 | >3 | >3 | >3 | 2.81 | 0.26 | 0.15 |

| | Sequence | | | | | | | | |
|---|---|---|---|---|---|---|---|---|---|
| | LSVKIR- | NLSVKI- | RNLSVKI- | CTGLAWE- | TGLAWE- | GLAWEW- | LAWEWW- | EWWRTV- | WWRTVY- |
| Mab | ECTGLA | RECTGL | RECTG | WWRTV | WWRTVY | WRTVYE | RTVYEK | YEKTDL | EKTDLP |
| 0.5 μg/ml | GNT-06 | GNT-061 | GNT-062 | GNT-07 | GNT-08 | GNT-09 | GNT-10 | GNT-13 | GNT-14 |
| 17-10C7.1 | 0.07 | 0.08 | 0.09 | 0.07 | 0.08 | 0.07 | 0.07 | 0.06 | 0.07 |
| 22-6E6.5 | 0.10 | 0.15 | 0.11 | 0.11 | 0.32 | 0.32 | 0.09 | 0.09 | 0.09 |
| 26-5F12.1 | 0.10 | 0.15 | 0.11 | 0.13 | 0.76 | 0.29 | 0.09 | 0.09 | 0.09 |
| 26-23C2.2 | 0.09 | 0.11 | 0.10 | 0.09 | 0.16 | 0.11 | 0.08 | 0.08 | 0.09 |
| 13-2E12.1 | 0.07 | 0.07 | 0.08 | 0.08 | 0.09 | 0.07 | 0.07 | 0.07 | 0.07 |
| 22-1C11.1 | 0.56 | 0.61 | 0.84 | 0.68 | 0.73 | 0.48 | 0.40 | 0.26 | 0.26 |
| 27-11A8 | 0.09 | 0.16 | 0.11 | 0.11 | 0.18 | 0.10 | 0.08 | 0.07 | 0.08 |
| 28- | 0.10 | 0.15 | 0.11 | 0.13 | 0.11 | 0.08 | 0.07 | 0.07 | 0.07 |
| 10E7.2 | | | | | | | | | |

A graphic illustration of the results is shown in FIGS. 7A and 7B.

### REFERENCES

Alexander, J. E., R. A. Lock, C. A. M. Peeter, J. T. Poolman, P. W. Andrew, T. J. Mitchell, D. Hansman, and J. C. Paton. 1994. Immunization of mice with pneumolysin toxoid confers a significant degree of protection against at least nine serotypes of Streptococcus pneumoniae. Infect. Immun. 62:5683
AlonsoDeVelasco, E. Streptococcus pneumoniae: virulence factors, pathogenesis, and vaccines. Microbiol. Rev. 1995. December 59:591-603.
Anonymous. 1985. Acute respiratory infections in under fives: 15 million deaths a year. Lancet 2:699-701.
Barry, A. M., R. A. Lock, D. Hansman, and J. C. Paton. 1989. Contribution of autolysin to virulence of Streptococcus pneumoniae. Infect. Immun. 57:2324-2330.
Benton et al. 1997. Differences in virulence for mice among Streptococcus pneumoniae strains of capsular types 2, 3, 4, 5 and 6 are not attributable to differences in Pneumolysin production. Infect. Immun. 65:1237-1244.
Bonev, B. B., Gilbert, R. J. C., Andrew, P. W. et al. 2001. Structural analysis of the protein/lipid complexes associated with pore formation by the bacterial toxin pneumolysin. J. Biol. Chem. 276(8):5714-5719. Camara, M., G. J. Boulnois, P. W. Andrew, and T. J. Mitchell. 1994. A neuraminidase from Streptococcus pneumoniae has the features of a surface protein. Infect. Immun. 62:3688-3695.
Crain M J, et al. Pneumococcal surface protein A (PspA) is serologically highly variable and is expressed by all clinically important capsular serotypes of Streptococcus pneumoniae. Infect. Immun. 1990. October 58:3293-3299.
de los Toyos J R, et al. Functional analysis of pneumolysin by use of monoclonal antibodies. Infect. Immun. 1996. February 64:480-484.
Kuo, J., M. Douglas, H. K. Ree, and A. A. Lindberg. 1995. Characterization of a recombinant pneumolysin and its use as a protein carrier for pneumococcal type 18C conjugate vaccines. Infect. Immun. 63:2706-2713.
Lee C J, et al. Immunologic epitope, gene, and immunity involved in pneumococcal glycoconjugate. Crit. Rev. Microbiol. 1997. 23:121-142.
Lock, R. A., J. C. Paton, and D. Hansman. 1988. Comparative efficacy of pneumococcal neuraminidase and pneumolysin as immunogens protective against Streptococcus pneumoniae. Microb. Pathog. 5:461-467.
Lomholdt, H.1995. Evidence of recombination and an antigenically diverse immunoglobulin A1 protease among strains of Streptococcus pneumoniae. Infect. Immun. 63:4238-4243.
McDaniel L S, et al. Monoclonal antibodies against protease-sensitive pneumococcal anti-gens can protect mice from fatal infection with Streptococcus pneumoniae. J. Exp. Med. 1984. Aug.1. 160:386-397.
McDaniel L S, et al. PspA, a surface protein of Streptococcus pneumoniae, is capable of eliciting protection against pneumococci of more than one capsular type. Infect. Immun. 1991. Janurary 59:222-228.
Mitchell, T. J., F. Mendez, J. C. Paton, P. W. Andrew, and G. J. Boulnois. 1990. Comparison of pneumolysin genes and proteins from Streptococcus pneumoniae type 1 and 2. Nuclei. Acids. Res. 18:4010
Musher, D. M., Mediwala, R., Phan, H. M. et al. 2001. Nonspecificity of assaying for IgG antibody to pneumolysin in circulating immune complexes as a means to diagnose pneumococcal pneumonia. Clin. Infect. Dis. 32:534-538.
Paton, J. C., R. A. Lock, and D. Hansman. 1983. Effect of immunization with pneumolysin on survival time of mice challenged with Streptococcus pneumoniae. Infect. Immun. 40:548
Paton, J. C., R. A. Lock, C. J. Lee, J. P. Li, A. M. Barry, T. J. Mitchell, P. W. Andrew, D. Hansman, and G. J. Boulnois. 1991. Purification and immunogenicity of genetically obtained pneumolysin toxoids and their conjugation to Streptococcus pneumoniae type 19F polysaccharide. Infect. Immun. 59:2297-2304.
Rapola, S., Jannti, V., Haikala, R., Carlone, G. M., Sampson, J. S., Briles, D. E., Paton, J. C., Takala, A. K., Kilpi, T. M., and Käyhty,H.2000. Natural development of anti-bodies to pneumococcal surface protein A, pneumococcal surface adhesin A, and pneumolysin in relation to pneumococcal carriage and acute otitis media. J. Infect. Dis. 182:1146-1152.
Simell, B., Korkeila, M., Pursiainen, H., Kilpi, T. M., and Kaiyhty,H.2001. Pneumococcal carriage and otitis media induce salivary antibodies to pneumococcal surface adhesin A, pneumolysin, and pneumococcal surface protein A in children. J. Infect. Dis. 183:887-896.
Sorensen, U. B. Pneumococcal polysaccharide antigens: capsules and C-polysaccharide. An immunochemical study. Dan. Med. Bull. 1995. February 42:47-53.
Swiatlo, E., M. J. Crain, L. S. McDaniel, A. Brooks-Walter, T. J. Coffey, B. G. Spratt, D. A. Morrison, and D. E. Briles. 1996. DNA polymorphisms and variants penicillin-binding proteins as evidence that relatively penicillin-resistant pneumococci in Western Canada are clonally related. J. Infect. Dis. 174:884-888.
Tai, S. S., T. R. Wang, and C. J. Lee. 1997. Characterization of hemin binding activity of Streptococcus pneumoniae. Infect. Immun. 65:1083
Talkington D F, et al. A 43-kilodalton pneumococcal surface protein, PspA: isolation, protective abilities, and structural analysis of the amino-terminal sequence. Infect. Immun. 1991. April 59:1285-1289.

1. An isolated anti-haemolytic binding member comprising at least one binding domain capable of specifically binding Pneumolysin, wherein said binding domain recognizes an epitope in the N-terminal part of Pneumolysin corresponding to amino acid 1-436 of SEQ ID NO: 11.

2. The isolated binding member according to claim 1, wherein said binding domain recognizes an epitope in a region of Pneumolysin corresponding to amino acid 200-436 of SEQ ID NO: 11.

3. The isolated binding member according to claim 1, wherein the isolated binding member is a pure isolated binding member.

4. The isolated binding member according to claim 1, wherein the binding member is selected from an antibody or an immunologically active fragment thereof or a single chain thereof.

5. The isolated binding member according to claim 4, wherein the antibody is selected from: a monoclonal antibody, a polyclonal antibody, a mixture of monoclonal antibodies.

6. The isolated binding member according to claim 1, wherein the binding member is monospecific towards Pneumolysin.

7. The isolated binding member according to claim 1, wherein the binding member is bispecific having at least one portion specific towards Pneumolysin.

8. The isolated binding member according to claim 1, wherein the binding member is multispecific having at least one portion towards Pneumolysin.

9. The isolated binding member according to claim 1, wherein the binding domain is carried by a human antibody framework.

10. The isolated binding member according to claim 1, wherein the binding domain is carried by a humanised antibody framework.

11. The isolated binding member according to claim 1, wherein said binding domain recognizes an epitope comprised by SEQ ID NO: 27.

12. The isolated binding member according claim 1, wherein said binding domain recognizes an epitope in a sequence selected from: SEQ ID NO. 28, 29, 30 and 31.

13. The isolated binding member according to claim 1, wherein said binding domain recognizes an epitope in a sequence comprising amino acid 425-436 of Pneumolysin as identified by SEQ ID NO: 11.

14. The isolated binding member according to claim 1, wherein the binding domain comprises at least one amino acid sequence selected from SEQ ID NOs 3, 4, 5, 6, 7, 8, 9 and 10 or a homologue thereof.

15. The isolated binding member according to claim 1, wherein the binding domain comprises at least one amino acid sequence selected from SEQ ID NOs 12, 13, 14, 15, 16, 17, 18 and 10 or a homologue thereof.

16. The isolated binding member according to claim 1, wherein the binding domain comprises an amino acid sequence comprising the sequence identified by SEQ ID NO 10 or a homologue thereof.

17. The isolated binding member according to claim 1, wherein the binding domain comprises an amino acid sequence comprising the sequence selected from: SEQ ID NO 8, SEQ ID 17 or a homologue thereof.

18. The isolated binding member according to claim 1, wherein the binding domain comprises an amino acid sequence comprising the sequence selected from: SEQ ID 9, SEQ ID 18 or a homologue thereof.

19. The isolated binding member according to claim 1, wherein the binding member is capable of binding Pneumolysin from two or more different Pneumococcus serotypes.

20. The isolated binding member according to claim 14, wherein the homologue is at least 60% identical to at least one sequence selected from SEQ ID NOs 3, 4, 5, 6, 7, 8, 9, 10.

21. The isolated binding member according to claim 1, wherein the dissociation constant is less than 5×10⁻⁹ M.

22. The isolated binding member according to claim 1, wherein the binding domain is located in a V_{L} domain.

23. The isolated binding member according to claim 1, wherein the binding domain is located in a V_{H} domain.

24. The isolated binding member according to claim 1, wherein the binding domain is arranged as a complementarity-determining region (CDR) in the binding member.

25. The isolated binding member according to claim 4, wherein the antibody fragment is selected from Fab, Fab′, F(ab)₂ and Fv.

26. The binding member according to claim 1, comprising at least a first binding domain and a second binding domain, said first binding domain being capable of specifically binding Pneumolysin, and said second binding domain is different from said first binding domain.

27. The isolated binding member according to claim 26, wherein the second binding domain is capable of specifically binding a mammalian protein.

28. The isolated binding member according to claim 26, wherein the second binding domain is capable of specifically binding a mammalian cell selected from a leucocyte, a macrophage a lymphocyte, a neutrophilic cell a basophilic cell, an eosinophilic cell.

29. The isolated binding member according to claim 26, wherein the second binding domain is capable of specifically binding a Pneumococcus protein.

30. The isolated binding member according to claim 26, wherein second binding domain is capable of specifically binding a Pneumolysin epitope different from the first binding domain.

31. The isolated binding member according to claim 1, wherein the binding member comprises two binding domains.

32. The isolated binding member according to claim 31, wherein the two binding domains are linked through a spacer region.

33. An isolated nucleic acid molecule encoding at least a part of the binding member as defined in claim 1.

34. A vector comprising the nucleic acid molecule as defined in claim 33.

35. The vector according to claim 34, comprising a nucleotide sequence which regulates the expression of the binding member encoded by the nucleic acid molecule.

36. A host cell comprising the nucleic acid molecule as defined in claim 33.

37. A cell line engineered to express the binding member as defined in claim 1.

38. A method of detecting of diagnosing a disease or disorder associated with Pneumococcus in an individual comprising
providing a biological sample from said individual,
adding at least one binding member as defined in claim 1 to said biological sample,
detecting binding members bound to said biological sample, thereby detecting or diagnosing the disease or disorder.

39. A kit comprising at least one binding member as defined in claim 1, said binding member being labelled.

40. A pharmaceutical composition comprising at least one binding member as defined in claim 1.

41. The pharmaceutical composition according to claim 40, comprising at least two different binding members.

42. Use of a binding member as defined claim 1 for the production of a pharmaceutical composition.

43. Use of a binding member as defined claim 1 for the production of a pharmaceutical composition for the treatment of Pneumococcus infection.

44. A Pneumolysin peptide consisting of amino acid 1-436 of SEQ ID NO 11, fragments or variants thereof, recognized by the binding member as defined in claim 1.

45. A Pneumolysin peptide, fragment or variant thereof, comprising an amino acid sequence selected from SEQ ID NO 27, 28, 27, 30, 31, 32, 33, 34, 35e and 36.

46. A vaccine composition comprising a Pneumolysin peptide, wherein the Pneumolysin peptide, comprises an amino acid sequence or variant thereof selected from SEQ ID NO 27, 28, 29, 30, 31, 32, 33, 34, 35 and 36.

47. The vaccine according to claim 46, further comprising an adjuvant.

48. The vaccine according to claim 46, wherein the Pneumolysin peptide comprises amino acid 425-436 of SEQ ID NO 11, fragments or variants thereof, recognized by an isolated anti-haemolytic binding member comprising at least one binding domain capable of specifically binding Pneumolysin, wherein said binding domain recognizes an epitope in the N-terminal part of Pneumolysin corresponding to SEQ ID NO: 11.

49. The vaccine composition according to claim 46, wherein the Pneumolysin peptide, fragment or variant thereof is constituted by at the most 100

50. Use of a vaccine composition according to claim 46 for prophylactic treatment of Pneumococcus infection.

51. The isolated binding member according to claim 15, wherein the homologue is at least 60% identical to at least one sequence selected from SEQ ID NOs 10, 12, 13, 14, 15, 16, 17, 18.


SEQUENCE LISTING (26 sequences)

SEQ ID NO: 1 - protein, 12 aa, Homo sapiens
IRECTGLAWEWW

SEQ ID NO: 2 - protein, 16 aa, Homo sapiens
VKIRECTGLAWEWWRT

SEQ ID NO: 3 - protein, 109 aa, Artificial Sequence
  Variable light chain 26-5F12.1
DIQMTQSPGTLSLSPGERATLSCRASQSVSSSYLAWYQQKPGQAPRLLIYGASSRATGIPDRFSGSGSGTDFTLTISRLEPEDFAVYYCQQYGSSPFTFGPGTKLEIKR

SEQ ID NO: 4 - protein, 117 aa, Artificial Sequence
  Variable light chain 26-5F12.1
VKLQESGAEVKKPGASVKVSCTASGYIFTSYAIHWVRQAPGQRLEWMGWINAGYGNTKYSQKFQGRVSITRDKSASTAYMELSSLRSEDTAVYYCARRGQQLAFDYWGQGTTVTVSS

SEQ ID NO: 5 - protein, 12 aa, Artificial Sequence
  CDR1 light chain 26-F12.1
RASQSVSSSYLA

SEQ ID NO: 6 - protein, 7 aa, Artificial Sequence
  CDR2 light chain 26-5F12.1
GASSRAT

SEQ ID NO: 7 - protein, 6 aa, Artificial Sequence
  CDR3 llight chain 26-5F12.1
QQYGSS

SEQ ID NO: 8 - protein, 5 aa, Artificial Sequence
  CDR1 heavy chain 26-5F12.1
SYAIH

SEQ ID NO: 9 - protein, 14 aa, Artificial Sequence
  CDR2 heavy chain 26-5F12.1
WINAGYGNTKYSQK

SEQ ID NO: 10 - protein, 17 aa, Artificial Sequence
  CDR3 heavy chain 26-5F12.1
RGQQLAFDYWGQGTTVT

SEQ ID NO: 11 - protein, 471 aa, Homo sapiens
MANKAVNDFILAMNYDKKKLLTHQGESIENRFIKEGNQLPDEFVVIERKKRSLSTNTSDISVTATNDSRLYPGALLVVDETLLENNPTLLAVDRAPMTYSIDLPGLASSDSFLQVEDPSNSSVRGAVNDLLAKWHQDYGQVNNVPARMQYEKITAHSMEQLKVKFGSDFEKTGNSLDIDFNSVHSGEKQIQIVNFKQIYYTVSVDAVKNPGDVFQDTVTVEDLKQRGISAERPLVYISSVAYGRQVYLKLETTSKSDEVEAAFEALIKGVKVAPQTEWKQILDNTEVKAVILGGDPSSGARVVTGKVDMVEDLIQEGSRFTADHPGLPISYTTSFLRDNVVATFQNSTDYVETKVTAYRNGDLLLDHSGAYVAQYYITWDELSYDHQGKEVLTPKAWDRNGQDLTAHFTTSIPLKGNVRNLSVKIRECTGLAWEWWRTVYEKTDLPLVRKRTISIWGTTLYPQVEDKVEND

SEQ ID NO: 12 - protein, 110 aa, Artificial Sequence
  Variable light chain 26-23C2.2
DIQLTQSPASLAVSLGQRATISYRASKSVSTSGYSYMHWNQQKPGQPPRLLIYLVSNLESGVPARFSGSGSGTDFTLNIHPVEEEDAATYYCQHIRELTRSEGGPRWKSK

SEQ ID NO: 13 - protein, 117 aa, Artificial Sequence
  Variable heavy chain 26-23C2.2
VKLQESGAEVKKPGASVKVSCTASGYIFTSYAMHWVRQAPGQRLEWMGWINAGYGNTKYSQKFQGRVSITRDKSASTAYMELTSLRSEDTAVYYCARRGQQLAFDYWGQGTTVTVSS

SEQ ID NO: 14 - protein, 12 aa, Artificial Sequence
  CDR1 light chain 26-23C2.2
RASKSVSTSGYS

SEQ ID NO: 15 - protein, 7 aa, Artificial Sequence
  CDR2 light chain 26-23C2.2
LVSNLES

SEQ ID NO: 16 - protein, 6 aa, Artificial Sequence
  CDR3 light chain 26-23C2.2
QHIREL

SEQ ID NO: 17 - protein, 5 aa, Artificial Sequence
  CDR1 heavy chain 26-23C2.2
SYAMH

SEQ ID NO: 18 - protein, 14 aa, Artificial Sequence
  CDR2 heavy chain 26-23C2.2
WINAGYGNTKYSQK

SEQ ID NO: 19 - protein, 108 aa, Artificial Sequence
  Variable light chain 22-1C11
DIQMTQSPATLSLSPGERATLSCRASQSVSSYLAWYQQKPGQAPRLLIYDASNRATGIPARFSGSGSGTDFTLTISSLEPEDFAVYYCQQCSNWHPTFGQGTKLEIKR

SEQ ID NO: 20 - protein, 125 aa, Artificial Sequence
  Variable heavy chain 22-1C11
VKLQESGGGVVQPGRSLRLSCAASGFTFSNYGMHWVRQAPGKGLEWVAVIWYDGSNKYYADFVKGRFTISRDNSKNTLYLQMNSLRAEDTAVYYCARRGNYYGLGSFYYYGMDVWGQGTTVTVSS

SEQ ID NO: 21 - protein, 11 aa, Artificial Sequence
  CDR1 light  chain 22-1C11
RASQSVSSYLA

SEQ ID NO: 22 - protein, 7 aa, Artificial Sequence
  CDR2 light chain 22-1C11
DASNRAT

SEQ ID NO: 23 - protein, 6 aa, Artificial Sequence
  CDR3 light chain 22-1C11
QQCSNW

SEQ ID NO: 24 - protein, 6 aa, Artificial Sequence
  CDR1 heavy chain 22-1C11
SNYGMH

SEQ ID NO: 25 - protein, 14 aa, Artificial Sequence
  CDR2 heavy chain 22-1C11
VIWYDGSNKYYADF

SEQ ID NO: 26 - protein, 17 aa, Artificial Sequence
  CDR3 heavy chain 22-1C11
SFYYYGMDVWGQGTTVT